(19) United States
(12) Patent Application Publication
(10) Pub. No.: US 2021/0346336 A1
(43) Pub. Date: Nov. 11, 2021

(54) FURAN DERIVATIVES AS BROMODOMAIN INHIBITORS

(71) Applicant: GlaxoSmithKline Intellectual Property (No.2) Limited, Middlesex (GB)

(72) Inventors: Emmanuel Hubert DEMONT, Stevenage (GB); Jonathan Thomas SEAL, Stevenage (GB)

(21) Appl. No.: 17/271,272

(22) PCT Filed: Aug. 29, 2019
(86) PCT No.: PCT/EP2019/073081

§ 371 Date: Feb. 25, 2021

(30) Foreign Application Priority Data
Aug. 31, 2018 (GB) 1814167.1

(51) Int. Cl. A61K 31/341; C07D 307/68; C07D 307/93; C07D 405/06; C07D 471/04; A61K 31/343; A61K 31/443; A61K 31/437

(52) U.S. Cl. CPC A61K 31/341; C07D 307/68; C07D 307/93; A61K 31/437; C07D 471/04; A61K 31/343; A61K 31/443; C07D 405/06

(57) ABSTRACT

The present invention is directed to furan derivatives, pharmaceutical compositions comprising the compounds and the use of the compounds or the compositions in the treatment of various diseases

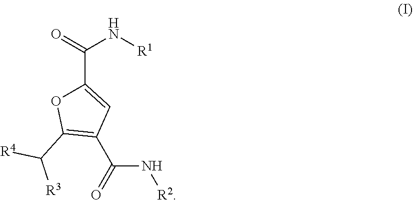

### FIELD OF THE INVENTION

The present invention is directed to furan derivatives which are bromodomain inhibitors, pharmaceutical compositions comprising the compounds and the use of the compounds or the compositions in the treatment of various diseases or conditions, for example acute or chronic autoimmune and/or inflammatory conditions, viral infections and cancer.

### BACKGROUND TO THE INVENTION

The genomes of eukaryotic organisms are highly organised within the nucleus of the cell. The long strands of duplex DNA are wrapped around an octomer of histone proteins (most usually comprising two copies of histones H2A, H2B, H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation and SUMOylation. These epigenetic marks are written and erased by specific enzymes, which place the tags on specific residues within the histone tail, thereby forming an epigenetic code, which is then interpreted by the cell to allow gene specific regulation of chromatin structure and thereby transcription.

Histone acetylation is most usually associated with the activation of gene transcription, as the modification loosens the interaction of the DNA and the histone octomer by changing the electrostatics. In addition to this physical change, specific proteins recognise and bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRDT) which contain tandem bromodomains capable of binding to two acetylated lysine residues in close proximity, increasing the specificity of the interaction. Numbering from the N-terminal end of each BET protein the tandem bromodomains are typically labelled Binding Domain 1 (BD1) and Binding Domain 2 (BD2) (Chung et al., J Med. Chem., 2011, 54, 3827-3838).

Chan et al. report that BET bromodomain inhibition suppresses transcriptional responses to cytokine-Jak-STAT signalling in a gene-specific manner in human monocytes, which suggests that BET inhibition reduces inflammation partially through suppression of cytokine activity. (Chan et al., Eur. J. Immunol., 2015, 45: 287-297).

Klein et al. report that the bromodomain protein inhibitor I-BET151 suppresses expression of inflammatory genes and matrix degrading enzymes in rheumatoid arthritis synovial fibroblasts, which suggests a therapeutic potential in the targeting of epigenetic reader proteins in rheumatoid arthritis. (Klein et al., Ann. Rheum. Dis., 2014, 0:1-8).

Park-Min et al. report that I-BET151 that targets bromo and extra-terminal (BET) proteins that ‘read’ chromatin states by binding to acetylated histones, strongly suppresses osteoclastogenesis. (Park-Min et al. Nature Communications, 2014, 5, 5418).

PCT patent applications WO2017/037116, WO2017/050714 and WO2017/060180 each describe a series of pyridone derivatives as bromodomain inhibitors. PCT patent applications WO2017/174621, WO2017/202742 and PCT/EP2018/054730 each describe a series of pyridine derivatives as bromodomain inhibitors. PCT patent application PCT/EP2018/054733 describes a series of pyrazole derivatives as bromodomain inhibitors.

### SUMMARY OF THE INVENTION

The invention is directed to compounds of formula (I)

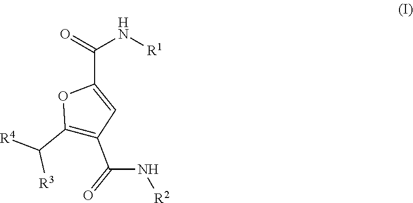

or a salt thereof
wherein:

R¹ is —C₁₋₃alkyl or cyclopropyl;

R² is —C₀₋₃alkyl-cycloalkyl, wherein the cycloalkyl group is optionally substituted with one, two or three R⁵ groups which may be the same or different; or

R² is —C₀₋₄alkyl-heterocyclyl or —(CH₂)ₚO-heterocyclyl wherein each heterocyclyl is optionally substituted by one or two R⁹ groups which may be the same or different; or R² is H, —CH₃, —C₂₋₆alkyl optionally substituted by one, two, three, four or five fluoro, —C₂₋₆alkylOR⁶, —C₂₋₆alkylNR^{10a}R^{11a}, —(CH₂)ₘSO₂C₁₋₃alkyl, —(CH₂)ₘSO₂NR¹⁰R¹¹, —(CH₂)ₘC(O)NR¹⁰R¹¹, —(CH₂)ₘCN, —(CH₂)ₘCO₂R⁶, —(CH₂)ₘNHCO₂C₁₋₄alkyl, —(CH₂)ₘNHC(O)C₁₋₄alkyl or —(CH₂)ₙheteroaryl wherein the heteroaryl is optionally substituted by one or two R⁷ groups which may be the same or different;

R³ is H, —C₁₋₄alkyl, cyclopropyl, fluoro, chloro, —CH₂F, —C₀₋₃alkylOR⁵ or —C₀₋₃alkylCN;

R⁴ is phenyl or a heteroaryl group wherein each are optionally substituted by one, two or three R⁷ groups which may be the same or different;

each R⁵ is independently halo, —C₀₋₆alkyl-R⁸, —O—C₂₋₆alkyl-R⁸, —OCH₂phenyl, —CN or —SO₂C₁₋₃alkyl;

R⁶ is H or —C₁₋₄alkyl;

each R⁷ is independently oxo, halo, —C₁₋₄alkyl optionally substituted by one, two or three fluoro, —C₀₋₃alkylOR⁶, —OC₂₋₃alkylOR⁶, —C₀₋₃alkylNR¹⁰R¹¹, —C₀₋₃alkyl-CONR¹⁰R¹¹, —CN, —SO₂—C₁₋₃alkyl, —SO₂NR¹⁰R¹¹ or —SO₂phenyl optionally substituted by —C₁₋₄alkyl;

R⁸ is H, —OR⁶, —NR¹⁰R¹¹ or heteroaryl;

each R⁹ is independently halo, —C₁₋₄alkyl, cyclopropyl, cyclobutyl, —CH₂CF₃, —CH₂CHF₂, —CH₂CH₂F, —OCH₂CH₂OR⁶, —C₀₋₃alkylOR⁶, —C₀₋₃alkylNR¹⁰R¹¹, —NHCH₂CH₂OR⁶, —NHCO₂C₁₋₄alkyl, oxo, —C(O)R⁶, —C(O)OR⁶ or —C(O)NR¹⁰R¹¹;

R¹⁰ and R¹¹ are each independently selected from H and —C₁₋₃alkyl; or R¹⁰ and R¹¹ may join together with the nitrogen to which they are attached, to form a 4 to 7-membered heterocyclyl optionally substituted by one or two substituents independently selected from —C₁₋₃alkyl optionally substituted with one, two or three fluorine atoms, —C₂₋₄alkylOH, —OH and F;

R^{10a} and R^{11a} are each independently selected from H and —C₁₋₃alkyl;

m is an integer selected from 2, 3 or 4;

n is an integer selected from 0, 1, 2, 3 or 4; and

p is an integer selected from 2, 3 or 4.

Compounds of the invention have been shown to be bromodomain inhibitors, in particular BD2 selective and may be useful in the treatment of various diseases or conditions, for example acute or chronic auto-immune and/or inflammatory conditions, for example rheumatoid arthritis and cancer. Accordingly, the invention is further directed to pharmaceutical compositions comprising a compound of formula (I), or a pharmaceutically acceptable salt thereof. The invention is still further directed to methods of treatment of diseases or conditions associated with bromodomains using a compound of formula (I) or a pharmaceutically acceptable salt thereof, or a pharmaceutical composition comprising a compound of formula (I) or a pharmaceutically acceptable salt thereof.

### DETAILED DESCRIPTION OF THE INVENTION

Compounds of formula (I) and salts thereof are referred to herein as “compounds of the invention”.

“BD2” refers to Binding Domain 2 of any of the the BET family of proteins BRD2, BRD3, BRD4 or BRDT.

“Alkyl” refers to a saturated hydrocarbon chain having the specified number of carbon atoms. For example, the term “C₁₋₃alkyl” and “C₁₋₄alkyl” as used herein refers to a straight or branched alkyl group having from 1 to 3 or 1 to 4 carbon atoms respectively. Further, the term “C₀₋₃alkyl” refers to a straight or branched alkyl group having from 0 (i.e. a bond) to 3 carbon atoms. Representative branched alkyl groups have one, two or three branches. An alkyl group may form part of a chain, for example, —C₀₋₃alkylOR⁶ refers to a straight or branched alkyl chain having from 0 (i.e. a bond) to 3 carbon atoms linked to a group R⁶. “Alkyl” includes, but is not limited to, methyl, ethyl, n-propyl, iso-propyl, n-butyl, iso-butyl, t-butyl, pentyl and hexyl.

“Cycloalkyl” refers to a saturated hydrocarbon mono or bicyclic ring or a saturated spiro-linked bicyclic hydrocarbon ring, having 3, 4, 5, 6, 7, 8, 9 or 10 member atoms in the ring. Suitable examples of cycloalkyl include, but are not limited to, cyclopropyl, cyclobutyl, cyclopentyl, cyclohexyl, cycloheptanyl, spiro[3.3]heptanyl, bicyclo[2.2.1]heptanyl, adamantyl, bicyclo[3.1.0]hexanyl and bicyclo[2.2.2]octanyl. “C₃₋₇cycloalkyl” refers to a saturated hydrocarbon mono or bicyclic ring or a saturated spiro-linked bicyclic hydrocarbon ring, having 3, 4, 5, 6 or 7 member atoms in the ring. Examples of C₃₋₇cycloalkyl groups include, but are not limited to, cyclopropyl, cyclobutyl, cyclopentyl, cyclohexyl, cycloheptanyl and bicyclo[3.1.0]hexanyl.

“Halo” refers to a halogen radical, for example, fluoro, chloro, bromo, or iodo.

“Heteroaryl” refers to a monocyclic or bicyclic group having 5, 6, 8, 9, 10 or 11 member atoms, including one, two or three heteroatoms independently selected from nitrogen, sulphur and oxygen, wherein at least a portion of the group is aromatic. The point of attachment to the rest of the molecule may be by any suitable carbon or nitrogen atom. Examples of “heteroaryl” groups include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, benzazepinyl, 2,3,4,5-tetrahydro-1H-benzo[d]azepinyl, indolizinyl, indolyl, indolinyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, pyrrolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

“C₅₋₆heteroaryl” refers to a monocyclic aromatic group having 5 or 6 member atoms, including 1, 2, 3 or 4 heteroatoms independently selected from nitrogen, sulphur and oxygen. The point of attachment to the rest of the molecule may be by any suitable carbon or nitrogen atom. Examples of “C₅₋₆heteroaryl” groups include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl and triazinyl.

“Heteroatom” refers to a nitrogen, sulfur, or oxygen atom.

“Heterocyclyl” refers to a non-aromatic heterocyclic monocyclic or bicyclic ring system containing 4, 5, 6, 7, 8, 9 or 10 ring member atoms, including one heteroatom and optionally containing a further heteroatom selected from nitrogen, oxygen or sulphur. Examples of “heterocyclyl” groups include, but are not limited to, azetidinyl, oxetanyl, thietanyl, pyrrolidinyl, pyrrolinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, homopiperazinyl, tetrahydropyranyl, dihydropyranyl, tetrahydrothiopyranyl, 1,3-dioxanyl, 1,4-dioxanyl, 1,3-oxathiolanyl, 1,3-oxathianyl, 1,3-dithianyl, 1,4-oxathiolanyl, 1,4-oxathianyl, 1,4-dithianyl, morpholinyl, thiomorpholinyl, hexahydro-1H-1,4-diazepinyl, azabicyclo[3.2.1]octyl, azabicyclo[3.3.1]nonyl, azabicylco[4.3.0]nonyl, oxabicyclo[2.2.1]heptyl, 1,1-dioxidotetrahydro-2H-thiopyranyl, 1,5,9-triazacyclododecyl, 3-oxabicyclo[3.1.0]hexanyl, 3-azabicyclo[3.1.0]hexanyl, (1r,5s)-3-oxabicyclo[3.1.0]hexanyl and (1r,5s)-3-azabicyclo[3.1.0]hexanyl.

“4 to 7-membered heterocyclyl” refers to a non-aromatic heterocyclic ring system containing 4, 5, 6 or 7 ring member atoms, including one heteroatom and optionally containing a further heteroatom selected from nitrogen, oxygen or sulphur. Examples of “4 to 7-membered heterocyclyl” groups include, but are not limited to, azetidinyl, pyrrolidinyl, piperidinyl, piperazinyl and morpholinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group attached to a chain or ring are not member atoms in the chain or ring.

“Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as rearrangement, cyclisation, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

“Pharmaceutically acceptable excipient” refers to a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of formula (I) or a pharmaceutically acceptable salt thereof when administered to a patient are avoided. In addition, each excipient must of course be pharmaceutically acceptable e.g. of sufficiently high purity.

“rac” refers to the racemic mixture of the compounds of formula (I).

The compounds of the invention may exist in solid or liquid form. In the solid state, the compounds of the invention may exist in crystalline or non-crystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates may involve non-aqueous solvents such as ethanol, iso-propyl alcohol, dimethylsulfoxide (DMSO), acetic acid, ethanolamine, and ethyl acetate, or they may involve water as the solvent that is incorporated into the crystalline lattice. Solvates wherein water is the solvent that is incorporated into the crystalline lattice are typically referred to as “hydrates”. Hydrates include stoichiometric hydrates as well as compositions containing variable amounts of water. The invention includes all such solvates.

It will be further appreciated that certain compounds of the invention that exist in crystalline form, including the various solvates thereof, may exhibit polymorphism (i.e. the capacity to occur in different crystalline structures). These different crystalline forms are typically known as “polymorphs”. The invention includes such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. It will be appreciated that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

The compounds according to formula (I) may contain one or more asymmetric centres (also referred to as a chiral centres) and may, therefore, exist as individual enantiomers, diastereoisomers, or other stereoisomeric forms, or as mixtures thereof. Chiral centres, such as chiral carbon atoms, may also be present in a substituent such as an alkyl group. Where the stereochemistry of a chiral centre present in formula (I), or in any chemical structure illustrated herein, is not specified, the structure is intended to encompass any stereoisomer and all mixtures thereof. Thus, compounds according to formula (I) containing one or more chiral centres may be used as racemic mixtures, enantiomerically-enriched mixtures, or as enantiomerically-pure individual stereoisomers. Accordingly, the present invention encompasses all isomers of the compounds of formula (I) whether as individual isomers isolated such as to be substantially free of the other isomer (i.e. pure) or as mixtures (i.e. racemic mixtures). An individual isomer isolated such as to be substantially free of the other isomer (i.e. pure) may be isolated such that less than 10%, particularly less than about 1%, for example less than about 0.1% of the other isomer is present.

Racemic compounds with a single stereocentre are denoted with either no stereochemistry (single bond) or have the annotation (+/−) or rac. Racemic compounds with two or more stereocentres where relative stereochemistry is known are denoted cis or trans as drawn in the structure. Resolved single enantiomers with unknown absolute stereochemistry but known relative stereochemistry are referred to with (R* or S*) with the appropriate relative stereochemistry depicted.

Where diastereoisomers are represented and only the relative stereochemistry is referred to, the bold or hashed solid bond symbols ( [symbol] ) are used. Where the absolute stereochemistry is known and the compound is a single enantiomer, the bold or hashed wedges symbols ( [symbol] ) are used as appropriate.

Individual stereoisomers of a compound according to formula (I) which contain one or more asymmetric centres may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. It will be appreciated that where the desired stereoisomer is converted into another chemical entity by one of the separation procedures described above, a further step is required to liberate the desired form. Alternatively, specific stereoisomers may be synthesised by asymmetric synthesis using optically active reagents, substrates, catalysts or solvents, or by converting one enantiomer to the other by asymmetric transformation.

It will be appreciated that, for compounds of formula (I) tautomers may be observed. Any comment relating to the biological activity of a tautomer should be taken to include both tautomers.

It is to be understood that the references herein to compounds of formula (I) and salts thereof covers the compounds of formula (I) as free bases, or as salts thereof, for example as pharmaceutically acceptable salts thereof. Thus, in one embodiment, the invention is directed to compounds of formula (I) as the free base. In another embodiment, the invention is directed to compounds of formula (I) and salts thereof. In a further embodiment, the invention is directed to compounds of formula (I) and pharmaceutically acceptable salts thereof.

Because of their potential use in medicine, salts of the compounds of formula (I) are desirably pharmaceutically acceptable. Suitable pharmaceutically acceptable salts can include acid addition salts or base addition salts. For a review of suitable pharmaceutically acceptable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent.

A pharmaceutically acceptable acid addition salt can be formed by reaction of a compound of formula (I) with a suitable inorganic or organic acid (such as hydrobromic, hydrochloric, sulphuric, nitric, phosphoric, succinic, maleic, acetic, propionic, fumaric, citric, tartaric, lactic, benzoic, salicylic, aspartic, p-toluenesulphonic, benzenesulphonic, methanesulphonic, ethanesulphonic, naphthalenesulphonic such as 2-naphthalenesulphonic, or hexanoic acid), optionally in a suitable solvent such as an organic solvent, to give the salt which is usually isolated for example by crystallisation and filtration or by evaporation followed by trituration. A pharmaceutically acceptable acid addition salt of a compound of formula (I) can comprise or be for example a hydrobromide, hydrochloride, sulfate, nitrate, phosphate, succinate, maleate, acetate, propionate, fumarate, citrate, tartrate, lactate, benzoate, salicylate, glutamate, aspartate, p-toluenesulphonate, benzenesulphonate, methanesulphonate, ethanesulphonate, naphthalenesulphonate (e.g. 2-naphthalenesulphonate) or hexanoate salt.

Other non-pharmaceutically acceptable salts, e.g. formates or trifluoroacetates, may be used, for example in the isolation of the compounds of formula (I), and are included within the scope of this invention.

The invention includes within its scope all possible stoichiometric and non-stoichiometric forms of the salts of the compounds of formula (I).

It will be appreciated from the foregoing that included within the scope of the invention are solvates, isomers and polymorphic forms of the compounds of formula (I) and salts thereof.

### STATEMENT OF THE INVENTION

In a first aspect there are provided compounds of formula (I):

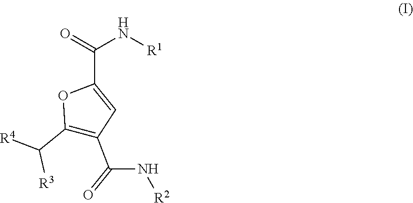

or a salt thereof
wherein:

R¹ is —C₁₋₃alkyl or cyclopropyl;

R² is —C₀₋₃alkyl-cycloalkyl, wherein the cycloalkyl group is optionally substituted with one, two or three R⁵ groups which may be the same or different; or

R² is —C₀₋₄alkyl-heterocyclyl or —(CH₂)ₚO-heterocyclyl wherein each heterocyclyl is optionally substituted by one or two R⁹ groups which may be the same or different; or

R² is H, —CH₃, —C₂₋₆alkyl optionally substituted by one, two, three, four or five fluoro, —C₂₋₆alkylOR⁶, —C₂₋₆alkylNR^{10a}R^{11a}, —(CH₂)ₘSO₂C₁₋₃alkyl, —(CH₂)ₘSO₂NR¹⁰R¹¹, —(CH₂)ₘC(O)NR¹⁰R¹¹, —(CH₂)ₘCN, —(CH₂)ₘCO₂R⁶, —(CH₂)ₘNHCO₂C₁₋₄alkyl, —(CH₂)ₘNHC(O)C₁₋₄alkyl or —(CH₂)ₙheteroaryl wherein the heteroaryl is optionally substituted by one or two R⁷ groups which may be the same or different;

R³ is H, —C₁₋₄alkyl, cyclopropyl, fluoro, chloro, —CH₂F, —C₀₋₃alkylOR⁵ or —C₀₋₃alkylCN;

R⁴ is phenyl or a heteroaryl group wherein each are optionally substituted by one, two or three R⁷ groups which may be the same or different;

each R⁵ is independently halo, —C₀₋₆alkyl-R⁸, —O—C₂₋₆alkyl-R⁸, —O—CH₂phenyl, —CN or —SO₂C₁₋₃alkyl;

R⁶ is H or —C₁₋₄alkyl;

each R⁷ is independently oxo, halo, —C₁₋₄alkyl optionally substituted by one, two or three fluoro, —C₀₋₃alkylOR⁶, —OC₂₋₃alkylOR⁶, —C₀₋₃alkylNR¹⁰R¹¹, —C₀₋₃alkyl-CONR¹⁰R¹¹, —CN, —SO₂—C₁₋₃alkyl, —SO₂NR¹⁰R¹¹ or —SO₂phenyl optionally substituted by —C₁₋₄alkyl

R⁸ is H, —OR⁶, —NR¹OR¹¹ or heteroaryl;

each R⁹ is independently halo, —C₁₋₄alkyl, cyclopropyl, cyclobutyl, —CH₂CF₃, —CH₂CHF₂, —CH₂CH₂F, —OCH₂CH₂OR⁶, —C₀₋₃alkylOR⁶, —C₀₋₃alkylNR¹⁰R¹¹, —NHCH₂CH₂OR⁶, —NHCO₂C₁₋₄alkyl, oxo, —C(O)R⁶, —C(O)OR⁶ or —C(O)NR¹OR¹¹;

R¹⁰ and R¹¹ are each independently selected from H and —C₁₋₃alkyl; or R¹⁰ and R¹¹ may join together with the nitrogen to which they are attached, to form a 4 to 7-membered heterocyclyl optionally substituted by one or two substituents independently selected from —C₁₋₃alkyl optionally substituted with one, two or three fluorine atoms, —C₂₋₄alkylOH, —OH and F;

R^{10a} and R^{11a} are each independently selected from H and —C₁₋₃alkyl;

m is an integer selected from 2, 3 or 4;

n is an integer selected from 0, 1, 2, 3 or 4; and

p is an integer selected from 2, 3 or 4.

In one embodiment R¹ is methyl.

In one embodiment R² is —C₀₋₃alkyl-cycloalkyl, wherein the cycloalkyl group is optionally substituted with one, two or three R⁵ groups which may be the same or different. In one embodiment R² is a —C₀₋₃alkyl-C₃₋₇cycloalkyl group, wherein the C₃₋₇cycloalkyl group is selected from the group consisting of cyclopropyl, cyclobutyl, cyclohexyl or bicyclo[3.1.0]hexanyl, said groups being optionally substituted with one, two or three R⁵ groups which may be the same or different. In another embodiment R² is cyclopropyl, cyclobutyl, cyclohexyl or bicyclo[3.1.0]hexanyl optionally substituted with one, two or three R⁵ groups which may be the same or different. In another embodiment R² is cyclopropyl, cyclobutyl, cyclohexyl or bicyclo[3.1.0]hexanyl optionally substituted with one R⁵ group selected from methyl, fluoro and —OH. In a particular embodiment R² is a cyclopropyl optionally substituted by one methyl group. In another particular embodiment R² is a cyclohexyl group optionally substituted with a OH group. In another embodiment R² is a cyclohexyl group optionally substituted with a methoxy group.

In one embodiment R² is —C₀₋₄alkyl-heterocyclyl or —(CH₂)ₚO-heterocyclyl wherein each heterocyclyl is optionally substituted by one or two R⁹ groups which may be the same or different. In another embodiment R² is —C₀₋₄alkyl-heterocyclyl wherein the heterocyclyl is optionally substituted by one or two R⁹ groups which may be the same or different. In another embodiment R² is —C₀₋₄alkyl-heterocyclyl which is -heterocyclyl, —CH₂CH₂-heterocyclyl or —CH₂CH₂CH₂-heterocyclyl. In another embodiment R² is —C₀₋₄alkyl-heterocyclyl wherein the heterocyclyl is selected from oxetanyl, tetrahydrofuranyl, tetrahydro-2H-pyranyl, morpholinyl, piperidinyl, piperazinyl, (1r,5s)-3-oxabicyclo[3.1.0]hexanyl and (1r,5s)-3-azabicyclo[3.1.0]hexanyl said groups being optionally substituted by one or two R⁹ groups which may be the same or different. In another embodiment R² is —C₀₋₄alkyl-heterocyclyl wherein the heterocyclyl is selected from oxetanyl, tetrahydrofuranyl, tetrahydro-2H-pyranyl, morpholinyl, piperidinyl, piperazinyl, (1r,5s)-3-oxabicyclo[3.1.0]hexanyl and (1r,5s)-3-azabicyclo[3.1.0]hexanyl optionally substituted by one or two R⁹ groups selected from methyl, —C(O)CH₃ and fluoro. In a further embodiment R² is —C₀₋₄alkyl-heterocyclyl wherein the heterocyclyl optionally substituted by one or two R⁹ groups, is selected from:

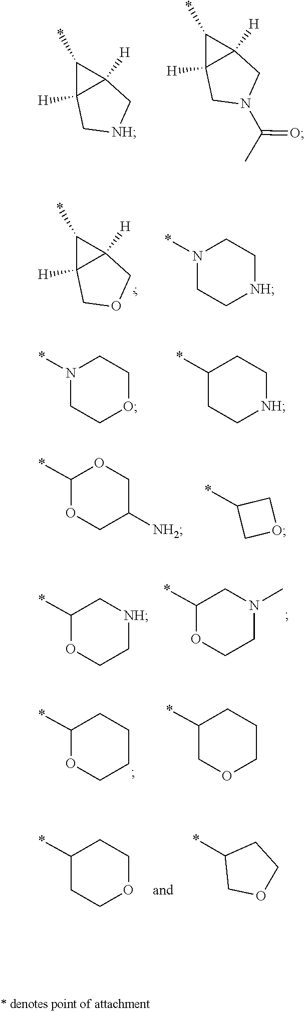

In another embodiment R² is H, —CH₃, C₂₋₆alkyl optionally substituted by one, two, three, four or five fluoro, —C₂₋₆alkylOR⁶, —C₂₋₆alkylNR¹⁰R^{11a}, —(CH₂)ₘSO₂C₁₋₃alkyl, —(CH₂)ₘSO₂NR¹⁰R¹¹, —(CH₂)ₘC(O)NR¹⁰R¹¹, —(CH₂)ₘCN, —(CH₂)ₘCO₂R⁶, —(CH₂)ₘNHCO₂C₁₋₄alkyl —(CH₂)ₘNHC(O)C₁₋₄alkyl or —(CH₂)ₙheteroaryl wherein the heteroaryl is optionally substituted by one or two R⁷ groups which may be the same or different.

In another embodiment R² is selected from methyl, ethyl, propyl, iso-propyl, butyl, —CH₂CH₂CH(CH₃)₂, —CH₂CH(CH₃)₂, —CH₂CH₂OR⁶, —CH₂CH₂CH₂OR⁶, —CH₂CH(CH₃)OR⁶, —CH₂CH₂CH(CH₃)OR⁶, —CH₂CH₂CH(CH₃)NR^{10a}R^{11a}, —CH₂CH₂CH₂NR^{10a}R^{11a}, —(CH₂)ₘSO₂CH₃, —(CH₂)ₘC(O)NHCH₃, —(CH₂)ₘCN, —(CH₂)ₘCO₂R⁶, —(CH₂)ₘCF₃ and —(CH₂)ₘNHCO₂C(CH₃)₃. In another embodiment R² is methyl or is —C₂₋₆alkyl selected from ethyl, propyl, iso-propyl, butyl, —CH₂CH₂CH(CH₃)₂ and —CH₂CH(CH₃)₂. In another embodiment R² is —C₂₋₆alkylOR⁶ selected from —CH₂CH₂OR⁶, —CH₂CH₂CH₂OR⁶, —CH₂CH(CH₃)OR⁶ and —CH₂CH₂CH(CH₃)OR⁶. In another embodiment R² is —C₂₋₆alkylNR¹⁰R¹¹ selected from —CH₂CH₂CH(CH₃)NR^{10a}R^{11a} and —CH₂CH₂CH₂NR^{10a}R^{11a}. In another embodiment R² is —(CH₂)ₘSO₂CH₃. In another embodiment R² is —(CH₂)ₘC(O)NHCH₃. In another embodiment R² is —(CH₂)ₘCN. In another embodiment R² is —(CH₂)ₘCO₂R⁶. In another embodiment R² is —(CH₂)ₘCF₃. In another embodiment R² is —(CH₂)ₘNHCO₂C(CH₃)₃.

In one embodiment R² is —(CH₂)ₙheteroaryl wherein the heteroaryl is optionally substituted by one or two R⁷ groups which may be the same or different. In a further embodiment R² is —(CH₂)ₙC₅₋₆heteroaryl wherein the C₅₋₆heteroaryl is selected from furanyl, thienyl, pyrrolyl, triazolyl, thiazolyl, oxazolyl, isoxazolyl, oxadiazolyl, imidazolyl, pyrazolyl, triazolyl, tetrazolyl, isoxazolyl, pyridinyl, pyridazinyl, pyrazinyl and pyrimidinyl said groups being optionally substituted by one or two R⁷ substituents independently selected from halo, C₁₋₄alkyl (such as methyl) and —C₀₋₃alkylOR⁶. In another embodiment there is provided compounds of formula (I) in which R² is —(CH₂)ₙC₅₋₆heteroaryl wherein the C₅₋₆heteroaryl is pyrazolyl optionally substituted by C₁₋₄alkyl or —C₀₋₃alkylOR⁶. In a particular embodiment there is provided compounds of formula (I) in which R² is —(CH₂)ₙC₅₋₆heteroaryl wherein the optionally substituted C₅₋₆heteroaryl group is selected from the group consisting of

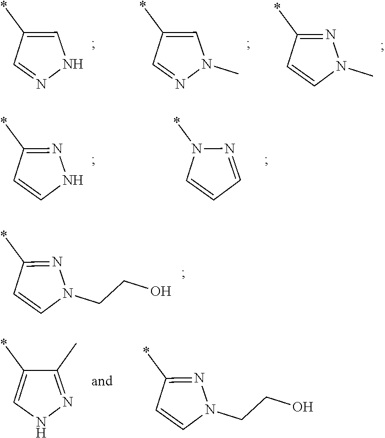

wherein * denotes the point of attachment.

In one embodiment R³ is H, methyl, ethyl, —CH₂F, —CH₂OH, —CH(OH)CH₃, —OMe or —CH₂CN. In one embodiment R³ is H, methyl, —CH₂OH, —OMe or —CH₂CN.

In one embodiment R⁴ is phenyl optionally substituted by one, two or three R⁷ groups which may be the same or different. In another embodiment R⁴ is unsubstituted phenyl. In another embodiment R⁴ is phenyl substituted by one or two R⁷ groups which may be the same or different selected from halo, —C₁₋₄alkyl optionally substituted one, two or three fluoro, —C₀₋₃alkylOR⁶, —OC₂₋₃alkylOR⁶ and —CN. In another embodiment R⁴ is phenyl substituted by one or two R⁷ groups which may be the same or different selected from halo, —C₁₋₄alkyl, —C₀₋₃alkylOR⁶ and —CN. In another embodiment R⁴ is phenyl substituted by one R⁷ groups selected from the group consisting of fluoro, chloro, methyl, cyano and methoxy.

In another embodiment R⁴ is a heteroaryl group which is pyridyl optionally substituted by one, two or three R⁷ groups which may be the same or different. In another embodiment R⁴ is a heteroaryl group which is unsubstituted pyridyl. In another embodiment R⁴ is a heteroaryl group which is pyridyl substituted by one methyl group.

In another embodiment R⁴ is a heteroaryl group which is indolyl (e.g 1H-indol-4-yl or 1H-indol-5-yl) optionally substituted by one, two or three R⁷ groups which may be the same or different. In another embodiment R⁴ is a heteroaryl group which is 1H-indol-4-yl.

In another embodiment R⁴ is a heteroaryl group which is a pyrrolopyridinyl (e.g. 1H-pyrrolo[2,3,b]pyridinyl or 1H-pyrrolo[2,3,c]pyridinyl)) optionally substituted by one, two or three R⁷ groups which may be the same or different. In another embodiment R⁴ is a heteroaryl group which is unsubstituted pyrrolopyridinyl.

In one embodiment each R⁵ is independently halo or —C₀₋₆alkyl-R⁸ wherein R⁸ is H, OR⁶ (such as OH) or NR¹⁰R¹¹ (such as NH₂).

In one embodiment R⁶ is H, methyl, ethyl or t-butyl.

In one embodiment each R⁷ is independently oxo, halo, —C₁₋₄alkyl optionally substituted by one, two or three fluoro, —C₀₋₃alkylOR⁶, —C₀₋₃alkylNR¹⁰R¹¹, —C₀₋₃alkyl-CONR¹⁰R¹¹, —CN, —SO₂—C₁₋₃alkyl or —SO₂NR¹⁰R¹¹. In another embodiment each R⁷ is independently halo, —C₁₋₄alkyl optionally substituted by one, two or three fluoro, —C₀₋₃alkylOR⁶ or CN.

In one embodiment each R⁷ is independently halo, —C₁₋₄alkyl, —C₀₋₃alkylOR⁶ or —CN; In one embodiment R⁸ is H, —OH or methoxy.

In one embodiment each R⁹ is independently halo, C₁₋₄alkyl (such as methyl), —C₀₋₃alkylOR⁶, —C₀₋₃alkylNR¹⁰R¹¹, oxo, or —C(O)R⁶ (such as C(O)CH₃).

In one embodiment m is 2 or 3.

In one embodiment n is 0, 1 or 2. In a further embodiment n is 0. In a yet further embodiment n is 2.

In one embodiment p is 2 or 3.

It is to be understood that the present invention covers all combinations of substituent groups described hereinabove.

Compounds of the invention include the compounds of Examples 1 to 110 and salts thereof.

Compounds of the invention include the compounds of Examples 1 to 111 and salts thereof.

In one embodiment the compound of formula (I) is selected from:
N⁴-((1R,3R,5S,6r)-3-hydroxybicyclo[3.1.0]hexan-6-yl)-N²-methyl-5-((S)-1-phenylethyl)furan-2,4-dicarboxamide;
5-((S*)-1-(3-chlorophenyl)ethyl)-N⁴-((1r,4S)-4-hydroxycyclohexyl)-N²-methylfuran-2,4-dicarboxamide;
N⁴-((1r,4S)-4-Hydroxycyclohexyl)-N²-methyl-5-((S)-1-phenylethyl)furan-2,4-dicarboxamide; and
N⁴-((1R,5S,6r)-3-oxabicyclo[3.1.0]hexan-6-yl)-N²-methyl-5-((S)-1-phenylethyl)furan-2,4-dicarboxamide
   or a salt thereof.

In a second aspect of the present invention, there is provided a pharmaceutical composition comprising a compound of formula (I) or a pharmaceutically acceptable salt thereof and one or more pharmaceutically acceptable excipients.

In a third aspect of the present invention, there is provided a compound of formula (I), or a pharmaceutically acceptable salt thereof for use in therapy, in particular in the treatment of diseases or conditions for which a bromodomain inhibitor is indicated.

In a fourth aspect of the present invention, there is provided a method of treating diseases or conditions for which a bromodomain inhibitor is indicated in a subject in need thereof which comprises administering a therapeutically effective amount of a compound of formula (I) or a pharmaceutically acceptable salt thereof.

In a fifth aspect of the present invention, there is provided the use of a compound of formula (I), or a pharmaceutically acceptable salt thereof in the manufacture of a medicament for the treatment of diseases or conditions for which a bromodomain inhibitor is indicated.

The compounds of the invention may possess an improved profile over known BET inhibitors (including properties such as potency, selectivity and/or developability). Certain compounds of the invention may have an advantageous combination of such properties.

### STATEMENT OF USE

The compounds of formula (I) and salts thereof are bromodomain inhibitors, and thus are believed to have potential utility in the treatment of diseases or conditions for which a bromodomain inhibitor is indicated.

Bromodomain inhibitors are believed to be useful in the treatment of a variety of diseases or conditions related to systemic or tissue inflammation, inflammatory responses to infection or hypoxia, cellular activation and proliferation, lipid metabolism, fibrosis and in the prevention and treatment of viral infections.

Bromodomain inhibitors may be useful in the treatment of a wide variety of acute or chronic autoimmune and/or inflammatory conditions such as rheumatoid arthritis, psoriatic arthritis, ankylosing spondylitis, osteoarthritis, acute gout, psoriasis, systemic lupus erythematosus, multiple sclerosis, inflammatory bowel disease (Crohn's disease and ulcerative colitis), asthma, chronic obstructive airways disease, pneumonitis, myocarditis, pericarditis, myositis, eczema, dermatitis (including atopic dermatitis), alopecia, vitiligo, bullous skin diseases, nephritis, vasculitis, hypercholesterolemia, atherosclerosis, Alzheimer's disease, Sjögren's syndrome, sialoadenitis, central retinal vein occlusion, branched retinal vein occlusion, Irvine-Gass syndrome (post cataract and post-surgical), retinitis pigmentosa, pars planitis, birdshot retinochoroidopathy, epiretinal membrane, cystic macular edema, parafoveal telengiectasis, tractional maculopathies, vitreomacular traction syndromes, retinal detachment, neuroretinitis, idiopathic macular edema, retinitis, dry eye (keratoconjunctivitis Sicca), vernal keratoconjunctivitis, atopic keratoconjunctivitis, uveitis (such as anterior uveitis, pan uveitis, posterior uveitis, uveitis-associated macular edema), scleritis, diabetic retinopathy, diabetic macula edema, age-related macular dystrophy, hepatitis, pancreatitis, primary biliary cirrhosis, sclerosing cholangitis, acute alcoholic hepatitis, chronic alcoholic hepatitis, alcoholic steato-hepatitis, non-alcoholic steato-hepatitis (NASH), cirrhosis, Childs-Pugh cirrhosis, autoimmune hepatitis, fulminant hepatitis, chronic viral hepatitis, alcoholic liver disease, systemic sclerosis, systemic sclerosis with associated interstitial lung disease, sarcoidosis, neurosarcoidosis, Addison's disease, hypophysitis, thyroiditis, Type I diabetes, Type II diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, pyoderma gangrenosum, vasculitis with organ involvement, acute rejection of transplanted organs and systemic sclerosis.

In one embodiment the acute or chronic autoimmune and/or inflammatory condition is a disorder of lipid metabolism mediated via the regulation of APO-A1 such as hypercholesterolemia, atherosclerosis or Alzheimer's disease.

In another embodiment the acute or chronic autoimmune and/or inflammatory condition is a respiratory disorder such as asthma or chronic obstructive airways disease.

In another embodiment the acute or chronic autoimmune and/or inflammatory condition is a systemic inflammatory disorder such as rheumatoid arthritis, osteoarthritis, acute gout, psoriasis, systemic lupus erythematosus, multiple sclerosis or inflammatory bowel disease (Crohn's disease or Ulcerative colitis).

In another embodiment, the acute or chronic autoimmune and/or inflammatory condition is multiple sclerosis.

In another embodiment, the acute or chronic autoimmune and/or inflammatory condition is Type I diabetes.

In another embodiment, the acute or chronic autoimmune and/or inflammatory condition is rheumatoid arthritis.

Bromodomain inhibitors may be useful in the treatment of depression.

Bromodomain inhibitors may be useful in the treatment of diseases or conditions which involve inflammatory responses to infections with bacteria, viruses, fungi, parasites or their toxins, such as sepsis, acute sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus. In one embodiment the disease or condition which involves an inflammatory response to an infection with bacteria, a virus, fungi, a parasite or their toxins is acute sepsis.

Bromodomain inhibitors may be useful in the treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

Bromodomain inhibitors may be useful in the treatment of cardiovascular diseases such as coronary artery diseases (for example, angina or myocardial infarction), pulmonary arterial hypertension, cerebro-vascular ischaemia (stroke), hypertensive heart disease, rheumatic heart disease, cardiomyopathy, atrial fibrillation, congenital heart disease, endocarditis, aortic aneurysms or peripheral artery disease.

Bromodomain inhibitors may be useful in the treatment of fibrotic conditions such as idiopathic pulmonary fibrosis, pulmonary fibrosis, cystic fibrosis, progressive massive fibrosis, renal fibrosis, liver fibrosis, liver cirrhosis, non-alcoholic steatohepatitis (NASH), non-alcoholic fatty liver disease (NAFLD), post-operative stricture, keloid scar formation, scleroderma (including morphea and systemic sclerosis), cardiac fibrosis, atrial fibrosis, endomyocardial fibrosis, old myocardial infarction, arthrofibrosis, Dupuytren's contracture, mediastinal, myelofibrosis, Peyronie's disease, nephrogenic systemic fibrosis, retroperitoneal fibrosis and adhesive capsulitis.

Bromodomain inhibitors may be useful in the treatment of viral infections such as herpes simplex infections and reactivations, cold sores, herpes zoster infections and reactivations, chickenpox, shingles, human papilloma virus (HPV), human immunodeficiency virus (HIV), cervical neoplasia, adenovirus infections, including acute respiratory disease, poxvirus infections such as cowpox or smallpox, or African swine fever virus. In one embodiment the viral infection is a HPV infection of skin or cervical epithelia. In another embodiment the viral infection is a latent HIV infection.

Bromodomain inhibitors may be useful in the treatment of a wide variety of bone disorders such as osteoporosis, osteopenia, osteoarthritis and ankylosing spondylitis.

Bromodomain inhibitors may be useful in the treatment of cancer, including hematological cancers (such as leukaemia, lymphoma and multiple myeloma), epithelial cancers (including lung, breast or colon carcinomas), midline carcinomas, or mesenchymal, hepatic, renal or neurological tumours.

Bromodomain inhibitors may be useful in the treatment of one or more cancers selected from brain cancer (gliomas), glioblastomas, Bannayan-Zonana syndrome, Cowden disease, Lhermitte-Duclos disease, breast cancer, inflammatory breast cancer, colorectal cancer, Wilm's tumor, Ewing's sarcoma, rhabdomyosarcoma, ependymoma, medulloblastoma, colon cancer, head and neck cancer, kidney cancer, lung cancer, liver cancer, melanoma, squamous cell carcinoma, ovarian cancer, pancreatic cancer, prostate cancer, sarcoma cancer, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukaemia, erythroleukemia, malignant lymphoma, Hodgkins lymphoma, non-Hodgkins lymphoma, lymphoblastic T-cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), NUT-midline carcinoma and testicular cancer.

In one embodiment the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is breast cancer. In another embodiment the cancer is colorectal cancer. In another embodiment the cancer is prostate cancer. In another embodiment the cancer is castration resistant prostate cancer.

Bromodomain inhibitors may be useful in the treatment of diseases associated with systemic inflammatory response syndrome, such as sepsis, burns, pancreatitis, major trauma, haemorrhage and ischaemia. In this embodiment, the bromodomain inhibitor would be administered at the point of diagnosis to reduce the incidence of: SIRS, the onset of shock, multi-organ dysfunction syndrome, which includes the onset of acute lung injury, ARDS, acute renal, hepatic, cardiac or gastro-intestinal injury and mortality. In another embodiment the bromodomain inhibitor would be administered prior to surgical or other procedures associated with a high risk of sepsis, haemorrhage, extensive tissue damage, SIRS or MODS (multiple organ dysfunction syndrome). In a particular embodiment the disease or condition for which a bromodomain inhibitor is indicated is sepsis, sepsis syndrome, septic shock and endotoxaemia. In another embodiment, the bromodomain inhibitor is indicated for the treatment of acute or chronic pancreatitis. In another embodiment the bromodomain is indicated for the treatment of burns.

The present invention thus provides a compound of formula (I) or a pharmaceutically acceptable salt thereof for use in therapy.

The compound of formula (I) or a pharmaceutically salt thereof can be used in the treatment of diseases or conditions for which a bromodomain inhibitor is indicated. The present invention thus provides a compound of formula (I) or a pharmaceutically acceptable salt thereof for use in the treatment of a disease or condition for which a bromodomain inhibitor is indicated. In one embodiment there is provided a compound of formula (I) or a pharmaceutically acceptable salt thereof for use in the treatment of acute or chronic auto-immune and/or inflammatory conditions. In one embodiment there is provided a compound of formula (I) or a pharmaceutically acceptable salt thereof for use in the treatment of rheumatoid arthritis. In another embodiment there is provided a compound of formula (I) or a pharmaceutically acceptable salt thereof for use in the treatment of diseases or conditions which involve inflammatory responses to infections with bacteria, viruses, fungi, parasites or their toxins. In another embodiment there is provided a compound of formula (I) or a pharmaceutically acceptable salt thereof for use in the treatment of conditions associated with ischaemia-reperfusion injury. In another embodiment there is provided a compound of formula (I) or a pharmaceutically acceptable salt thereof for use in the treatment of cardiovascular diseases. In another embodiment there is provided a compound of formula (I) or a pharmaceutically acceptable salt thereof for use in the treatment of fibrotic conditions. In another embodiment there is provided a compound of formula (I) or a pharmaceutically acceptable salt thereof for use in the treatment of viral infections. In another embodiment there is provided a compound of formula (I) or a pharmaceutically acceptable salt thereof for use in the treatment of bone disorders. In another embodiment there is provided a compound of formula (I) or a pharmaceutically acceptable salt thereof for use in the treatment of cancer. In a further embodiment there is provided a compound of formula (I) or a pharmaceutically acceptable salt thereof for use in the treatment of diseases associated with systemic inflammatory response syndrome.

Also provided is the use of a compound of formula (I) or a pharmaceutically acceptable salt thereof in the manufacture of a medicament for the treatment of diseases or conditions for which a bromodomain inhibitor is indicated. In one embodiment there is provided the use of a compound of formula (I) or a pharmaceutically acceptable salt thereof in the manufacture of a medicament for the treatment of acute or chronic auto-immune and/or inflammatory conditions. In one embodiment there is provided the use of a compound of formula (I) or a pharmaceutically acceptable salt thereof in the manufacture of a medicament for the treatment of rheumatoid arthritis. In another embodiment there is provided the use of a compound of formula (I) or a pharmaceutically acceptable salt thereof in the manufacture of a medicament for the treatment of diseases or conditions which involve inflammatory responses to infections with bacteria, viruses, fungi, parasites or their toxins. In another embodiment there is provided the use of a compound of formula (I) or a pharmaceutically acceptable salt thereof in the manufacture of a medicament for the treatment of conditions associated with ischaemia-reperfusion injury. In another embodiment there is provided the use of a compound of formula (I) or a pharmaceutically acceptable salt thereof in the manufacture of a medicament for the treatment of cardiovascular diseases. In another embodiment there is provided the use of a compound of formula (I) or a pharmaceutically acceptable salt thereof in the manufacture of a medicament for the treatment of fibrotic conditions. In another embodiment there is provided the use of a compound of formula (I) or a pharmaceutically acceptable salt thereof in the manufacture of a medicament for the treatment of viral infections. In another embodiment there is provided the use of a compound of formula (I) or a pharmaceutically acceptable salt thereof in the manufacture of a medicament for the treatment of cancer. In a further embodiment there is provided the use of a compound of formula (I) or a pharmaceutically acceptable salt thereof in the manufacture of a medicament for the treatment of diseases associated with systemic inflammatory response syndrome.

Also provided is a method of treating diseases or conditions for which a bromodomain inhibitor is indicated in a subject in need thereof which comprises administering a therapeutically effective amount of compound of formula (I) or a pharmaceutically acceptable salt thereof. In one embodiment there is provided a method of treating acute or chronic auto-immune and/or inflammatory conditions in a subject in need thereof which comprises administering a therapeutically effective amount of a compound of formula (I) or a pharmaceutically acceptable salt thereof. In one embodiment there is provided a method of treating rheumatoid arthritis in a subject in need thereof which comprises administering a therapeutically effective amount of a compound of formula (I) or a pharmaceutically acceptable salt thereof. In another embodiment there is provided a method of treating diseases or conditions which involve inflammatory responses to infections with bacteria, viruses, fungi, parasites or their toxins in a subject in need thereof which comprises administering a therapeutically effective amount of a compound of formula (I) or a pharmaceutically acceptable salt thereof. In another embodiment there is provided a method of treating conditions associated with ischaemia-reperfusion injury in a subject in need thereof which comprises administering a therapeutically effective amount of a compound of formula (I) or a pharmaceutically acceptable salt thereof. In another embodiment there is provided a method of treating cardiovascular diseases in a subject in need thereof which comprises administering a therapeutically effective amount of a compound of formula (I) or a pharmaceutically acceptable salt thereof. In another embodiment there is provided a method of treating fibrotic conditions in a subject in need thereof which comprises administering a therapeutically effective amount of a compound of formula (I) or a pharmaceutically acceptable salt thereof. In another embodiment there is provided a method of treating viral infections in a subject in need thereof which comprises administering a therapeutically effective amount of a compound of formula (I) or a pharmaceutically acceptable salt thereof. In another embodiment there is provided a method of treating cancer in a subject in need thereof which comprises administering a therapeutically effective amount of a compound of formula (I) or a pharmaceutically acceptable salt thereof. In a further embodiment there is provided a method of treating diseases associated with systemic inflammatory response syndrome in a subject in need thereof which comprises administering a therapeutically effective amount of a compound of formula (I) or a pharmaceutically acceptable salt thereof.

Suitably the subject in need thereof is a mammal, particularly a human.

The invention further provides for a method for inhibiting a bromodomain containing protein which comprises contacting the bromodomain containing protein with a compound of formula (I) or a pharmaceutically acceptable salt thereof.

As used herein the reference to the “treatment” of a particular disease or condition includes the prevention or prophylaxis of such a disease or condition.

### Pharmaceutical Compositions/Routes of Administration/Dosages

### Compositions

While it is possible that for use in therapy, a compound of formula (I) as well as pharmaceutically acceptable salts thereof may be administered as the raw chemical, it is common to present the active ingredient as a pharmaceutical composition. The compounds of formula (I) and pharmaceutically acceptable salts thereof will normally, but not necessarily, be formulated into pharmaceutical compositions prior to administration to a patient. Accordingly, in another aspect there is provided a pharmaceutical composition comprising a compound of formula (I), or a pharmaceutically acceptable salt thereof, and one or more (e.g. two, three, four, five or six) pharmaceutically acceptable excipients. The compounds of formula (I) and pharmaceutically acceptable salts are as described above. The excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of formula (I), or a pharmaceutically acceptable salt thereof, with one or more pharmaceutically acceptable excipients. A pharmaceutical composition comprising a compound of formula (I) or a pharmaceutically acceptable salt thereof may be prepared by, for example, admixture at ambient temperature and atmospheric pressure. The pharmaceutical composition can be used in the treatment of any of the conditions described herein.

In a further aspect the invention is directed to pharmaceutical compositions for the treatment of a disease or condition for which a bromodomain inhibitor is indicated comprising a compound of formula (I) or a pharmaceutically acceptable salt thereof.

Since the compounds of formula (I) are intended for use in pharmaceutical compositions it will be readily understood that they are each preferably provided in substantially pure form, for example, at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), ocular (including topical, intraocular, subconjunctival, episcleral, sub-Tenon), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

The pharmaceutical compositions of the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions of the invention may be prepared and packaged in unit dosage form wherein each physically discrete unit contains a compound of formula (I) or a pharmaceutically acceptable salt thereof. When prepared in unit dosage form, the pharmaceutical compositions of the invention typically may contain, for example, from 0.25 mg to 1 g, or from 0.5 mg to 500 mg, or from 1 mg to 100 mg, of a compound of formula (I) or a pharmaceutically acceptable salt thereof.

The compound of formula (I) or a pharmaceutically acceptable salt thereof and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of formula (I) or pharmaceutically acceptable salts thereof once administered to the subject from one organ, or portion of the body, to another organ, or portion of the body. Certain pharmaceutically acceptable excipients may be chosen for their ability to enhance subject compliance.

Suitable pharmaceutically-acceptable excipients include the following types of excipients: carriers, diluents, fillers, binders, disintegrants, lubricants, glidants, granulating agents, coating agents, wetting agents, solvents, co-solvents, suspending agents, emulsifiers, sweetners, flavouring agents, flavour-masking agents, colouring agents, anti-caking agents, humectants, chelating agents, plasticisers, viscosity increasing agents, antioxidants, preservatives, stabilisers, surfactants, and buffering agents. The skilled artisan will appreciate that certain pharmaceutically-acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one embodiment the pharmaceutical composition is adapted for parenteral administration, particularly intravenous administration.

In one embodiment the pharmaceutical composition is adapted for oral administration.

In one embodiment the pharmaceutical composition is adapted for topical administration.

Pharmaceutical compositions adapted for parenteral administration include aqueous and non-aqueous sterile injection solutions (which may contain anti-oxidants, buffers, bacteriostats and solutes which render the composition isotonic with the blood of the intended recipient) and aqueous and non-aqueous sterile suspensions (which may include suspending agents and thickening agents). The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine size (e.g. by micronisation) and mixing with a similarly prepared pharmaceutical carrier such as an edible carbohydrate, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents (disintegrants) and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrants include starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of formula (I) and pharmaceutically acceptable salts thereof can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Compositions for oral administration may be designed to provide a modified release profile so as to sustain or otherwise control the release of the therapeutically active agent.

Where appropriate, dosage unit compositions for oral administration can be microencapsulated. The composition may be prepared to prolong or sustain the release as for example by coating or embedding particulate material in polymers, wax or the like.

For compositions suitable and/or adapted for oral administration, the compound of formula (I) or a pharmaceutically acceptable salt thereof, may be in a particle-size-reduced form e.g. obtained by micronisation. The preferable particle size of the size-reduced (e.g. micronised) compound or salt is defined by a D₅₀ value of about 0.5 to about 10 microns (for example as measured using laser diffraction).

The compounds of formula (I) and pharmaceutically acceptable salts thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for topical administration may be formulated as ointments, creams, suspensions, emulsions, lotions, powders, solutions, pastes, gels, foams, sprays, aerosols or oils. Such pharmaceutical compositions may include conventional additives which include, but are not limited to, preservatives, solvents to assist drug penetration, co-solvents, emollients, propellants, viscosity modifying agents (gelling agents), surfactants and carriers. In one embodiment there is provided a pharmaceutical composition adapted for topical administration which comprises between 0.01-10%, or between 0.01-1% of the compound of formula (I), or a pharmaceutically acceptable salt thereof, by weight of the composition.

For treatments of the eye or other external tissues, for example mouth and skin, the compositions are preferably applied as a topical ointment, cream, gel, spray or foam. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent. Compositions to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

Dosage forms for nasal or inhaled administration may conveniently be formulated as aerosols, solutions, suspensions, gels or dry powders.

For compositions suitable and/or adapted for inhaled administration, it is preferred that the compound of formula (I) or a pharmaceutically acceptable salt thereof, is in a particle-size-reduced form e.g. obtained by micronisation. The preferable particle size of the size-reduced (e.g. micronised) compound or salt is defined by a D₅₀ value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the compound of formula (I) or a pharmaceutically acceptable salt thereof (preferably in particle-size-reduced form, e.g. in micronised form), and optionally a performance modifier such as L-leucine or another amino acid and/or metal salt of stearic acid such as magnesium or calcium stearate. Preferably, the dry powder inhalable composition comprises a dry powder blend of lactose e.g. lactose monohydrate and the compound of formula (I) or salt thereof. Such compositions can be administered to the patient using a suitable device such as the DISKUS® device, marketed by GlaxoSmithKline which is for example described in GB 2242134 A.

The compounds of formula (I) and pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in International Patent Application Publication No. WO 2005/044354 A1.

A therapeutically effective amount of a compound of formula (I) or a pharmaceutically acceptable salt thereof, will depend upon a number of factors including, for example, the age and weight of the patient, the precise condition requiring treatment and its severity, the nature of the formulation, and the route of administration, and will ultimately be at the discretion of the attendant physician or veterinarian. In the pharmaceutical composition, each dosage unit for oral or parenteral administration preferably contains from 0.01 mg to 3000 mg, more preferably 0.5 mg to 1000 mg, of a compound of formula (I) or a pharmaceutically acceptable salt thereof, calculated as the free base. Each dosage unit for nasal or inhaled administration preferably contains from 0.001 mg to 50 mg, more preferably 0.01 mg to 5 mg, of a compound of the formula (I) or a pharmaceutically acceptable salt thereof, calculated as the free base.

The pharmaceutically acceptable compounds of formula (I) and pharmaceutically acceptable salts thereof, can be administered in a daily dose (for an adult patient) of, for example, an oral or parenteral dose of 0.01 mg to 3000 mg per day, 0.5 mg to 1000 mg per day or 100 mg to 2500 mg per day, or a nasal or inhaled dose of 0.001 mg to 50 mg per day or 0.01 mg to 5 mg per day, of the compound of the formula (I) or a pharmaceutically acceptable salt thereof, calculated as the free base. This amount may be given in a single dose per day or more usually in a number (such as two, three, four, five or six) of sub-doses per day such that the total daily dose is the same. An effective amount of a salt thereof, may be determined as a proportion of the effective amount of the compound of formula (I) per se.

The compounds of formula (I) and pharmaceutically acceptable salts thereof may be employed alone or in combination with other therapeutic agents. Combination therapies according to the present invention thus comprise the administration of at least one compound of formula (I) or a pharmaceutically acceptable salt thereof, and the use of at least one other therapeutically active agent. The compound(s) of formula (I) and pharmaceutically acceptable salts thereof, and the other therapeutically active agent(s) may be administered together in a single pharmaceutical composition or separately and, when administered separately this may occur simultaneously or sequentially in any order. The amounts of the compound(s) of formula (I) and pharmaceutically acceptable salts thereof, and the other therapeutically active agent(s) and the relative timings of administration will be selected in order to achieve the desired combined therapeutic effect. Thus in a further aspect, there is provided a combination product comprising a compound of formula (I) or a pharmaceutically acceptable salt thereof, together with one or more other therapeutically active agents.

Thus in one aspect, the compound of formula (I) or a pharmaceutically acceptable salt thereof, and pharmaceutical compositions comprising a compound of formula (I) or a pharmaceutically acceptable salt thereof, according to the invention may be used in combination with or include one or more other therapeutic agents, for example selected from antibiotics, anti-virals, glucocorticosteroids, muscarinic antagonists, beta-2 agonists and Vitamin D3 analogues. In a further embodiment a compound of formula (I) or a pharmaceutically acceptable salt thereof may be used in combination with a further therapeutic agent which is suitable for the treatment of cancer. Examples of such further therapeutic agents are described in Cancer Principles and Practice of Oncology by V. T. Devita and S. Hellman (editors), 6^{th} edition (2001), Lippincott Williams & Wilkins Publishers. A person of ordinary skill in the art would be able to discern which combinations of agents would be useful based on the particular characteristics of the drugs and the cancer involved. Further therapeutic agents to be used in combination with the compound of formula (I) or a pharmaceutically acceptable salt thereof include, but are not limited to, anti-microtubule agents (such as diterpenoids and vinca alkaloids); platinum coordination complexes; alkylating agents (such as nitrogen mustards, oxazaphosphorines, alkylsulphonates, nitrosoureas, and triazenes); antibiotic agents (such as anthracyclins, actinomycins and bleomycins); topoisomerase II inhibitors (such as epipodophyllotoxins); antimetabolites (such as purine and pyrimidine analogues and anti-folate compounds); topoisomerase I inhibitors (such as camptothecins; hormones and hormonal analogues); signal transduction pathway inhibitors (such as tyropsine receptor inhibitors); non-receptor tyrosine kinase angiogenesis inhibitors; immunotherapeutic agents (such as PD-1 inhibitors, including nivolumab and pembrolizumab, and CTLA-4 inhibitors, including ipilimumab); proapoptotic agents; epigenetic or transcriptional modulators (such as histone deacetylase inhibitors) and cell cycle signaling inhibitors.

It will be appreciated that when the compound of formula (I) or a pharmaceutically acceptable salt thereof, is administered in combination with other therapeutic agents normally administered by the inhaled, intravenous, oral or intranasal route, that the resultant pharmaceutical composition may be administered by the same routes. Alternatively the individual components of the composition may be administered by different routes.

It will be clear to a person skilled in the art that, where appropriate, the other therapeutic agent(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic agent. It will be clear also that, where appropriate, the therapeutic agents may be used in optically pure form.

The combinations referred to above may conveniently be presented for use in the form of a pharmaceutical composition and thus pharmaceutical compositions comprising a combination as defined above together with a pharmaceutically acceptable excipient represent a further aspect of the invention.

### General Synthetic Routes

The compounds of the invention may be made by a variety of methods. Any previously defined variable will continue to have the previously defined meaning unless otherwise indicated. Illustrative general synthetic methods are set out in the following schemes, and can be readily adapted to prepare other compounds of the invention. Specific compounds of the invention are prepared in the Examples section.

Compounds of formula (I) may be prepared as described in any of the Schemes below:

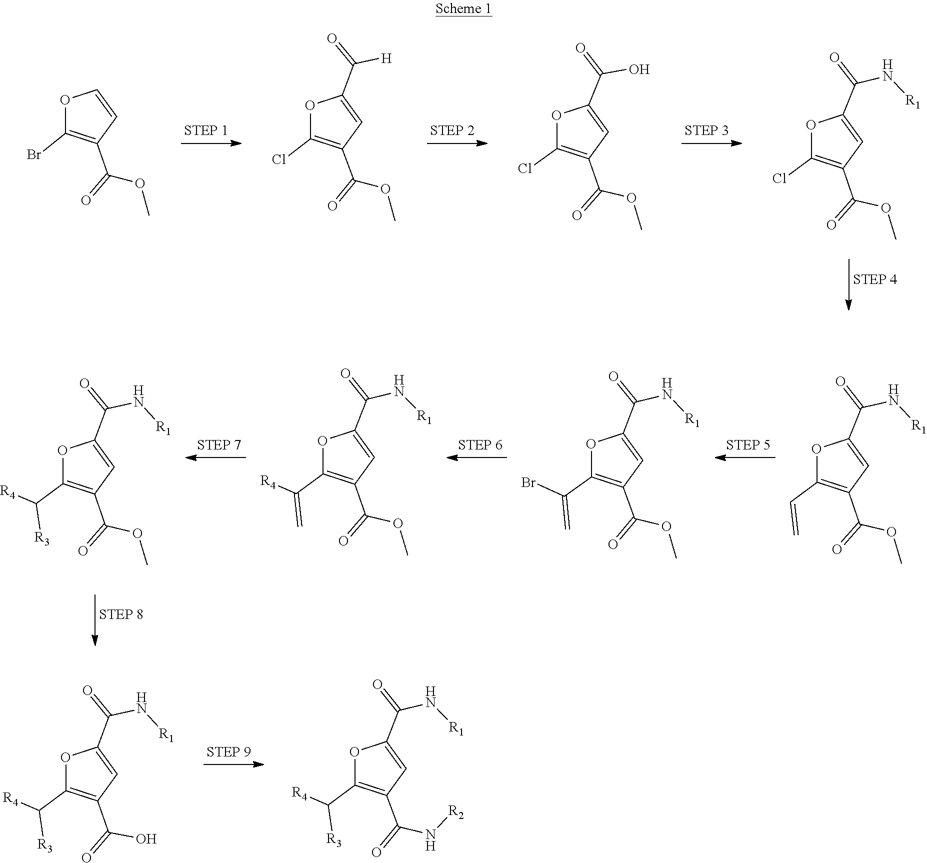

wherein R¹, R², R³ and R⁴ are as described above.

In respect of the steps shown in Scheme 1 above the following reaction conditions may be utilised:

Step 1: is a formylation reaction which may be carried out using standard formylation reagents such as POCl₃ in a suitable solvent such as DMF.
Step 2: is an oxidation reaction using suitable oxidising reagents such as sodium chlorite-hydrogen peroxide.
Step 3: is an amide coupling reaction and may be carried out using an amine reagent, R¹—NH₂, in the presence of a suitable tertiary amine, such as triethylamine or DIPEA, in the presence of a suitable amide coupling reactant, such as HATU, in a suitable solvent, such as DCM or DMF, at a suitable temperature, such as room temperature.
Step 4: is a Suzuki type coupling reaction using a suitable reagent such as 2,4,6-trivinylcyclotriboroxane-pyridine complex with a suitable palladium catalyst such as palladium (II) acetate, a suitable base such as caesium carbonate, in a suitable solvent such as DMF.
Step 5: is a bromination reaction using a suitable bromination reagent such as bromine or NBS.
Step 6: is a cross-coupling reaction and may be carried out by reaction with an appropriate organometallic reagent such as a boronic acid derivative (R₄—B(OH)₂) or an organotin reagent (e.g. R₄—Sn(C₄H₉)₃), in the presence of a palladium catalyst, in a suitable solvent such as 1,4-dioxane.
Step 7: is a hydrogenation reaction which may be carried out under suitable conditions such as under a H₂ atmosphere in the presence of a suitable catalyst, such as palladium on carbon at a suitable temperature and pressure.
Step 8: is a base-mediated ester hydrolysis and may be carried out using any suitable base, such as lithium hydroxide, optionally in a suitable solvent or mixture of solvents, such as 1,4-dioxane and water, at a suitable temperature, such as room temperature.
Step 9: is an amide coupling reaction and may be carried out using an amine reagent, R²—NH₂, in the presence of a suitable tertiary amine, such as triethylamine or DIPEA, in the presence of a suitable amide coupling reactant, such as HATU, in a suitable solvent, such as DCM or DMF, at a suitable temperature, such as room temperature.

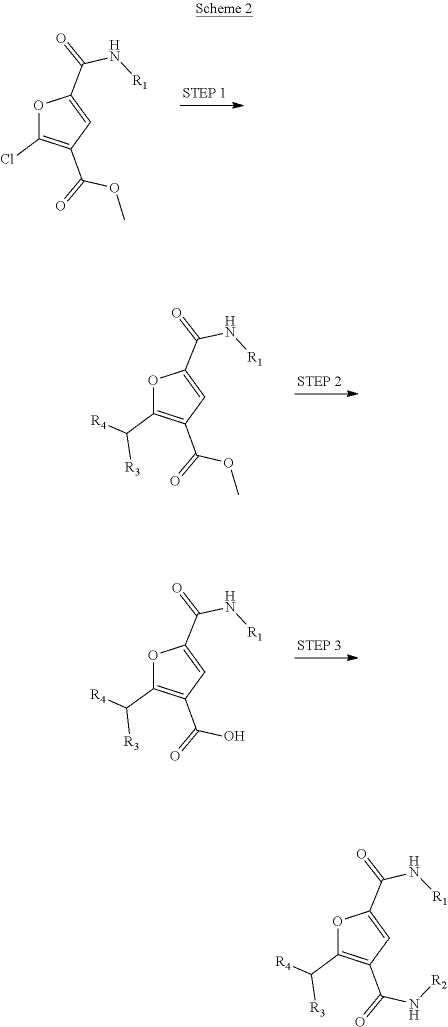

In respect of the steps shown in Scheme 2 above the following reaction conditions may be utilised:

Step 1: is a cross-coupling reaction and may be carried out by reaction with an appropriate organometallic reagent, such as an organozinc reagent (e.g. R₃R₄CH—Zn-Hal) in the presence of a suitable catalyst, such as bis(triphenylphosphine)palladium(II) dichloride, in a suitable solvent such as THF, at a suitable temperature, such as 90° C.
Step 2: is a base-mediated ester hydrolysis and may be carried out using a suitable base, such as lithium hydroxide, optionally in a suitable solvent or mixture of solvents, such as 1,4-dioxane and water, at a suitable temperature, such as room temperature.
Step 3: is an amide coupling reaction and may be carried out using an amine reagent, R²—NH₂, in the presence of a suitable tertiary amine, such as triethylamine or DIPEA, in the presence of a suitable amide coupling reactant, such as HATU, in a suitable solvent, such as DCM or DMF, at a suitable temperature, such as room temperature.

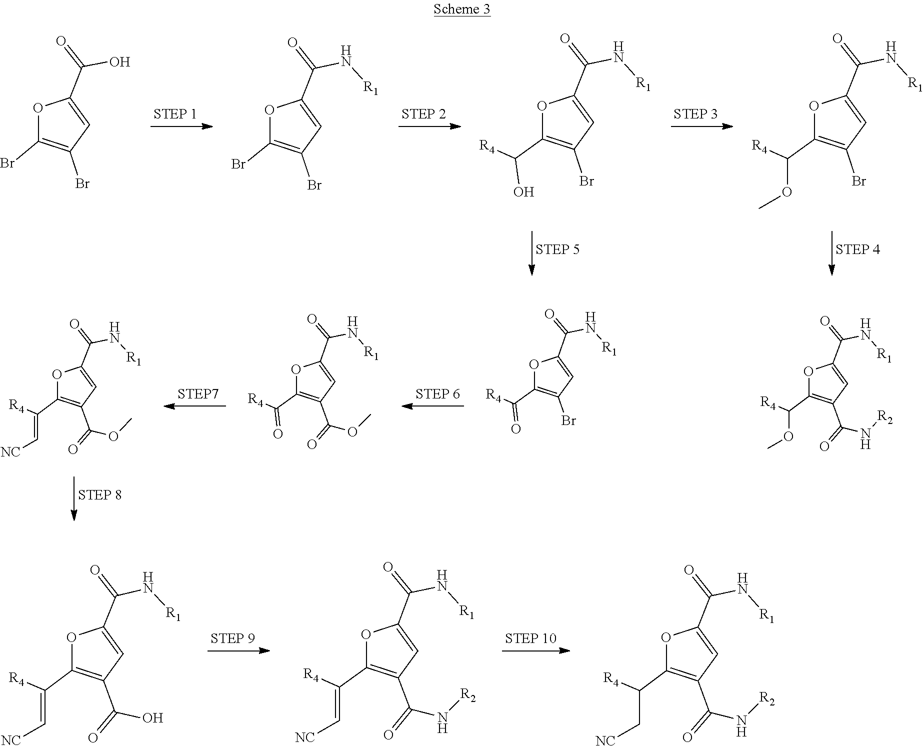

In respect of the steps shown in Scheme 3 above the following reaction conditions may be utilised:

Step 1: is an amide coupling reaction and may be carried out using an amine reagent, R¹—NH₂, in the presence of a suitable tertiary amine, such as triethylamine or DIPEA, in the presence of a suitable amide coupling reactant, such as HATU, in a suitable solvent, such as DCM or DMF, at a suitable temperature, such as room temperature.
Step 2: is a Grignard formation and subsequent addition to a suitable aldehyde (R₄C(O)H), using a suitable Grignard reagent, such as isopropyl magnesium bromide, in a suitable solvent, such as THF at a suitable temperature, such as −50° C. to 0° C.
Step 3: is a methylation reaction using an appropriate methylation reagent such as methyl iodide, optionally in the presence of a suitable additive, such as silver oxide, in a suitable solvent such as DMF, at a suitable temperature, such as 70° C.
Step 4: is an amidation reaction which may be carried out by reaction with a suitable amine (R²—NH₂), in the presence of a suitable source of CO, such as dicobalt octacarbonyl, in the presence of a suitable metal catalyst, such as a palladium catalyst, such as diacetoxypalladium, optionally in the presence of a suitable ligand, such as Xantphos, optionally in the presence of a suitable nucleophilic catalyst, such as DMAP, in a suitable solvent, such as 1,4-dioxane, at a suitable temperature, such as 90° C.
Step 5: is an oxidation reaction using a suitable oxidising agent, such as manganese dioxide, in a suitable solvent, such as dichloromethane, at a suitable temperature, such as room temperature.
Step 6: is a carbonylation reaction using a suitable palladium catalyst, such as palladium (II) acetate, optionally in the presence of a suitable ligand, such as Xantphos, optionally in the presence of a suitable base, such as triethylamine, in the presence of a suitable CO source, such as CO gas and a suitable solvent or solvent mixture such as DMF and methanol, at a suitable temperature, such as 70° C.
Step 7: is a Horner-Wadsworth-Emmons reaction and may be carried out using an appropriate phosphonate reagent such as diethyl (cyanomethyl)phosphonate, using a suitable base such as sodium hydride, in a suitable solvent such as THF, at a suitable temperature, such as 0° C. to room temperature.
Step 8: is a base-mediated ester hydrolysis and may be carried out using a suitable base, such as lithium hydroxide, optionally in a suitable solvent or mixture of solvents, such as THF and water, at a suitable temperature, such as room temperature.
Step 9: is an amide coupling reaction and may be carried out using an amine reagent, R²—NH₂, in the presence of a suitable tertiary amine, such as triethylamine or DIPEA, in the presence of a suitable amide coupling reactant, such as HATU, in a suitable solvent, such as DCM or DMF, at a suitable temperature, such as room temperature.
Step 10: is a reduction reaction which can be carried out using an appropriate reducing agent, such as sodium borohydride, in a suitable solvent such as isopropyl alcohol, at a suitable temperature, such as room temperature.

It will be appreciated by those skilled in the art that it may be advantageous to protect one or more functional groups of the compounds described above. Examples of protecting groups and the means for their removal can be found in T. W. Greene ‘Protective Groups in Organic Synthesis’ (4th edition, J. Wiley and Sons, 2006), incorporated herein by reference as it relates to such procedures.

Suitable amine protecting groups include acyl (e.g. acetyl, carbamate (e.g. 2′,2′,2′-trichloroethoxycarbonyl, benzyloxycarbonyl or t-butoxycarbonyl) and arylalkyl (e.g. benzyl), which may be removed by acid mediated cleavage (e.g. using an acid such as hydrochloric acid in 1,4-dioxane or trifluoroacetic acid in dichloromethane) or reductively (e.g. hydrogenolysis of a benzyl or benzyloxycarbonyl group or reductive removal of a 2′,2′,2′-trichloroethoxycarbonyl group using zinc in acetic acid) as appropriate. Other suitable amine protecting groups include trifluoroacetyl (—C(O)CF₃) which may be removed by base catalysed hydrolysis.

It will be appreciated that in any of the routes described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly.

Certain intermediate compounds described above form a yet further aspect of the invention.

For any of the hereinbefore described reactions or processes, conventional methods of heating and cooling may be employed, for example temperature-regulated oil-baths or temperature-regulated hot-blocks, and ice/salt baths or dry ice/acetone baths respectively. Conventional methods of isolation, for example extraction from or into aqueous or non-aqueous solvents may be used. Conventional methods of drying organic solvents, solutions, or extracts, such as shaking with anhydrous magnesium sulfate, or anhydrous sodium sulfate, or passing through a hydrophobic frit, may be employed. Conventional methods of purification, for example crystallisation and chromatography, for example silica chromatography or reverse-phase chromatography, may be used as required. Crystallisation may be performed using conventional solvents such as ethyl acetate, methanol, ethanol, or butanol, or aqueous mixtures thereof. It will be appreciated that specific reaction times and temperatures may typically be determined by reaction-monitoring techniques, for example thin-layer chromatography and LC-MS.

### General Experimental Details

All temperatures referred to are in ° C.

As used herein the symbols and conventions used in these processes, schemes and examples are consistent with those used in the contemporary scientific literature, for example, the Journal of the American Chemical Society. Unless otherwise noted, all starting materials were obtained from commercial suppliers and used without further purification. Specifically, the following abbreviations may be used in the examples and throughout the specification:

### General Methods

### General Experimental Details

As used herein the symbols and conventions used in these processes, schemes and examples are consistent with those used in the contemporary scientific literature, for example, the Journal of the American Chemical Society. Unless otherwise noted, all starting materials were obtained from commercial suppliers and used without further purification. Specifically, the following abbreviations may be used in the examples and throughout the specification:

### Abbreviations

AcOH acetic acid
AMU atomic mass units
Aq aqueous
BOC/Boc tert-butyloxycarbonyl
Cs₂CO₃ cesium carbonate
CHCl₃ chloroform
CMBP (cyanomethylene)tributylphosphorane
CPME cyclopentyl methyl ether
CV column volume
DCM dichloromethane
DIAD diisopropyl azodicarboxylate
DIBAL-H diisobutylaluminium hydride
DIPEA diisopropylethylamine
DMAP 4-dimethylaminopyridine
DMF dimethylformamide
DMSO dimethylsulfoxide
DMSO-d₆ deuterated dimethylsulfoxide
DPPA diphenylphosphoryl azide
Et₃N triethylamine
EtOAc ethyl acetate
EtOH ethanol
h hour(s)
HATU O-(7-azabenzotriazol-1-yl)-N,N,N′,N′-tetramethyluronium hexafluorophosphate
HBr hydrogen bromide
HCl hydrochloric acid
HPLC high performance liquid chromatography
Isolera Biotage® Flash purification system
K₂CO₃ potassium carbonate
LiCl lithium chloride
LCMS liquid chromatography-mass spectrometry
LiOH lithium hydroxide
M molar (concentration)
MDAP mass directed autopreparative chromatography
MeCN acetonitrile
MeOH methanol
2-MeTHF 2-methyltetrahydrofuran
min minute(s)
MS mass spectrometry
Ms-Cl methanesulfonyl chloride
N normal (concentration)
N₂ nitrogen gas
NaBH₄ sodium borohydride
Na₂CO₃ sodium carbonate
NaH sodium hydride
NaHCO₃ sodium bicarbonate
NaOH sodium hydroxide
Na₂SO₄ sodium sulphate
NH₃ ammonia
NH₄Cl ammonium chloride
NUT nuclear protein in testis
obs obscured
Ph₃P triphenylphosphine
RBF round bottomed flask
Rt retention time
rt room temperature
sat saturated
SCX Isolute strong cation exchange sorbent SPE
SFC supercritical fluid chromatography
SiO₂ silicon dioxide
SNAP Biotage® (silica) flash chromatography cartridge
SP4 Biotage® Flash purification system
SPE solid phase extraction
T₃P propylphosphonic anhydride solution
TFA trifluoroacetic acid
THF tetrahydrofuran
TBDMS-Cl tert-butyldimethylsilyl chloride
TLC Thin layer chromatography
Ts tosyl
pTsCl tosyl chloride
UPLC ultra performance liquid chromatography
UV ultraviolet
XantPhos 1,1′-(9,9-dimethyl-9H-xanthene-4,5-diyl)bis[1,1-diphenylphosphine

The names of the following compounds have been obtained using the compound naming programme “ACD Name Pro 6.02” or using the naming functionality of ChemDraw Ultra 12.0.

### LCMS Methodology

### Formic Method

### LC Conditions

The UPLC analysis was conducted on an Acquity UPLC CSH C18 column (50 mm×2.1 mm, i.d. 1.7 μm packing diameter) at 40° C.

The solvents employed were:

A=0.1% v/v solution of formic acid in water

B=0.1% v/v solution of formic acid in acetonitrile

The gradient employed was:

| Time (min) | Flow rate (mL/min) | % A | % B |
|---|---|---|---|
| 0 | 1 | 97 | 3 |
| 1.5 | 1 | 5 | 95 |
| 1.9 | 1 | 5 | 95 |
| 2.0 | 1 | 97 | 3 |

The UV detection was a summed signal from wavelength of 210 nm to 350 nm.

### MS Conditions

| | |
|---|---|
| MS: | Waters ZQ |
| Ionisation mode: | Alternate-scan positive and negative electrospray |
| Scan range: | 100 to 1000 AMU |
| Scan time: | 0.27 sec |
| Inter scan delay: | 0.10 sec |

### High pH Method

### LC Conditions

The UPLC analysis was conducted on an Acquity UPLC CSH C18 column (50 mm×2.1 mm, i.d. 1.7 μm packing diameter) at 40° C.

The solvents employed were:

A=10 mM ammonium hydrogen carbonate in water adjusted to pH10 with ammonia solution

B=acetonitrile

The gradient employed was:

| Time (min) | Flow rate (mL/min) | % A | % B |
|---|---|---|---|
| 0 | 1 | 97 | 3 |
| 0.05 | 1 | 97 | 3 |
| 1.5 | 1 | 5 | 95 |
| 1.9 | 1 | 5 | 95 |
| 2.0 | 1 | 97 | 3 |

The UV detection was a summed signal from wavelength of 210 nm to 350 nm.

### MS Conditions

| | |
|---|---|
| MS: | Waters ZQ |
| Ionisation mode: | Alternate-scan positive and negative electrospray |
| Scan range: | 100 to 1000 AMU |
| Scan time: | 0.27 sec |
| Inter scan delay: | 0.10 sec |

### TFA Method

### LC Conditions

The UPLC analysis was conducted on an Acquity UPLC CSH C18 column (50 mm×2.1 mm, i.d. 1.7 μm packing diameter) at 40° C.

The solvents employed were:

A=0.1% v/v solution of trifluoroacetic acid in water

B=0.1% v/v solution of trifluoroacetic acid in acetonitrile

The gradient employed was:

| Time (min) | Flow rate (mL/min) | % A | % B |
|---|---|---|---|
| 0 | 1 | 95 | 5 |
| 1.5 | 1 | 5 | 95 |
| 1.9 | 1 | 5 | 95 |
| 2.0 | 1 | 95 | 5 |

The UV detection was a summed signal from wavelength of 210 nm to 350 nm.

### MS Conditions

| | |
|---|---|
| MS: | Waters ZQ |
| Ionisation mode: | Alternate-scan positive and negative electrospray |
| Scan range: | 100 to 1000 AMU |
| Scan time: | 0.27 sec |
| Inter scan delay: | 0.10 sec |

### Method A

### LC Conditions

The UPLC analysis was conducted on an Acquity BEH C18 column (50 mm×2.1 mm, i.d. 1.7 μm packing diameter) at 35° C.

The solvents employed were:

A=0.1% v/v solution of formic acid in water

B=0.1% v/v solution of formic acid in acetonitrile

The gradient employed was:

| Time (min) | Flow rate (mL/min) | % A | % B |
|---|---|---|---|
| 0 | 0.6 | 97 | 3 |
| 0.4 | 0.6 | 97 | 3 |
| 3.2 | 0.6 | 2 | 98 |
| 3.8 | 0.6 | 2 | 98 |
| 4.2 | 0.6 | 97 | 3 |
| 4.5 | 0.6 | 97 | 3 |

### Method B

### LC Conditions

The UPLC analysis was conducted on an Acquity BEH C18 column (50 mm×2.1 mm, i.d. 1.7 μm packing diameter) at 35° C.

The solvents employed were:

A=0.050% v/v solution of formic acid in water B=0.050% v/v solution of formic acid in acetonitrile

The gradient employed was:

| Time (min) | Flow rate (mL/min) | % A | % B |
|---|---|---|---|
| 0 | 0.6 | 97 | 3 |
| 0.4 | 0.6 | 97 | 3 |
| 3.2 | 0.6 | 2 | 98 |
| 3.8 | 0.6 | 2 | 98 |
| 4.2 | 0.6 | 97 | 3 |
| 4.5 | 0.6 | 97 | 3 |

### Method C

### LC Conditions

The UPLC analysis was conducted on a Xbridge C18 column (150 mm×4.6 mm, i.d. 3.5 μm packing diameter) at 35° C.

The solvents employed were:

A=0.05% v/v solution of trifluoroacetic acid in water

B=acetonitrile

The gradient employed was:

| Time (min) | Flow rate (mL/min) | % A | % B |
|---|---|---|---|
| 0 | 1.0 | 95 | 5 |
| 0.5 | 1.0 | 95 | 5 |
| 7 | 1.0 | 5 | 95 |
| 14 | 1.0 | 5 | 95 |
| 14.5 | 1.0 | 95 | 5 |
| 15 | 1.0 | 95 | 5 |

### Method D

### LC Conditions

The UPLC analysis was conducted on an Acquity BEH C18 column (100 mm×2.1 mm, i.d. 1.7 μm packing diameter) at 35° C.

The solvents employed were:

A=0.050% v/v solution of formic acid in acetonitrile

B=0.050% v/v solution of formic acid in water

The gradient employed was:

| Time (min) | Flow rate (mL/min) | % A | % B |
|---|---|---|---|
| 0 | 0.45 | 3 | 97 |
| 0.4 | 0.45 | 3 | 97 |
| 7.5 | 0.45 | 98 | 2 |
| 9.5 | 0.45 | 98 | 2 |
| 9.6 | 0.45 | 3 | 97 |
| 10 | 0.45 | 3 | 97 |

### Method E

### LC Conditions

The UPLC analysis was conducted on an Acquity BEH C18 column (50 mm×2.1 mm, i.d. 1.7 μm packing diameter) at 35° C.

The solvents employed were:

A=0.05% v/v solution of formic acid in acetonitrile

B=0.05% v/v solution of formic acid in water

The gradient employed was:

| Time (min) | Flow rate (mL/min) | % A | % B |
|---|---|---|---|
| 0 | 0.45 | 3 | 97 |
| 0.4 | 0.45 | 3 | 97 |
| 4.0 | 0.45 | 98 | 2 |
| 4.5 | 0.45 | 97.5 | 2.5 |
| 5.0 | 0.45 | 3 | 97 |
| 5.5 | 0.45 | 3 | 97 |

General MDAP Purification Methods

Listed below are examples of mass-directed autopreparative chromatography (MDAP) methods that have been used or may be used in compound purification.

MDAP (High pH). The HPLC analysis was conducted on an Xselect CSH C18 column (150 mm×30 mm i.d. 5 μm packing diameter) at ambient temperature, eluting with 10 mM ammonium bicarbonate in water adjusted to pH 10 with ammonia solution (Solvent A) and acetonitrile (Solvent B) using an elution gradient of between 0 and 100% Solvent B over 15 or 25 min.

The UV detection was an averaged signal from wavelength of 210 nm to 350 nm. The mass spectra were recorded on a Waters ZQ Mass Spectrometer using alternate-scan positive and negative electrospray. Ionisation data was rounded to the nearest integer.

MDAP (Formic). The HPLC analysis was conducted on an Xselect CSH C18 column (150 mm×30 mm i.d. 5 μm packing diameter) at ambient temperature, eluting with 0.1% formic acid in water (Solvent A) and 0.1% formic acid in acetonitrile (Solvent B) using an elution gradient of between 0 and 100% solvent B over 15 or 25 min.

The UV detection was an averaged signal from wavelength of 210 nm to 350 nm. The mass spectra were recorded on a Waters ZQ Mass Spectrometer using alternate-scan positive and negative electrospray. Ionisation data was rounded to the nearest integer.

MDAP (TFA). The HPLC analysis was conducted on an Xselect CSH C18 column (150 mm×30 mm i.d. 5 μm packing diameter) at ambient temperature, eluting with 0.1% v/v solution of trifluoroacetic acid in water (Solvent A) and 0.1% v/v solution of trifluoroacetic acid in acetonitrile (Solvent B) using an elution gradient of between 0 and 100% solvent B over 15 or 25 min.

The UV detection was an averaged signal from wavelength of 210 nm to 350 nm. The mass spectra were recorded on a Waters ZQ Mass Spectrometer using alternate-scan positive and negative electrospray. Ionisation data was rounded to the nearest integer.

NMR

Spectra were run on either a 400 MHz or 600 MHz NMR machine at 302 K.

### Intermediate 1: 4,5-dibromo-N-methylfuran-2-carboxamide

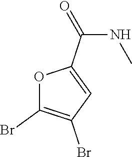

4,5-Dibromofuran-2-carboxylic acid (Commercially available from e.g. Sigma Aldrich, 1 g, 3.71 mmol) was and HATU (1.55 g, 4.08 mmol) were added to a flask, after which ethyl acetate (10.6 mL) was added and the solution stirred at rt for 1 h, after which time 2M methanamine in THF (2.04 mL, 4.08 mmol) was added and the reaction stirred at RT for 2.5 h. The reaction was diluted with ethyl acetate (40 mL) and washed with sat. aq. NaHCO₃ (50 mL). The aqueous layer was extracted with ethyl acetate (50 mL). The combined organics were washed with 2M aq. HCl (75 mL) and the acidic aqueous layer extracted with ethyl acetate (100 mL). The combined organics were passed through a hydrophobic frit and concentrated in vacuo. The residue was dissolved in DCM and loaded onto a pre-equilibrated 50 g Si column (40% EtOAc/Cyclohexane) and eluted on a gradient of 40-100% EtOAc/Cyclohexane over 12 column volumes. The appropriate fractions were combined and concentrated, and the solid dried in a vacuum oven to give the title compound (940 mg, 3.32 mmol, 90% yield) as a free flowing white solid. LCMS (2 min Formic): Rt=0.78 min, [MH]⁺=282.0/284.0/286.0

### Intermediate 2: Methyl 2-chloro-5-formylfuran-3-carboxylate

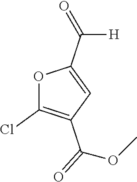

Methyl 2-bromofuran-3-carboxylate (Commercially available from e.g. Enamine, 4.96 g, 24.19 mmol) was taken up in DMF (3.75 mL, 48.4 mmol), cooled to 0° C. and put under nitrogen. POCl₃ (2.4 mL, 25.7 mmol) was added dropwise over 30 min, and the temperature was gradually increased to 80° C. over 1 hour. The reaction was left to stir for 20 hours before being allowed to cool to room temperature. The resulting brown tar was taken up in DMF (10 mL), and carefully poured into rapidly stirring ice-water. This was extracted with Et₂O (4×50 mL), and the combined organics were washed with saturated sodium bicarbonate solution (100 mL), dried with Na₂SO₄, filtered and concentrated in vacuo to yield the title compound (3.775 g, 17.02 mmol, 70.3% yield) as a pale orange solid.

¹H NMR (400 MHz, DMSO-d₆) δ ppm 9.57 (s, 1H) 7.89 (s, 1H) 3.85 (s, 3H).

### Intermediate 3: 5-Chloro-4-(methoxycarbonyl)furan-2-carboxylic Acid

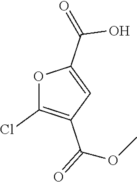

Methyl 2-chloro-5-formylfuran-3-carboxylate (For a preparation see Intermediate 2, 3.775 g, 20.02 mmol) was taken up in acetonitrile (36 mL) and water (36 mL). Sodium phosphate monobasic (13.21 g, 110 mmol) was added, followed by hydrogen peroxide (30% w/w in water, 11.25 mL, 110 mmol), and the reaction was cooled to 0° C. Sodium chlorite (7.22 g, 63.9 mmol) in water (36.0 mL) was added dropwise over 30 min, and the reaction allowed to warm to room temperature and stirred for 1 hour. 2 M HCl (10 mL) was added, and the reaction mixture was extracted with ethyl acetate (3×50 mL). The combined organics were dried with Na₂SO₄, filtered and concentrated in vacuo to yield the title compound (3.66 g, 16.1 mmol, 80% yield) as a cream solid.

LCMS (2 min Formic): Rt=0.78 min, [MH]⁺=203.2

### Intermediate 4: Methyl 2-chloro-5-(methylcarbamoyl)furan-3-carboxylate

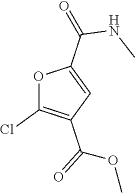

5-Chloro-4-(methoxycarbonyl)furan-2-carboxylic acid (For a preparation see Intermediate 3, 2.37 g, 11.6 mmol) was taken up in THF (50 mL). Triethylamine (4.85 mL, 34.8 mmol) was added, then 2 M methylamine in THF (17 mL, 34.0 mmol), and the reaction was cooled to 0° C. T3P® (50% w/w in ethyl acetate, 12 mL, 20.16 mmol) was added slowly, and the reaction left to warm to room temperature and stir for 2 hours during which time a precipitate formed. The mixture was cooled to 0° C. and filtered (washing with ice-cold THF) to isolate the title compound (1.12 g, 4.65 mmol, 40.1% yield) as a white solid. LCMS (2 min Formic): Rt=0.64 min, [MH]⁺=218.3.

### Intermediate 5: Methyl 5-(methylcarbamoyl)-2-vinylfuran-3-carboxylate

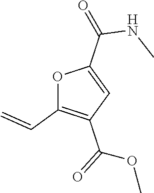

Methyl 2-chloro-5-(methylcarbamoyl)furan-3-carboxylate (For a preparation see Intermediate 4, 200 mg, 0.919 mmol), 2,4,6-Trivinylcyclotriboroxane pyridine complex (332 mg, 1.38 mmol), caesium carbonate (898 mg, 2.76 mmol), palladium (II) acetate (24 mg, 0.107 mmol) and cataCXium A (39 mg, 0.109 mmol) were combined in a 5 mL microwave vial in THF (2 mL) and water (1 mL). The resulting suspension was de-gassed and heated at 100° C. for 40 mins. The reaction mixture was cooled to rt, filtered through celite and concentrated to give approximately 364 mg crude orange solid. This was purified by chromatography on a silica gel cartridge, eluting with 0-100% ethyl acetate/cyclohexane. The appropriate fractions were combined and evaporated to give the title compound (113 mg, 0.486 mmol, 52.9% yield) as a pale yellow solid.

LCMS (2 min Formic): Rt=0.72 min, [MH]⁺=210.2.

### Intermediate 6: Methyl 2-(1-bromovinyl)-5-(methylcarbamoyl)furan-3-carboxylate

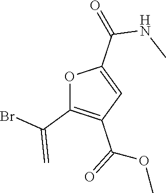 [SMILES: C=C(Br)c1oc(C(=O)NC)cc1C(=O)OC]

Methyl 5-(methylcarbamoyl)-2-vinylfuran-3-carboxylate (For a preparation see Intermediate 5, 107 mg, 0.511 mmol) was dissolved in dry DCM (3 mL). Bromine (0.04 mL, 0.767 mmol) was added and reaction mixture was stirred at rt under a nitrogen atmosphere. The reaction mixture was concentrated in vacuo to give an orange gum. Potassium hydroxide (52 mg, 0.927 mmol) was dissolved in methanol (3 mL) and warmed in a water bath at 45° C. This solution was then added to the orange gum and the reaction mixture stirred at rt for 15 mins. The reaction mixture was partitioned between water (10 mL), brine (5 mL) and ethyl acetate (10 mL). The organic layer was separated and the aqueous layer was extracted with further portions of ethyl acetate. The combined organic layers were dried by passing through a hydrophobic frit and concentrated to give the title compound (128 mg, 0.444 mmol, 78% yield) as a pale yellow solid. LCMS (2 min Formic): Rt=0.72 min, [MH]⁺=288.1/290.1.

### Intermediate 7: Methyl 2-(1-phenylethyl)furan-3-carboxylate

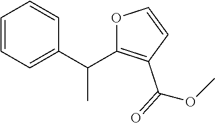 [SMILES: COC(=O)c1ccoc1C(C)c1ccccc1]

Methyl 2-bromofuran-3-carboxylate (Commercially available from e.g. Enamine, 1.5 g, 7.32 mmol) and bis(triphenylphosphine)palladium(II) chloride (0.514 g, 0.732 mmol) were put under a nitrogen atmosphere, and THF (20 mL) was added. (1-Phenylethyl)zinc(II) bromide (30 mL, 15.0 mmol) was added dropwise, and the reaction was left to stir at room temperature for 45 min. The reaction was quenched with saturated aqueous ammonium chloride (10 mL). The reaction mixture was diluted with ethyl acetate (50 mL), and water (50 mL) was added. This was filtered through Celite, and the filtrate was washed with brine (50 mL), dried with Na₂SO₄, filtered and concentrated in vacuo to yield a brown oil. The crude product was applied to a 50 g silica cartridge in the minimum of DCM and eluted with 0% ethyl acetate in cyclohexane for 2 column volumes then 0-7.5% ethyl acetate over 10 column volumes before being held at 7.5% for 5 column volumes. The appropriate fractions were combined and concentrated in vacuo to yield the title compound (843 mg, 3.11 mmol, 42.5% yield) as a yellow liquid. LCMS (2 min Formic): Rt=1.25 min, [MH]⁺=231.3.

### Intermediate 8: (+/−) Methyl 5-bromo-2-(1-phenylethyl)furan-3-carboxylate

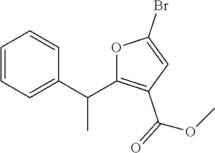 [SMILES: COC(=O)c1cc(Br)oc1C(C)c1ccccc1]

Methyl 2-(1-phenylethyl)furan-3-carboxylate (For a preparation see Intermediate 7, 843 mg, 3.66 mmol) was taken up in DMF (8 mL), cooled to 0° C. and put under a nitrogen atmosphere. N-Bromosuccinimide (658 mg, 3.70 mmol) was added in small portions over 15 min, and the reaction was allowed to warm to room temperature and stir for 2 hours. The reaction was quenched with water (10 mL), and ethyl acetate (20 mL) was added. An emulsion formed, so saturated LiCl solution (20 mL) was added, and the layers were separated. The organic layer was washed with water (2×15 mL), brine (20 mL), passed through a hydrophobic frit and concentrated in vacuo to yield the title compound (1.008 g, 3.10 mmol, 85% yield) as a yellow oil.

¹H NMR (400 MHz, DMSO-d₆) δ ppm 1.58 (d, J=7.34 Hz, 3H) 3.78 (s, 3H) 4.96 (q, J=7.34 Hz, 1H) 6.80 (s, 1H) 7.21-7.26 (m, 1H) 7.27-7.36 (m, 4H)

### Intermediate 9: (S)-Methyl 5-(methylcarbamoyl)-2-(1-phenylethyl)furan-3-carboxylate

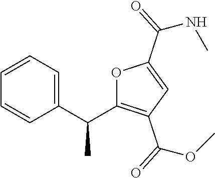 [SMILES: CNC(=O)c1cc(C(=O)OC)c([C@@H](C)c2ccccc2)o1]

Methanamine hydrochloride (275 mg, 4.08 mmol), palladium(II) acetate (73.2 mg, 0.326 mmol), xantphos (189 mg, 0.326 mmol), DMAP (896 mg, 7.34 mmol), imidazole (222 mg, 3.26 mmol) and dicobalt octacarbonyl (279 mg, 0.815 mmol) were sealed in a microwave vial and purged with nitrogen. Methyl 5-bromo-2-(1-phenylethyl)furan-3-carboxylate (For a preparation see Intermediate 8, 504 mg, 1.630 mmol) in 1,4-dioxane (12 mL) was added, and the reaction vessel was heated in a microwave reactor to 90° C. for 30 min. A second batch of reaction mixture was prepared in a separate microwave vial in an identical manner to that described above, and was also heated to 90° C. for 30 min. The two reaction mixtures were combined, diluted with ethyl acetate (50 mL) and water (50 mL), and filtered through Celite. The filtrate was separated and brine (50 mL) was added to the organic. This was filtered through Celite again to remove a grey precipitate, and the filtrate was separated. The organic layer was dried with MgSO₄, filtered and concentrated in vacuo to yield a brown solid. The crude product was applied to a 100 g silica cartridge in the minimum of DCM and eluted with 5% ethyl Acetate in cyclohexane for 2 column volumes, then 5-50% ethyl acetate over column volumes, then held at 50% for 5 column volumes. The appropriate fractions were combined and concentrated in vacuo to yield the racemic product methyl 5-(methylcarbamoyl)-2-(1-phenylethyl)furan-3-carboxylate (529 mg, 1.749 mmol, 54% yield) as a yellow gum.

The product was purified by chiral chromatography using the following conditions:

Column: 30 mm×25 cm Chiralpak IC

Flowrate: 30 mL/min

Solvents: 10% EtOH (+0.2% isopropylamine)/heptane (+0.2% isopropylamine)

The first eluting isomer was collected and the solvent removed by evaporation to give the title compound (213 mg, 0.704 mmol, 43.2% yield)

LCMS (2 min Formic): Rt=0.98 min, [MH]⁺=288.4.

### Intermediate 10: (S)-5-(Methylcarbamoyl)-2-(1-phenylethyl)furan-3-carboxylic Acid

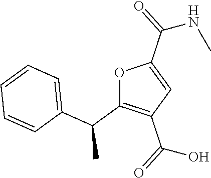 [SMILES: CNC(=O)c1cc(C(=O)O)c([C@@H](C)c2ccccc2)o1]

Methyl (S)-5-(methylcarbamoyl)-2-(1-phenylethyl)furan-3-carboxylate (For a preparation see Intermediate 9, 213 mg, 0.741 mmol) was taken up in 1,4-dioxane (4.5 mL) and water (3 mL). 1 M LiOH in water (1.48 mL, 1.48 mmol) was added, and the reaction left to stir at room temperature overnight. The reaction was concentrated in vacuo, the residue taken up in water (5 mL), acidified with 2M HCl (aq.), and the precipitate isolated by filtration and dried in a vacuum oven to yield the title compound (170 mg, 0.591 mmol, 80% yield) as a white solid

LCMS (2 min Formic): Rt=0.83 min, [MH]⁺=274.4.

### Intermediate 11: tert-Butyl(cyclopent-3-en-1-yloxy)dimethylsilane

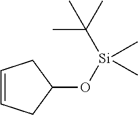

Cyclopent-3-en-1-ol (5 g, 59.4 mmol, commercially available from, for example, Astatech) was dissolved in DCM (100 mL) and TBDMS-Cl (8.96 g, 59.4 mmol) and imidazole (4.86 g, 71.3 mmol) were added, then the resulting suspension was stirred at r.t. over the weekend. The mixture was washed with water (2×100 mL), dried and evaporated in vacuo to give tert-butyl(cyclopent-3-en-1-yloxy)dimethylsilane (12.05 g, 60.7 mmol, 102% yield) as a pale yellow liquid. The product used crude without further purification.

¹H NMR (400 MHz, CDCl₃) δ ppm 5.68 (s, 2H) 4.50-4.62 (m, 1H) 2.59 (dd, J=14.9, 6.8 Hz, 2H) 2.23-2.37 (m, 2H) 0.91 (s, 9H) 0.09 (s, 6H).

### Intermediate 12: (1R,5S,6r)-Ethyl 3-((tert-butyldimethylsilyl)oxy)bicyclo[3.1.0]hexane-6-carboxylate, Mixture of Diastereomers

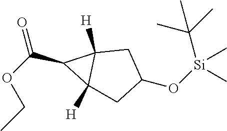 [SMILES: CCOC(=O)[C@H]1[C@@H]2CC(O[Si](C)(C)C(C)(C)C)C[C@@H]21]

Ethyl diazoacetate (6.90 mL, 66.5 mmol) was dissolved in DCM (150 mL) and added dropwise over ˜5 h to a mixture of rhodium (II) acetate dimer (1 g, 2.263 mmol) and tert-butyl(cyclopent-3-en-1-yloxy)dimethylsilane (For a preparation see Intermediate 11, 12 g, 60.5 mmol) in DCM (150 mL) at r.t. The resulting green solution was stirred overnight, then evaporated in vacuo to give a green liquid. This was loaded onto a 340 g silica column and eluted with 0-40% EtOAc/cyclohexane. The appropriate fractions were evaporated in vacuo to give the title compound (5.5 g, 19.3 mmol, 32.0% yield) as a colourless liquid, as a mixture of isomers at the silyl ether position (˜3:1 ratio) and this was carried through crude to the next step. LCMS (2 min High pH): Rt=0.96 min, [MH]⁺=not present.

### Intermediate 13: (1R,5S,6r)-3-((tert-butyldimethylsilyl)oxy)bicyclo[3.1.0]hexane-6-carboxylic Acid, Mixture of Diastereomers

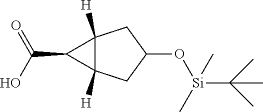 [SMILES: CC(C)(C)[Si](C)(C)OC1C[C@@H]2[C@H](C1)[C@H]2C(=O)O]

Sodium hydroxide (20 mL, 40.0 mmol, 2M aqueous solution) was added to a solution of ethyl (1R,5S,6r)-3-((tert-butyldimethylsilyl)oxy)bicyclo[3.1.0]hexane-6-carboxylate (For a preparation see Intermediate 12, 5.0 g, 17.6 mmol) in ethanol (50 mL) at rt and the mixture was stirred for 3 h. TLC suggested that all the starting material had been consumed and so the mixture was evaporated in vacuo to about 30 mL volume, then diluted with water (30 mL) and washed with ether (50 mL). The ether washings from the workup were dried and evaporated in vacuo to give recovered starting material: ethyl (1R,5S,6r)-3-((tert-butyldimethylsilyl)oxy)bicyclo[3.1.0]hexane-6-carboxylate (3.85 g). This was dissolved in ethanol (30 mL) and 2M aqueous NaOH solution (20 mL) was added, then the mixture was heated at 70° C. for 3 h, then evaporated in vacuo. The residue was dissolved in water (50 mL) and washed with ether (50 mL), then the aqueous layer was acidified with 2M HCl (20 mL) and extracted with EtOAc (2×50 mL). The combined organics were dried and evaporated in vacuo to give the title compound (1.9 g, 7.41 mmol, 42.2% yield) as a pale yellow solid, the NMR is consistent with a mixture of isomers. The product was carried through to the next step without purification.

### Intermediate 14: Benzyl ((1R,5S,6r)-3-((tert-butyldimethylsilyl)oxy)bicyclo[3.1.0]hexan-6-yl)carbamate, Mixture of Diastereomers

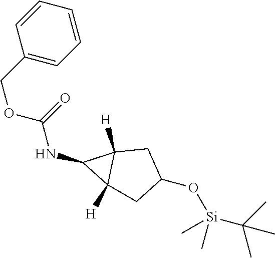 [SMILES: CC(C)(C)[Si](C)(C)OC1C[C@@H]2[C@H](C1)[C@H]2NC(=O)OCc1ccccc1]

(1R,5S,6r)-3-((tert-Butyldimethylsilyl)oxy)bicyclo[3.1.0]hexane-6-carboxylic acid (For a preparation see Intermediate 13, 1.8 g, 7.02 mmol) was dissolved in a mixture of toluene (20 mL) and Et₃N (1.96 mL, 14.04 mmol), then DPPA (1.815 mL, 8.42 mmol) was added and the mixture was stirred for 30 min at r.t. Benzyl alcohol (1.095 mL, 10.53 mmol) was added and the mixture heated at 100° C. for 4 h, then cooled to rt Ethyl acetate (100 mL) was added and the solution was washed with water (2×100 mL), then dried over sodium sulphate, filtered and the filtrate evaporated in vacuo to give a pale yellow oil. This was dissolved in DCM (10 mL) and loaded onto a 50 g silica column, then eluted with 0-30% EtOAc/cyclohexane and product-containing fractions (detected by permanganate dip) were collected and evaporated in vacuo to give the title compound (1.90 g, 5.26 mmol, 75% yield) as a pale yellow oil, the NMR is consistent with desired product as a mixture of isomers in approximately 2:1 ratio. The compound was taken through to the next step without further purification. LCMS (2 min Formic): Rt=1.56 min, [MH]⁺=362.6.

### Intermediate 15: (1R,5S,6r)-3-((tert-Butyldimethylsilyl)oxy)bicyclo[3.1.0]hexan-6-amine, Mixture of Diastereomers

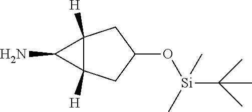

Benzyl ((1R,5S,6r)-3-((tert-butyldimethylsilyl)oxy)bicyclo[3.1.0]hexan-6-yl)carbamate (For a preparation see Intermediate 14, 1.9 g, 5.26 mmol) was dissolved in ethanol (100 mL) and hydrogenated in the H-Cube at atmospheric pressure and 1 mL/min flow rate. The eluent was evaporated in vacuo to give the title compound (1.12 g, 4.92 mmol, 84% yield) as a pale yellow oil. The product is an unequal mixture of diastereomers at the silyl ether position with a ratio of approximately 65:35.

¹H NMR (400 MHz, CDCl₃) δ ppm 4.22 ppm, 1H [A] (br.t, CH), 3.80 ppm 1H [B] (m, CH), 2.47 ppm, 1H [A] (t, CH), 2.05-1.93 ppm 2H [A]+3H [B] (m, 5×CH), 1.75-1.66 ppm —NH2 [A]+—NH2 [B]+2×CH [B}, 1.62 ppm 2H, [A] (dd, 2×CH). 1.20-1.15 ppm 2H [A]+2H [B] (M, 4×CH), 0.86 ppm, 9H [A] (s, 3×CH₃)+9H [B] (s, 3×CH₃), 0.00 ppm, 6H [A+B] (s, 2×CH₃)

### Intermediate 16: N⁴-((1R,5S,6r)-3-((tert-Butyldimethylsilyl)oxy)bicyclo[3.1.0]hexan-6-yl)-N²-methyl-5-((S)-1-phenylethyl)furan-2,4-dicarboxamide

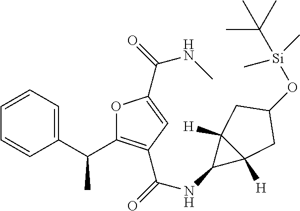 [SMILES: CNC(=O)c1cc(C(=O)N[C@H]2[C@@H]3CC(O[Si](C)(C)C(C)(C)C)C[C@@H]32)c([C@@H](C)c2ccccc2)o1]

(S)-5-(Methylcarbamoyl)-2-(1-phenylethyl)furan-3-carboxylic acid (For a preparation see Intermediate 10, 80 mg, 0.293 mmol) and (1R,5S,6r)-3-((tert-butyldimethylsilyl)oxy)bicyclo[3.1.0]hexan-6-amine (For a preparation see Intermediate 15, 96 mg, 0.422 mmol) were taken up in DMF (3 mL). DIPEA (0.153 mL, 0.878 mmol) was added, and the reaction left to stir for 10 min. HATU (167 mg, 0.439 mmol) was added, and the reaction left to stir at room temperature for 1.5 h. Toluene (10 mL) was added to the reaction to form an azeotrope with the DMF, and it was concentrated in vacuo. The residue was partitioned between ethyl acetate and sodium bicarbonate (10 mL of each). The organic layer was washed with brine (10 mL), dried with Na₂SO₄, filtered and concentrated in vacuo to yield a brown solid. The crude product was applied to a 10 g KP-Sil SNAP cartridge in the minimum of DCM and eluted with 5% ethyl acetate in cyclohexane for 2 column volumes then 5-50% ethyl acetate over 10 column volumes then held at 50% for 5 column volumes. The appropriate fractions were combined and concentrated in vacuo to yield the title compound (134 mg, 0.264 mmol, 90% yield) as a brown solid. LCMS (2 min Formic): Rt=1.50 min, [MH]⁺=483.3

### Intermediate 17: (+/−)-Methyl 2-(1-(3-chlorophenyl)ethyl)-5-(methylcarbamoyl)furan-3-carboxylate

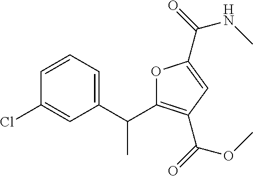 [SMILES: CNC(=O)c1cc(C(=O)OC)c(C(C)c2cccc(Cl)c2)o1]

(1-(3-Chlorophenyl)ethyl)zinc(II) bromide was prepared in the following manner: —Lithium chloride (232 mg, 5.47 mmol) was added to an oven dried 3 necked 50 ml r.b.f. under N₂. The flask was evacuated under vacuum and heated with a heat gun for 10 mins and backfilled with N₂. After cooling to rt zinc dust (357 mg, 5.47 mmol) was added. The flask was heated with a heat gun under vacuum for 10 minutes and backfilled with N₂. After cooling to rt THF (2 mL) was added followed by 1,2-dibromoethane (0.015 mL, 0.173 mmol) and the reaction mixture was heated at 60° C. for 20 mins. TMS-Cl (3 mg, 0.028 mmol) and iodine (4 mg, 0.016 mmol) in THF (0.5 mL) were added to the reaction mixture via syringe and the reaction mixture was heated for a further 20 mins at 60° C. 1-(1-Bromoethyl)-3-chlorobenzene (600 mg, 2.73 mmol, commercially available from, for example, Fluorochem) was added in THF (2 mL) and reaction mixture was stirred at 50° C. for 2 h. The product was assumed as a 0.6M solution, which was used as is for subsequent coupling. In a 5 mL microwave vial, methyl 2-chloro-5-(methylcarbamoyl)furan-3-carboxylate (for a preparation see Intermediate 4, 332 mg, 1.53 mmol) was suspended in dry THF (1 mL) and bis(triphenylphosphine)palladium(II) dichloride (321 mg, 0.458 mmol) was added. This was de-gassed and a solution of (1-(3-chlorophenyl)ethyl)zinc(II) bromide (4.5 mL, 2.70 mmol, 0.6M solution) was added via syringe. The reaction mixture was then heated at 90° C. for 1.5 h. Reaction mixture was filtered through Celite and concentrated to give a crude black residue. This was purified by silica gel column chromatography, eluting with 5-75% ethyl acetate/cyclohexane over 1250 mL. Fractions containing the desired product were combined and concentrated to give the title compound (253 mg, 0.668 mmol, 43.8% yield) as a yellow oil. LCMS (2 min Formic): Rt=1.07 min, [MH]⁺=322.2.

### Intermediate 18: (+/−)-2-(1-(3-Chlorophenyl)ethyl)-5-(methylcarbamoyl)furan-3-carboxylic Acid

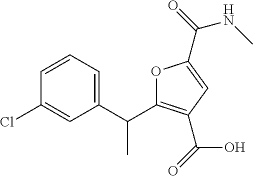 [SMILES: CNC(=O)c1cc(C(=O)O)c(C(C)c2cccc(Cl)c2)o1]

Methyl 2-(1-(3-chlorophenyl)ethyl)-5-(methylcarbamoyl)furan-3-carboxylate (For a preparation see Intermediate 17, 253 mg, 0.786 mmol) was dissolved in 1,4-dioxane (2 mL) and water (2 mL) was added, followed by LiOH (38 mg, 1.59 mmol). The Reaction mixture was stirred at rt for 2 h. The reaction mixture was then diluted with water and ethyl acetate and the aqueous layer was separated. The pH of the aqueous layer was adjusted with acetic acid (0.090 mL, 1.57 mmol) to approximately pH 4 and extracted with ethyl acetate (2×30 mL). The combined organic layers were dried (hydrophobic frit) and concentrated to give the title compound (242 mg, 0.788 mmol, 90% yield) as a colourless oil. LCMS (2 min Formic): Rt=0.94 min, [MH]⁺=308.2

### Intermediate 19: Methyl 5-(methylcarbamoyl)-2-(1-(6-methylpyridin-2-yl)vinyl)furan-3-carboxylate

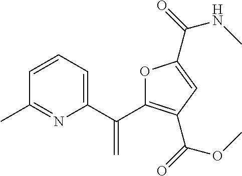

To a solution (part suspension that went into solution over time) of methyl 2-(1-bromovinyl)-5-(methylcarbamoyl)furan-3-carboxylate (For a preparation see Intermediate 6, 291 mg, 1.01 mmol) in 1,4-dioxane (3 mL) was added 2-methyl-6-(tributylstannyl)pyridine (0.340 mL, 1.01 mmol, commercially available from, for example, Sigma Aldrich) and the reaction mixture was de-gassed. Pd(Ph₃P)₄ (292 mg, 0.253 mmol) was added and the reaction mixture was stirred and heated in a microwave vial at 100° C. for 1.5 h. The reaction mixture was heated at 110° C. for a further 1 h. The crude reaction mixture was then combined with previous reactions using the same conditions on a smaller scale (with methyl 2-(1-bromovinyl)-5-(methylcarbamoyl)furan-3-carboxylate (50 mg, 0.174 mmol) and methyl 2-(1-bromovinyl)-5-(methylcarbamoyl)furan-3-carboxylate (50 mg, 0.174 mmol) and filtered through Celite. The reaction mixture was concentrated to give approximately 1.5 g of crude residue. This was purified by silica gel column chromatography, eluting with 0-100% ethyl acetate/cyclohexane over 480 mL). Fractions containing the desired product were combined and concentrated to give the title compound (130 mg, 0.433 mmol, 22.5%) as a yellow oil.

LCMS (2 min Formic): Rt=0.53 min, [MH]⁺=301.3.

### Intermediate 20: 5-(Methylcarbamoyl)-2-(1-(6-methylpyridin-2-yl)vinyl)furan-3-carboxylic Acid

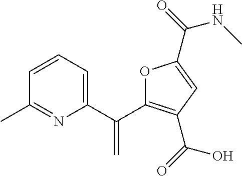

Methyl 5-(methylcarbamoyl)-2-(1-(6-methylpyridin-2-yl)vinyl)furan-3-carboxylate (for a preparation see Intermediate 19, 126 mg, 0.420 mmol) was dissolved in 1,4-dioxane (2 mL) and water (2 mL) and LiOH (21 mg, 0.877 mmol) was added. The reaction mixture was stirred at rt for 24 h. The reaction mixture was diluted with water and ethyl acetate and the aqueous layer was separated. The pH of the aqueous layer was adjusted with acetic acid (0.053 mL, 0.923 mmol) to approximately pH 4 and extracted with ethyl acetate (2×30 mL). The combined organic layers were dried by passing through a hydrophobic frit and concentrated to give the title compound (65 mg, 0.227 mmol, 46. % yield) as a pale yellow oil. LCMS (2 min Formic): Rt=0.40 min, [MH]⁺=287.2

### Intermediate 21: N⁴-((1r,4r)-4-Hydroxycyclohexyl)-N²-methyl-5-(1-(6-methylpyridin-2-yl)vinyl)furan-2,4-dicarboxamide

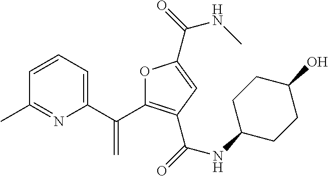 [SMILES: C=C(c1cccc(C)n1)c1oc(C(=O)NC)cc1C(=O)N[C@H]1CC[C@@H](O)CC1]

5-(Methylcarbamoyl)-2-(1-(6-methylpyridin-2-yl)vinyl)furan-3-carboxylic acid (for a preparation see Intermediate 20, 112 mg, 0.274 mmol) was dissolved in DMF (3 mL) and DIPEA (0.239 mL, 1.37 mmol) was added. (1r,4r)-4-Aminocyclohexan-1-ol (64 mg, 0.556 mmol,) was added followed by HATU (156 mg, 0.411 mmol) and the reaction mixture was stirred at r.t. for 5 h. The reaction mixture was combined with a previous reaction using the same conditions on a smaller scale (with 5-(methylcarbamoyl)-2-(1-(6-methylpyridin-2-yl)vinyl)furan-3-carboxylic acid (55 mg, 0.192 mmol) and partitioned between ethyl acetate and sat. aq. LiCl solution. The organic layer was separated and the aqueous layer further extracted with ethyl acetate (3×30 mL) as well as 25% isopropanol in CHCl₃ (3×50 mL). The combined organic layers were dried by passing through a hydrophobic frit and concentrated to give approximately 567 mg crude material. This was purified by silica gel column chromatography, eluting with 0-100% of 25% ethanol in ethyl acetate/cyclohexane over 330 mL) to give the title compound (123 mg, 0.321 mmol, 55.1% yield) as a yellow oil.

LCMS (2 min Formic): Rt=0.42 min, [MH]⁺=384.4

### Intermediate 22: Methyl 5-(methylcarbamoyl)-2-(1-(p-tolyl)vinyl)furan-3-carboxylate

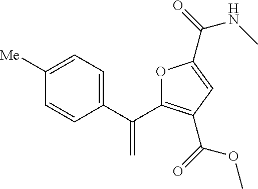 [SMILES: C=C(c1ccc(C)cc1)c1oc(C(=O)NC)cc1C(=O)OC]

Methyl 2-(1-bromovinyl)-5-(methylcarbamoyl)furan-3-carboxylate (For a preparation see Intermediate 6, 305 mg, 0.953 mmol) and p-tolylboronic acid (194 mg, 1.429 mmol) were combined in 1,4-dioxane (6 mL) and water (3 mL). Nitrogen was bubbled through the solution for 10 mins then potassium phosphate tribasic (708 mg, 3.33 mmol) and PEPPSI-IPr (129 mg, 0.191 mmol) were added. The reaction mixture was stirred at rt overnight. The reaction mixture was filtered through Celite and partitioned between ethyl acetate (20 mL) and water (20 mL). The organic layer was separated, dried (hydrophobic frit) and concentrated to give approximately 488 mg of crude orange residue. This was purified by silica gel column chromatography, eluting with 0-100% ethyl acetate/cyclohexane) to give the title compound (143 mg, 0.430 mmol, 45% yield) as a pale yellow oil.

LCMS (2 min Formic): Rt=1.03 min, [MH]⁺=300.2.

### Intermediate 23: (+/−)-Methyl 5-(methylcarbamoyl)-2-(1-(p-tolyl)ethyl)furan-3-carboxylate

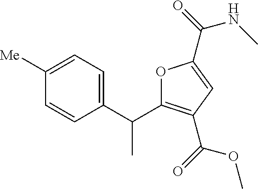 [SMILES: CNC(=O)c1cc(C(=O)OC)c(C(C)c2ccc(C)cc2)o1]

Methyl 5-(methylcarbamoyl)-2-(1-(p-tolyl)vinyl)furan-3-carboxylate (For a preparation see Intermediate 22, 146 mg, 0.488 mmol) was dissolved in a mixture of methanol (14 mL) and DCM (5 mL) and hydrogenated in a H-cube over a 10% Pd/C Catcart (51.9 mg, 0.488 mmol) at 1 mL/min flow rate, room temperature and atmospheric pressure for 1 pass. The reaction mixture was then concentrated to give the title compound (142 mg) as a colourless oil which was used without further purification in subsequent reactions. LCMS (2 min Formic): Rt=1.08 min, [MH]⁺=302.3.

### Intermediate 24: (+/−)-5-(Methylcarbamoyl)-2-(1-(p-tolyl)ethyl)furan-3-carboxylic Acid

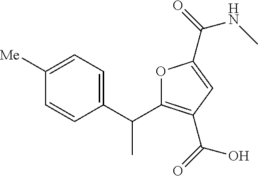 [SMILES: CNC(=O)c1cc(C(=O)O)c(C(C)c2ccc(C)cc2)o1]

(+/−)-Methyl 5-(methylcarbamoyl)-2-(1-(p-tolyl)ethyl)furan-3-carboxylate (For a preparation see Intermediate 23, 142.5 mg, 0.473 mmol) was dissolved in 1,4-dioxane (3 mL) and water (3 mL) was added, followed by LiOH (23.3 mg, 0.973 mmol). The reaction mixture was stirred at rt in air overnight. The reaction mixture was then diluted with water and ethyl acetate and the aqueous layer was separated. The pH of the aqueous layer was adjusted with acetic acid (0.054 mL, 0.946 mmol) to approximately pH 4 and extracted with ethyl acetate (2×30 mL). The combined organic layers were dried by passing through a hydrophobic frit and concentrated to give the title compound (133 mg, 0.463 mmol, 98% yield) as a colourless oil which was used without further purification in subsequent reactions. LCMS (2 min Formic): Rt=0.92 min, [MH]⁺=288.3.

### Intermediate 25: Methyl 2-(1-(3-chloro-4-methoxyphenyl)vinyl)-5-(methylcarbamoyl)furan-3-carboxylate

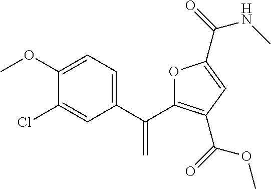

Methyl 2-(1-bromovinyl)-5-(methylcarbamoyl)furan-3-carboxylate (For a preparation see Intermediate 6, 413 mg, 1.29 mmol) and (3-chloro-4-methoxyphenyl)boronic acid (363 mg, 1.95 mmol) were combined in 1,4-dioxane (3 mL) and water (1.5 mL). Nitrogen was bubbled through the solution for 10 min then potassium phosphate tribasic (960 mg, 4.52 mmol) and PEPPSI-Ir (178 mg, 0.262 mmol) were added. The reaction mixture was stirred at rt with N₂ bubbled through for 5 h. The reaction mixture was filtered through Celite and partitioned between ethyl acetate (20 mL) and water (20 mL). The organic layer was separated, dried by passing through a hydrophobic frit and concentrated to give approximately 1 g of crude orange residue. This was purified by silica gel column chromatography, eluting with 0-100% ethyl acetate/cyclohexane to give the title compound (275 mg, 0.786 mmol, 58.4% yield) as a light yellow solid which was used without further purification in subsequent reactions. LCMS (2 min Formic): Rt=1.01 min, [MH]⁺=350.2.

### Intermediate 26: (+/−)-Methyl 2-(1-(3-chloro-4-methoxyphenyl)ethyl)-5-(methylcarbamoyl)furan-3-carboxylate

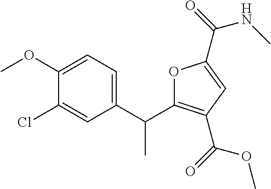

Methyl 2-(1-(3-chloro-4-methoxyphenyl)vinyl)-5-(methylcarbamoyl)furan-3-carboxylate (For a preparation see Intermediate 25, 275 mg, 0.786 mmol) was dissolved in a mixture of methanol (14 mL) and DCM (5 mL) and hydrogenated in a H-cube over a 10% Pd/C Catcart (84 mg, 0.786 mmol) at 1 mL/min flow rate, room temperature and atmospheric pressure for 1 pass. The reaction mixture was then concentrated to give the title compound (271 mg, 0.770 mmol, 83% yield) as a colourless oil which was used without further purification in subsequent reactions.

LCMS (2 min Formic): Rt=1.05 min, [MH]⁺=352.2.

### Intermediate 27: (+/−)-2-(1-(3-Chloro-4-methoxyphenyl)ethyl)-5-(methylcarbamoyl)furan-3-carboxylic Acid

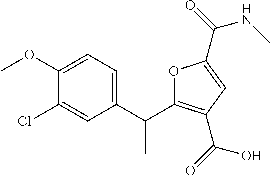

(+/−)-Methyl 2-(1-(3-chloro-4-methoxyphenyl)ethyl)-5-(methylcarbamoyl)furan-3-carboxylate (For a preparation see Intermediate 26, 271 mg, 0.770 mmol) was dissolved in 1,4-dioxane (3 mL) and water (3 mL) was added, followed by LiOH (38 mg, 1.587 mmol). The reaction mixture was stirred at r.t. overnight. The reaction mixture was then diluted with water and ethyl acetate and the aqueous layer was separated. The pH of the aqueous layer was adjusted with acetic acid (0.088 mL, 1.54 mmol) to approximately pH 4 and then extracted with ethyl acetate (2×30 mL). The combined organic layers were dried by passing through a hydrophobic frit and concentrated to give the title compound (133 mg, 0.393 mmol, 35.9% yield) as a colourless oil which was used without purification in subsequent reactions. LCMS (2 min Formic): Rt=0.92 min, [MH]⁺=338.2.

### Intermediate 28: 4-Bromo-5-(3-chlorobenzoyl)-N-methylfuran-2-carboxamide

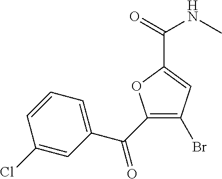 [SMILES: CNC(=O)c1cc(Br)c(C(=O)c2cccc(Cl)c2)o1]

To an oven-dried microwave vial was added 4,5-dibromo-N-methylfuran-2-carboxamide (For a preparation see Intermediate 1, 6 g, 21.2 mmol). The vial was sealed and purged with N₂. Anhydrous THF (60.6 mL) was added and the reaction cooled to −45° C. and isopropyl magnesium chloride 1.26 M in THF (33.8 mL, 42.6 mmol) was added dropwise and the reaction stirred at −50° C. for 30 min. 3-Chlorobenzaldehyde was added (9.61 mL, 85 mmol) and the reaction warmed to 0° C. over 1 h. The reaction was quenched with 0.5 M HCl (aq., 100 mL) and diluted with EtOAc (100 mL). The layers were shaken and separated, after which the aqueous was extracted with EtOAc (2×100 mL) and the combined organics passed through a hydrophobic frit and concentrated in vacuo. The residue was purified by silica gel chromatography (20-80% 3:1 EtOAc:EtOH/cyclohexane). The fractions corresponding to major eluting compound were combined and concentrated to give a yellow solid. This was dissolved in MeOH/DCM and purified by silica gel chromatography (0-5% MeOH/DCM). Manganese dioxide (22.12 g, 254 mmol) was added to the residue, followed by DCM (200 mL) and the reaction mixture stirred at rt for 40 min. The reaction was filtered through Celite and concentrated in vacuo to give the title compound as a white foam (3.7 g, 10.82 mmol, 51% yield).

LCMS (2 min Formic): Rt=1.05 min, [MH]⁺=342.0.

### Intermediate 29: Methyl 2-(1-(3-Chlorophenyl)-2-cyanovinyl)-5-(methylcarbamoyl)furan-3-carboxylate

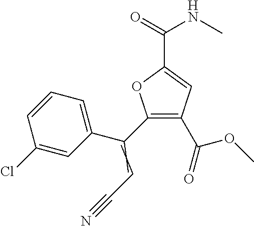 [SMILES: CNC(=O)c1cc(C(=O)OC)c(C(=CC#N)c2cccc(Cl)c2)o1]

Step 1: 4-Bromo-5-(3-chlorobenzoyl)-N-methylfuran-2-carboxamide (For a preparation see Intermediate 28, 100 mg, 0.234 mmol), triethylamine (0.065 mL, 0.467 mmol), palladium(II) acetate (5.2 mg, 0.023 mmol) and Xantphos (13.5 mg, 0.023 mmol) were added into two vials followed by DMF (0.7 mL) and methanol (0.350 mL). The vials were closed using a metal crimp cap and the septa were pierced with a needle. The reaction was stirred for 16 h at 70° C. under CO (5 bar). The contents of the first vial was purified by silica gel chromatography (0-100% EtOAc/Cyclohexane) to give the intermediate methyl 2-(3-chlorobenzoyl)-5-(methylcarbamoyl)furan-3-carboxylate as a yellow solid (37.4 mg, 0.107 mmol, 45.8% yield) which was used directly in Step 2.

Step 2: To a heat gun dried flask was added sodium hydride (60% in mineral oil, 68.6 mg, 1.72 mmol) and the vial sealed and purged with N₂. THF (3.81 mL) was added and the mixture cooled to 0° C. Diethyl (cyanomethyl)phosphonate (278 μL, 1.72 mmol) was added and the mixture stirred for 15 min. A solution of methyl 2-(3-chlorobenzoyl)-5-(methylcarbamoyl)furan-3-carboxylate (200 mg, 0.572 mmol) in THF (7.63 mL) was added and the reaction stirred at r.t. for 30 min. The reaction mixture was diluted with 2M aq HCl (4 mL) and extracted into EtOAc (50 mL). The organics were combined and passed through a hydrophobic frit and concentrated in vacuo. The residue was taken up into DCM and purified by silica gel chromatography eluting on a gradient of 20-60% 3:1 EtOAC:EtOH/cyclohexane. The fractions corresponding to the major eluting compound were combined and evaporated to give the crude product. This crude product was dissolved in DCM (10 mL) and washed with sat. aq. NaHCO₃ (5 mL). The organic layer was passed through a hydrophobic frit and concentrated under stream of nitrogen and dried in a heat piston to give a brown oil, which was purified by silica gel chromatography, eluting with a gradient of 20-60% EtOAc/Cyclohexane. The appropriate fractions were combined and concentrated in vacuo to give the title compound (146 mg, 0.423 mmol, 74.0% yield) as a white solid.

LCMS (2 min Formic): Rt=0.97/0.99 min, [MH]⁺=345.2

### Intermediate 30: 2-(1-(3-Chlorophenyl)-2-cyanovinyl)-5-(methylcarbamoyl)furan-3-carboxylic Acid

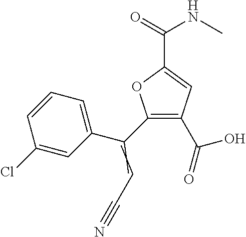 [SMILES: CNC(=O)c1cc(C(=O)O)c(C(=CC#N)c2cccc(Cl)c2)o1]

Methyl 2-(1-(3-chlorophenyl)-2-cyanovinyl)-5-(methylcarbamoyl)furan-3-carboxylate (For a preparation see Intermediate 29, 95 mg, 0.276 mmol) and lithium hydroxide (13.2 mg, 0.551 mmol) were dissolved in THF (459 μL) and water (459 μL) and the reaction stirred at rt for 2 h, after which the reaction was diluted with 2M HCl (aq., 2 mL) and extracted into EtOAc (2×10 mL). The organics were passed through a hydrophobic frit and concentrated in vacuo to give the title compound as a white solid. (90 mg, 0.272 mmol, 99% yield).

LCMS (2 min Formic): Rt=0.86, 0.89 min, [MH]⁺=331.1, suggestive of 40/60% mixture of stereoisomers.

### Intermediate 31: (+/−)-4-Bromo-5-((3-chlorophenyl)(hydroxy)methyl)-N-methylfuran-2-carboxamide

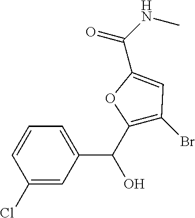 [SMILES: CNC(=O)c1cc(Br)c(C(O)c2cccc(Cl)c2)o1]

To an oven-dried microwave vial was added 4,5-dibromo-N-methylfuran-2-carboxamide (For a preparation see Intermediate 1, 4 g, 14.14 mmol). The vial was sealed and purged with N₂. Anhydrous tetrahydrofuran (40.4 mL) was added and the reaction cooled to −45° C. Isopropyl magnesium chloride (1.26 M in THF, 22.55 mL, 28.4 mmol) was added dropwise and the reaction stirred at −45° C. for 30 min. To the reaction was added 3-chlorobenzaldehyde (6.41 mL, 56.6 mmol) and the reaction stirred at between −45 and −20° C. for 1.5 h. The reaction was quenched with aq. HCl (0.5 M, 100 mL) and EtOAc (100 mL). The layers were shaken and separated, after which the aqueous was extracted with EtOAc (2×100 mL) and the combined organics passed through a hydrophobic frit and concentrated in vacuo. The residue was loaded onto silica (330 g, prewashed with 20% 3:1 EtOAc:EtOH/cyclohexane) eluting with a gradient of 20-80% 3:1 EtOAc:EtOH/cyclohexane. The relevant fractions were combined and concentrated to give the title compound (1.61 g, 4.67 mmol, 33% yield) as a yellow solid. LCMS (2 min Formic): Rt=0.94 min, [MH]⁺=344.1.

### Intermediate 32: (+/−)-4-Bromo-5-((3-chlorophenyl)(methoxy)methyl)-N-methylfuran-2-carboxamide

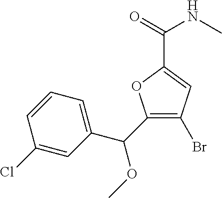 [SMILES: CNC(=O)c1cc(Br)c(C(OC)c2cccc(Cl)c2)o1]

(+/−) 4-Bromo-5-((3-chlorophenyl)(hydroxy)methyl)-N-methylfuran-2-carboxamide (For a preparation see Intermediate 31, 500 mg, 1.45 mmol) and silver oxide (672 mg, 2.90 mmol) were added to a vial and dissolved in DMF (4837 μL). Methyl iodide (1361 μL, 21.8 mmol) was added and the reaction stirred at 70° C. for 21 h after which the reaction was filtered through Celite and concentrated in vacuo. This was purified by silica chromatography eluting on a gradient of 40-100% EtOAc/cyclohexane. Fractions corresponding to the major eluting peak were combined and concentrated to give the title compound (400 mg, 1.12 mmol, 77% yield) as a yellow solid.

LCMS (2 min Formic): Rt=1.12 min, [MH]⁺=360.2.

### Intermediate 33: Methyl 2-(1-(1H-indol-4-yl)vinyl)-5-(methylcarbamoyl)furan-3-carboxylate

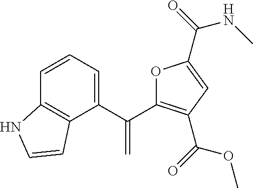 [SMILES: C=C(c1oc(C(=O)NC)cc1C(=O)OC)c1cccc2[nH]ccc12]

Methyl 2-(1-bromovinyl)-5-(methylcarbamoyl)furan-3-carboxylate (For a preparation see Intermediate 6, 900 mg, 3.12 mmol) and (1H-indol-4-yl)boronic acid (754 mg, 4.69 mmol, commercially available from, for example, Frontier Scientific) were dissolved in 1,4-Dioxane (20 mL) and water (10 mL). The mixture was purged with nitrogen-vacuum cycles and nitrogen was bubbled through the mixture for 5 mins. Then, PEPPSI-SIPr catalyst (426 mg, 0.625 mmol) and potassium phosphate tribasic (2.32 g, 10.9 mmol) were added to the mixture. This was stirred overnight at room temperature. The crude reaction was filtrated through a Celite cartridge, and then partitioned between ethyl acetate and water. The aqueous layer was extracted one more time with ethyl acetate. The organic layers were combined, dried over Na₂SO₄, filtered and concentrated in vacuo to give a black oil. The crude product was applied to a 50 g silica cartridge in the minimum of DCM and eluted with 0% ethyl acetate in cyclohexane for 1 column volume then 0-100% ethyl acetate over 12 column volumes then held at 100% for 3 column volumes. The appropriate fractions were combined and concentrated in vacuo to give the title compound (620 mg, 1.82 mmol, 58.1% yield) as a yellow solid.

LCMS (2 min Formic): Rt=0.89 min, [MH]⁺=325.3

### Intermediate 34: (+/−) Ethyl 2-(1-(1H-indol-4-yl)ethyl)-5-(methylcarbamoyl)furan-3-carboxylate

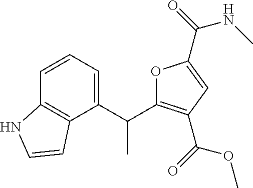 [SMILES: CNC(=O)c1cc(C(=O)OC)c(C(C)c2cccc3[nH]ccc23)o1]

Methyl 2-(1-(1H-indol-4-yl)vinyl)-5-(methylcarbamoyl)furan-3-carboxylate (For a preparation see Intermediate 33, 620 mg, 1.91 mmol) was taken up in ethanol (20 mL) with 5% wt. palladium on carbon (407 mg, 0.191 mmol) then added. The mixture was stirred at room temperature under hydrogen (3.85 mg, 1.91 mmol) for 6 h. The reaction mixture was filtrated through a Celite cartridge. The filtrate was concentrated in vacuo to give the title compound (587 mg, 1.71 mmol, 89% yield) as a brown solid. LCMS (2 min Formic): Rt=0.91 min, [MH]⁺=327.4

### Intermediate 35: (+/−) 2-(1-(1H-Indol-4-yl)ethyl)-5-(methylcarbamoyl)furan-3-carboxylic Acid

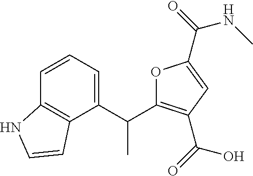 [SMILES: CNC(=O)c1cc(C(=O)O)c(C(C)c2cccc3[nH]ccc23)o1]

(+/−) Methyl 2-(1-(1H-indol-4-yl)ethyl)-5-(methylcarbamoyl)furan-3-carboxylate (For a preparation see Intermediate 34, 587 mg, 1.80 mmol) was taken up in methanol (10 mL), sodium hydroxide 2M aqueous solution (2.70 mL, 5.40 mmol) was added and the mixture was stirred for 8 h at room temperature. The reaction mixture was acidified with aqueous HCl 2M and extracted with ethyl acetate. The organic layers were combined, dried over Na₂SO₄, filtrated and concentrated in vacuo to give the title compound (583 mg, 1.57 mmol, 87% yield) as a brown-red solid.

LCMS (2 min Formic): Rt=0.78 min, [MH]⁺=313.3

### Intermediate 36: Methyl 2-(1-(1H-pyrrolo[2,3-b]pyridin-4-yl)vinyl)-5-(methylcarbamoyl)furan-3-carboxylate

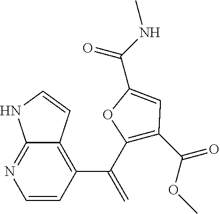 [SMILES: C=C(c1oc(C(=O)NC)cc1C(=O)OC)c1ccnc2[nH]ccc12]

Methyl 2-(1-bromovinyl)-5-(methylcarbamoyl)furan-3-carboxylate (for a preparation see Intermediate 6, 1.4 g, 4.86 mmol) and (1H-pyrrolo[2,3-b]pyridin-4-yl)boronic acid (0.866 g, 5.35 mmol) were dissolved in 1,4-dioxane (30 mL) and water (15 mL). The mixture was purged with nitrogen/vacuum cycles and nitrogen was bubbled through the mixture for 5 minutes. Then, PEPPSI-SIPr (0.662 g, 0.972 mmol) and potassium phosphate tribasic (3.61 g, 17.01 mmol) were added to the mixture. This was stirred over the weekend at room temperature. The crude was filtered through a celite cartridge, partitioned between ethyl acetate and Water. The layers were separated, and the aqueous layer was extracted one further time with ethyl acetate. The organic layers were combined, dried over Na₂SO₄, filtered and concentrated in vacuo to give an orange solid. The crude product was purified by silica gel column chromatography, eluting with 0% (3:1 EtOAc:EtOH) in cyclohexane for 2 column volumes, then 0-100% (3:1 EtOAc:EtOH) over 12 column volumes then held at 100% for 3 column volumes. The appropriate fractions were combined and concentrated in vacuo to give the desired product as a yellow solid (772 mg, 2.28 mmol, 46.9% yield).

LCMS (2 min Formic): Rt=0.61 min, [MH]⁺=326.2

### Intermediate 37: Methyl 2-(1-(1H-pyrrolo[2,3-b]pyridin-4-yl)ethyl)-5-(methylcarbamoyl)furan-3-carboxylate

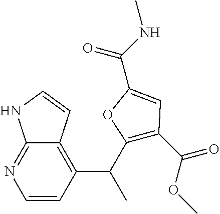 [SMILES: CNC(=O)c1cc(C(=O)OC)c(C(C)c2ccnc3[nH]ccc23)o1]

Methyl 2-(1-(1H-pyrrolo[2,3-b]pyridin-4-yl)vinyl)-5-(methylcarbamoyl)furan-3-carboxylate (for a preparation see Intermediate 36, 770 mg, 2.367 mmol) was dissolved in ethanol (20 mL), and 5% palladium on carbon (504 mg, 0.237 mmol) was added. The mixture was stirred at room temperature under an atmosphere of hydrogen (4.77 mg, 2.367 mmol) for 2 hours. The mixture was filtered through a celite cartridge. The filtrate was concentrated in vacuo to give the desired product (668 mg, 1.979 mmol, 84% yield) as a grey solid. LCMS (2 min Formic): Rt=0.60 min, [MH]⁺=328.2

### Intermediate 38: 2-(1-(1H-pyrrolo[2,3-b]pyridin-4-yl)ethyl)-5-(methylcarbamoyl)furan-3-carboxylic Acid

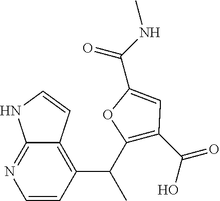 [SMILES: CNC(=O)c1cc(C(=O)O)c(C(C)c2ccnc3[nH]ccc23)o1]

Methyl 2-(1-(1H-pyrrolo[2,3-b]pyridin-4-yl)ethyl)-5-(methylcarbamoyl)furan-3-carboxylate (for a preparation see Intermediate 37, 668 mg, 2.04 mmol) was taken up in methanol (20 mL), 2M sodium hydroxide (3.06 mL, 6.12 mmol) was added and the mixture was stirred for 20 hours at room temperature. The reaction mixture was acidified with 3 mL of HCl 2M, and then concentrated in vacuo to give the desired product (1.08 g, 1.90 mmol, 93% yield) as a brown-yellow solid.

LCMS (2 min Formic): Rt=0.49 min, [MH]⁺=314.2

### EXAMPLES

### Example 1: N⁴-((1R,3R,5S,6r)-3-Hydroxybicyclo[3.1.0]hexan-6-yl)-N²-methyl-5-((S)-1-phenylethyl)furan-2,4-dicarboxamide

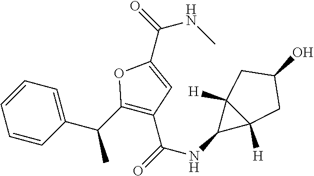 [SMILES: CNC(=O)c1cc(C(=O)N[C@H]2[C@@H]3C[C@@H](O)C[C@@H]32)c([C@@H](C)c2ccccc2)o1]

4 M HCl in dioxane (2 mL, 8.00 mmol) was added to N⁴-((1R,5S,6r)-3-((tert-butyldimethylsilyl)oxy)bicyclo[3.1.0]hexan-6-yl)-N²-methyl-5-((S)-1-phenylethyl)furan-2,4-dicarboxamide (For a preparation see Intermediate 16, 134 mg, 0.278 mmol) and sonicated for 5 min. A brown solid precipitated, and the reaction mixture was concentrated in vacuo. The crude product was purified by MDAP (Formic). The first eluting peak was concentrated in vacuo to yield the title compound (37 mg, 0.095 mmol, 34.4% yield) as a cream coloured solid.

LCMS (2 min Formic): Rt=0.78 min, [MH]⁺=369.2

¹H NMR (400 MHz, DMSO-d₆) δ ppm 8.30 (d, J=4.65 Hz, 1H) 8.00 (d, J=3.91 Hz, 1H) 7.39 (m, J=1.00 Hz, 3H) 7.25-7.34 (m, 2H) 7.15-7.24 (m, 1H) 5.16 (q, J=7.25 Hz, 1H) 4.57 (d, J=5.38 Hz, 1H) 3.74-3.88 (m, 1H) 2.76 (d, J=4.65 Hz, 3H) 2.40-2.45 (m, 1H) 2.02 (ddd, J=12.41, 7.03, 4.03 Hz, 2H) 1.54-1.64 (m, 5H) 1.34-1.42 (m, 2H)

### Example 2: (+/−) 5-(1-(3-Chlorophenyl)ethyl)-N⁴-((1r,4r)-4-hydroxycyclohexyl)-N²-methylfuran-2,4-dicarboxamide

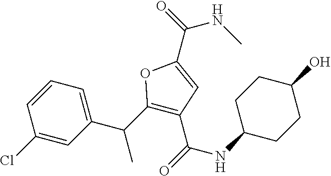 [SMILES: CNC(=O)c1cc(C(=O)N[C@H]2CC[C@@H](O)CC2)c(C(C)c2cccc(Cl)c2)o1]

2-(1-(3-Chlorophenyl)ethyl)-5-(methylcarbamoyl)furan-3-carboxylic acid (For a preparation see Intermediate 18, 242 mg, 0.786 mmol) was dissolved in DMF (5 mL) and DIPEA (0.687 mL, 3.93 mmol) was added. (1r,4r)-4-Aminocyclohexan-1-ol (181 mg, 1.573 mmol, commercially available from, for example, Fluorochem) was added followed by HATU (449 mg, 1.180 mmol) and the reaction mixture was stirred at rt for 16 h. The reaction mixture was partitioned between ethyl acetate and saturated aq. LiCl solution. The organic layer was separated and the aqueous layer further extracted with ethyl acetate (3×30 mL). The combined organic layers were dried by passing through a hydrophobic frit and concentrated to give approximately 1 g crude brown oil. This was purified by silica gel column chromatography, eluting with 0-100% of 25% ethanol in ethyl acetate/cyclohexane over 480 mL) to give the title compound (264 mg, 0.653 mmol, 74.6% yield) as a pale pink oil.

LCMS (2 min Formic): Rt=0.89 min, [MH]⁺=405.4

### Example 3: 5-((S*)-1-(3-Chlorophenyl)ethyl)-N⁴-((1r,4S)-4-hydroxycyclohexyl)-N²-methylfuran-2,4-dicarboxamide (Single Enantiomer of Unknown Configuration at Methyl Centre)

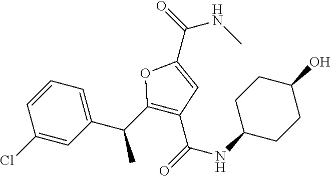 [SMILES: CNC(=O)c1cc(C(=O)N[C@H]2CC[C@@H](O)CC2)c([C@@H](C)c2cccc(Cl)c2)o1]

A sample of 5-(1-(3-chlorophenyl)ethyl)-N⁴-((1r,4r)-4-hydroxycyclohexyl)-N²-methylfuran-2,4-dicarboxamide (For a preparation see Example 2, 254 mg) was separated by chiral column chromatography using the following conditions:

Crude sample dissolved in 1 mL EtOH.

Solvent: 25% EtOH (+0.2% isopropylamine)/heptane (+0.2% isopropylamine, flow rate=30 mL/min.

Wavelength 215 nm

Column 30 mm×25 cm Chiralpak AD-H (5 μm)

The fractions corresponding to the first eluting peak were combined and evaporated to give the title compound (123 mg, 0.303 mmol).

LCMS (2 min Formic): Rt=0.89 min, [MH]⁺=405.4

¹H NMR (400 MHz, DMSO-d₆) δ ppm 8.37 (q, J=4.40 Hz, 1H) 7.91 (d, J=7.83 Hz, 1H) 7.51 (s, 1H) 7.43 (s, 1H) 7.30-7.38 (m, 2H) 7.24-7.29 (m, 1H) 5.16 (q, J=7.42 Hz, 1H) 4.51 (d, J=4.40 Hz, 1H) 3.58-3.71 (m, 1H) 3.34-3.43 (m, 1H) 2.77 (d, J=4.65 Hz, 3H) 1.70-1.88 (m, 4H) 1.62 (d, J=7.34 Hz, 3H) 1.15-1.37 (m, 4H)

### Example 4: (+/−)-N⁴-((1r,4S)-4-Hydroxycyclohexyl)-N²-methyl-5-(1-(p-tolyl)ethyl)furan-2,4-dicarboxamide

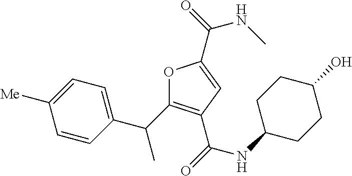 [SMILES: CNC(=O)c1cc(C(=O)N[C@H]2CC[C@H](O)CC2)c(C(C)c2ccc(C)cc2)o1]

(+/−)-5-(Methylcarbamoyl)-2-(1-(p-tolyl)ethyl)furan-3-carboxylic acid (For a preparation see Intermediate 24, 121.1 mg, 0.421 mmol) was dissolved in DMF (3 mL) and DIPEA (0.368 mL, 2.10 mmol) was added. (1r,4r)-4-Aminocyclohexan-1-ol (98 mg, 0.851 mmol) was added followed by HATU (243 mg, 0.639 mmol) and the reaction mixture was stirred at r.t. overnight. The reaction mixture was partitioned between ethyl acetate and saturated aq. LiCl solution. The organic layer was separated and the aqueous layer further extracted with ethyl acetate (3×30 mL). The combined organic layers were dried by passing through a hydrophobic frit and concentrated in vacuo to give approximately 860 mg of a pale yellow oil. This was purified by silica gel column chromatography, eluting with 0-70% of 25% ethanol in ethyl acetate/cyclohexane and concentrated in vacuo to give 130.7 mg of a colourless oil. This was further purified by MDAP (formic). The fractions containing desired product were combined and partitioned between saturated NaHCO₃ solution and ethyl acetate. The organic layer was separated and aqueous layer extracted with further ethyl acetate (3×20 mL). The combined organic layers were dried (hydrophobic frit) and concentrated in vacuo to give the title compound (41.2 mg, 0.096 mmol, 23% yield). LCMS (2 min Formic): Rt=0.86 min, [MH]⁺=385.4.

### Example 5: N⁴-((1r,4S)-4-Hydroxycyclohexyl)-N²-methyl-5-((S*)-1-(p-tolyl)ethyl)furan-2,4-dicarboxamide

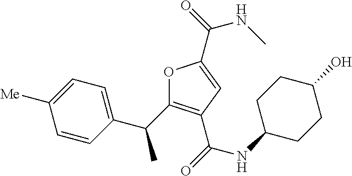 [SMILES: CNC(=O)c1cc(C(=O)N[C@H]2CC[C@H](O)CC2)c([C@@H](C)c2ccc(C)cc2)o1]

A 33 mg sample of Example 4 was separated by chiral chromatography using the following conditions: Sample was dissolved in EtOH (1 mL). This solution was injected onto the column (Column: 30 mm×25 cm Chiralpak AD-H (5 μm), Lot No ADH13231) and eluted with 30% EtOH (+0.2% isopropylamine)/heptane (+0.2% isopropylamine), flow rate=30 mL/min, wavelength, 215 nm, 4. Ref 550, 100. Fractions from the first eluting peak were combined and concentrated in vacuo and then transferred to a weighed flask to afford the title compound (14 mg, 0.036 mmol)

LCMS (2 min Formic): Rt=0.87 min, [MH]⁺=385.4

### Example 6: (+/−)-5-(1-(3-Chloro-4-methoxyphenyl)ethyl)-N⁴-((1r,4S)-4-hydroxycyclohexyl)-N²-methylfuran-2,4-dicarboxamide

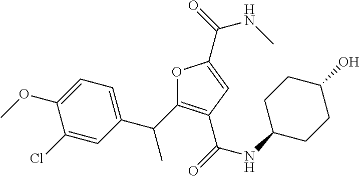 [SMILES: CNC(=O)c1cc(C(=O)N[C@H]2CC[C@H](O)CC2)c(C(C)c2ccc(OC)c(Cl)c2)o1]

(+/−)-2-(1-(3-Chloro-4-methoxyphenyl)ethyl)-5-(methylcarbamoyl)furan-3-carboxylic acid (For a preparation see Intermediate 27, 300 mg, 0.888 mmol) was dissolved in DMF (3 mL) and DIPEA (0.776 mL, 4.44 mmol) was added. (1r,4r)-4-Aminocyclohexan-1-ol (205 mg, 1.78 mmol) was added followed by HATU (507 mg, 1.33 mmol) and the reaction mixture was stirred at rt overnight. The reaction mixture was partitioned between ethyl acetate and saturated aq. LiCl solution. The organic layer was separated and the aqueous layer further extracted with ethyl acetate (3×30 mL). The combined organic layers were dried by passing through a hydrophobic frit and concentrated in vacuo to give approximately 700 mg of a pale yellow oil. This was purified by silica gel column chromatography, eluting with 0-70% of 25% ethanol in ethyl acetate/cyclohexane and concentrated in vacuo to give 283 mg of a colourless oil. This was further purified by MDAP (formic). The fractions containing the desired product were combined and partitioned between saturated NaHCO₃ solution and ethyl acetate. The organic layer was separated and the aqueous layer extracted with further ethyl acetate (3×20 mL). The combined organic layers were dried by passing through a hydrophobic frit and concentrated in vacuo to give the title compound (191.5 mg, 0.396 mmol, 45% yield). LCMS (2 min Formic): Rt=0.86 min, [MH]⁺=435.4.

### Example 7: 5-((S*)-1-(3-Chloro-4-methoxyphenyl)ethyl)-N⁴-((1r,4S)-4-hydroxycyclohexyl)-N²-methylfuran-2,4-dicarboxamide

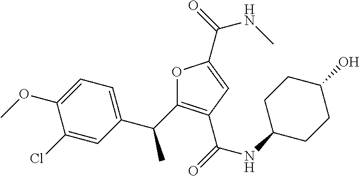 [SMILES: CNC(=O)c1cc(C(=O)N[C@H]2CC[C@H](O)CC2)c([C@@H](C)c2ccc(OC)c(Cl)c2)o1]

A 145 mg sample of Example 6 was dissolved in EtOH (1 mL) and purified by chiral column chromatography under the following conditions: Sample was injected in aliquots (0.5 mL) onto the column (column: 30 mm×25 cm Chiralpak AD-H (5 μm), Lot No ADH13231) and eluted with 30% EtOH (+0.2% isopropylamine)/heptane (+0.2% isopropylamine), flow rate=30 mL/min, wavelength, 215 nm, 4. Ref 550, 100. Total number of injections=2. Fractions from the first eluting peak were combined and concentrated in vacuo to afford the title compound (57.3 mg, 0.132 mmol).

LCMS (2 min Formic): Rt=0.86 min, [MH]⁺=435.4.

### Example 8: N⁴-((1r,4S)-4-Hydroxycyclohexyl)-N²-methyl-5-((S)-1-phenylethyl)furan-2,4-dicarboxamide

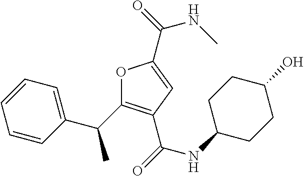 [SMILES: CNC(=O)c1cc(C(=O)N[C@H]2CC[C@H](O)CC2)c([C@@H](C)c2ccccc2)o1]

(S)-5-(Methylcarbamoyl)-2-(1-phenylethyl)furan-3-carboxylic acid (For a preparation see Intermediate 10, 100 mg, 0.366 mmol), HATU (167 mg, 0.439 mmol) and DIPEA (0.192 mL, 1.10 mmol) were stirred in DMF (2 mL) at rt for 5 min. (1r,4r)-4-Aminocyclohexan-1-ol (50.6 mg, 0.439 mmol) was added and the reaction stirred at r.t. for 2 h. The reaction was diluted with water and was extracted with EtOAc, the organic phase was washed with 10% LiCl (aq), dried using a hydrophobic frit and concentrated to a brown gum. This gum was purified by MDAP (formic method) to give the title compound (105 mg, 0.282 mmol, 77% yield).

LCMS (2 min Formic): Rt=0.81 min, [MH]⁺=371.4

¹H NMR (400 MHz, METHANOL-d₄) δ ppm 7.39 (d, J=7.34 Hz, 2H) 7.25-7.34 (m, 3H) 7.16-7.23 (m, 1H) 5.13 (q, J=7.34 Hz, 1H) 3.73-3.85 (m, 1H) 3.50-3.59 (m, 1H) 2.92 (s, 3H) 1.87-2.04 (m, 4H) 1.70 (d, J=7.34 Hz, 3H) 1.35-1.47 (m, 4H) 1.31 (d, J=6.60 Hz, 3H)

### Example 9: (+/−) 5-(1-(3-Chlorophenyl)-2-cyanoethyl)-N⁴-((1r,4r)-4-hydroxycyclohexyl)-N²-methylfuran-2,4-dicarboxamide

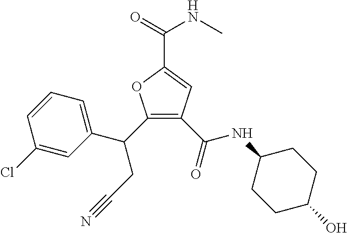 [SMILES: CNC(=O)c1cc(C(=O)N[C@H]2CC[C@H](O)CC2)c(C(CC#N)c2cccc(Cl)c2)o1]

(+/−) 2-(1-(3-Chlorophenyl)-2-cyanovinyl)-5-(methylcarbamoyl)furan-3-carboxylic acid (For a preparation see Intermediate 30, 80 mg, 0.242 mmol), DIPEA (63.4 μL, 0.363 mmol) and HATU (138 mg, 0.363 mmol) were dissolved in DMF (1.2 mL) and the mixture stirred at r.t. for 15 min. (1r,4r)-4-Aminocyclohexan-1-ol (41.8 mg, 0.363 mmol) was added and the reaction stirred at rt for 1 h. The reaction was diluted with EtOAc (40 mL) and washed with 2M HCl (aq) (5 mL). The aqueous was extracted with EtOAc (10 mL) and the combined organics passed through a hydrophobic frit and concentrated in vacuo. The residue was purified by silica gel chromatography (30-80% of a 25% EtOH in EtOAc mixture/TBME) and the appropriate fractions were combined and concentrated. The residue was transferred to a vial in DCM/MeOH and concentrated under a positive pressure of N₂ to give a brown oil. The brown oil was dissolved in dry IPA (1.2 mL) and sodium borohydride (165 mg, 4.35 mmol) added. The reaction was stirred at r.t. for 1 h. Additional sodium borohydride (165 mg, 4.35 mmol) was added and the reaction stirred at r.t. for 4.5 h, after which the reaction was quenched with acetic acid (2 mL) and MeOH (2 mL). The solvents were concentrated under a positive pressure of N₂ and the residue dried in vacuo. The residue was purified by MDAP (Formic) to give the title compound as a white solid. (41 mg, 0.095 mmol, 39.4% yield)

LCMS (2 min Formic): Rt=0.82 min, [MH]⁺=430.4

### Example 10: 5-((S)-1-(3-Chlorophenyl)-2-cyanoethyl)-N⁴-((1r,4S)-4-hydroxycyclohexyl)-N²-methylfuran-2,4-dicarboxamide

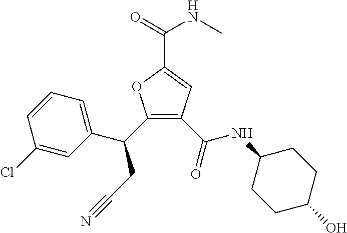 [SMILES: CNC(=O)c1cc(C(=O)N[C@H]2CC[C@H](O)CC2)c([C@@H](CC#N)c2cccc(Cl)c2)o1]

A sample of Example 9 (41 mg) was purified by chiral column chromatography. The racemate was dissolved in EtOH (2 mL). Injection: 2 mL of the solution was injected onto the column (30% EtOH (+0.2% isopropylamine)/heptane (+0.2% isopropylamine), flow rate=30 mL/min, detection wavelength=215 nm, 4. Ref 550, 100, Column 3 cm×25 cm Chiralpak AD-H (5 μm), lot no. ADH13231). Total number of injections=1. The fractions corresponding to the first eluting peak were collected to afford the title compound (15 mg, 0.035 mmol)

LCMS (2 min Formic): Rt=0.82 min, [MH]⁺=430.3

### Example 11: (+/−)-5-((3-Chlorophenyl)(methoxy)methyl)-N⁴-((1r,4r)-4-hydroxycyclohexyl)-N²-methylfuran-2,4-dicarboxamide

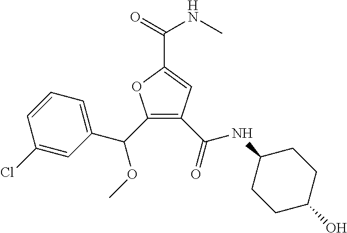 [SMILES: CNC(=O)c1cc(C(=O)N[C@H]2CC[C@H](O)CC2)c(C(OC)c2cccc(Cl)c2)o1]

To an oven dried 20 mL microwave vial was added diacetoxypalladium (20.5 mg, 0.091 mmol), Xantphos (52.9 mg, 0.091 mmol), (1r,4r)-4-aminocyclohexan-1-ol (316 mg, 2.74 mmol), DMAP (134 mg, 1.098 mmol) and cobalt carbonyl (with 1-10% hexane, 174 mg, 0.457 mmol). The vial was sealed and purged with nitrogen. A degassed solution of 4-bromo-5-((3-chlorophenyl)(methoxy)methyl)-N-methylfuran-2-carboxamide (For a preparation see Intermediate 32, 400 mg, 0.915 mmol) in 1,4-dioxane (9146 μL) was added and the reaction irradiated by microwave at 90° C. for 3 h and stood at rt overnight. The reaction was filtered through Celite, washing with EtOAc (60 mL) and the collected solution concentrated in vacuo. The residue was dissolved in DCM (with minimal MeOH added to ease solution) and purified by silica (120 g) gel chromatography eluting on a gradient of 40-100% 3:1 EtOAc:EtOH. The fractions corresponding to the major eluting peak were combined and concentrated to give a brown residue. This was dissolved in DMSO:MeOH (50%, 3 mL) and purified by MDAP (high pH). The relevant fractions were combined and concentrated to give the title compound (162 mg, 0.385 mmol, 42% yield) as a white solid.

LCMS (2 min Formic): Rt=0.85 min, [M-OMe]⁺=389.3

### Example 12: 5-((S*)-(3-chlorophenyl)(methoxy)methyl)-N⁴-((1r,4S)-4-hydroxycyclohexyl)-N²-methylfuran-2,4-dicarboxamide

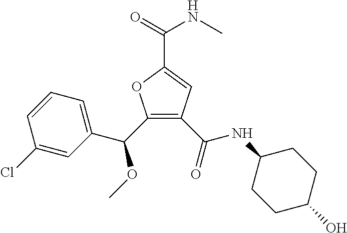 [SMILES: CNC(=O)c1cc(C(=O)N[C@H]2CC[C@H](O)CC2)c([C@@H](OC)c2cccc(Cl)c2)o1]

A sample of Example 11 (159 mg) was purified by chiral HPLC. The racemate was dissolved in EtOH (1 mL). Injection: 0.25 mL of the solution was injected onto the column (20% EtOH (+0.2% isopropylamine)/heptane (+0.2% isopropylamine), flow rate=30 mL/min, detection wavelength=215 nm, 4. Ref 550, 100, Column 30 mm×25 cm Chiralpak AD-H (5 μm)). Total number of injections=5. The fractions corresponding to the first eluting peak were collected and concentrated in vacuo to afford the title compound (58 mg, 0.138 mmol).

LCMS (2 min High pH): Rt=0.85 min, [MH]⁺=421.4.

### Example 13: N⁴-((1R,5S,6r)-3-oxabicyclo[3.1.0]hexan-6-yl)-N²-methyl-5-((S)-1-phenylethyl)furan-2,4-dicarboxamide

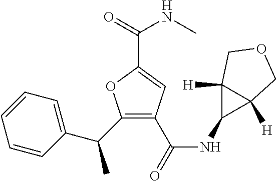 [SMILES: CNC(=O)c1cc(C(=O)N[C@H]2[C@@H]3COC[C@@H]32)c([C@@H](C)c2ccccc2)o1]

(S)-5-(Methylcarbamoyl)-2-(1-phenylethyl)furan-3-carboxylic acid (For a preparation see Intermediate 10, 94.5 mg, 0.346 mmol) was taken up in DMF (3 mL). DIPEA (0.181 mL, 1.04 mmol), then (1R,5S,6r)-3-oxabicyclo[3.1.0]hexan-6-amine hydrochloride (56.3 mg, 0.415 mmol) were added, and the reaction left to stir for 10 min. HATU (197 mg, 0.519 mmol) was added, and the reaction left to stir at room temperature for 1.5 hours. The reaction was concentrated in vacuo. The residue was partitioned between ethyl acetate (10 mL) and sodium bicarbonate (sat. aq. 10 mL). The organic was washed with 1M HCl (10 mL), brine (10 mL), dried with Na₂SO₄, filtered and concentrated in vacuo to yield a brown solid. The crude product was applied to a 10 g silica cartridge in the minimum of DCM (and a couple of drops of MeOH) and eluted with 5-50% (3:1 EtOAc:EtOH) in cyclohexane. The appropriate fractions were combined and concentrated in vacuo. The aqueous layers were each further extracted with ethyl acetate (10 mL for each). The combined organics were dried with Na₂SO₄, filtered and concentrated in vacuo to yield a yellow gum. The crude product was applied to a 10 g ULTRA silica SNAP cartridge in the minimum of DCM (and a couple of drops of MeOH) and eluted with 5-50% (3:1 EtOAc:EtOH) in cyclohexane. The appropriate fractions were combined and concentrated in vacuo. The two residues from chromatography were combined to yield the title compound (67 mg, 0.180 mmol, 52% yield) as a white solid. LCMS (2 min Formic): Rt=0.83 min, [MH]⁺=355.5.

¹H NMR (400 MHz, DMSO-d₆) δ ppm 7.37-7.45 (m, 3H) 7.30 (t, J=7.46 Hz, 2H) 7.16-7.24 (m, 1H) 5.17 (q, J=7.25 Hz, 1H) 3.84 (dd, J=8.31, 2.69 Hz, 2H) 3.59-3.66 (m, 2H) 3.27 (s, 1H) 3.18 (d, J=5.14 Hz, 1H) 2.74-2.79 (m, 3H) 1.81-1.88 (m, 2H) 1.62 (d, J=7.34 Hz, 3H) 1.10 (t, J=6.97 Hz, 1H)

### Example 14: (S)—N²-methyl-5-(1-phenylethyl)-N⁴-propylfuran-2,4-dicarboxamide

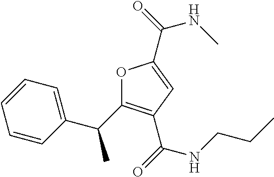 [SMILES: CCCNC(=O)c1cc(C(=O)NC)oc1[C@@H](C)c1ccccc1]

To a mixture of (S)-5-(methylcarbamoyl)-2-(1-phenylethyl)furan-3-carboxylic acid (For a preparation see Intermediate 10, 51.1 mg, 0.187 mmol) and N,N-diisopropylethylamine (0.098 mL, 0.561 mmol) in DMF (0.8 mL) was added propan-1-amine (0.046 mL, 0.561 mmol) and HATU (110.9 mg, 0.292 mmol). The resulting yellow solution was stirred at room temperature for 3.75 hr. The reaction mixture was then concentrated under a stream of nitrogen giving a thick yellow oil. The oil was diluted with acetonitrile to make up a volume of 1 mL, which was then purified by MDAP (1×1 mL injection, formic). The desired fraction had the solvent evaporated in vacuo to give a residue which was then dissolved in acetonitrile (˜4-5 mL), transferred to a vial, concentrated under a stream of nitrogen and the residue dried in a vacuum oven for 6 hours resulting in a colourless glass which solidified upon scraping to give the title compound as a white solid (29.4 mg, 0.094 mmol, 50.0% yield). LCMS (2 min Formic) Rt=0.95 min, [MH]⁺=315.3.

### Example 15: (+/−) N⁴-((1r,4r)-4-Hydroxycyclohexyl)-N²-methyl-5-(1-(6-methylpyridin-2-yl)ethyl)furan-2,4-dicarboxamide

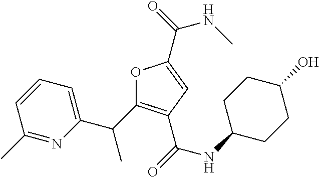 [SMILES: CNC(=O)c1cc(C(=O)N[C@H]2CC[C@H](O)CC2)c(C(C)c2cccc(C)n2)o1]

N⁴-((1r,4r)-4-Hydroxycyclohexyl)-N²-methyl-5-(1-(6-methylpyridin-2-yl)vinyl)furan-2,4-dicarboxamide (For a preparation see Intermediate 21, 123 mg, 0.321 mmol) was dissolved in methanol (10 mL) and hydrogenated in a Thales H-cube over a 5% Pd/C Catcart (34.1 mg, 0.321 mmol) at 1 mL/min flow rate, room temperature and atmospheric pressure for 20 mins. The reaction mixture concentrated to give the title compound (68 mg, 0.177 mmol, 44.0% yield) as a white solid.

LCMS (2 min Formic): Rt=0.43 min, [MH]⁺=386.4

### Example 16: N⁴-((1r,4S)-4-Hydroxycyclohexyl)-N²-methyl-5-((S*)-1-(6-methylpyridin-2-yl)ethyl)furan-2,4-dicarboxamide (Single Enantiomer of Unknown Configuration at Methyl Centre)

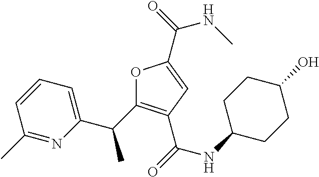 [SMILES: CNC(=O)c1cc(C(=O)N[C@H]2CC[C@H](O)CC2)c([C@@H](C)c2cccc(C)n2)o1]

60 mg of N⁴-((1r,4r)-4-hydroxycyclohexyl)-N²-methyl-5-(1-(6-methylpyridin-2-yl)ethyl)furan-2,4-dicarboxamide (For a preparation see Example 15) was separated by chiral column chromatography using the following conditions:

Crude sample dissolved in 1 mL EtOH.

Injection; 1 mL of the solution was injected onto the column.

Solvent: 20% EtOH (+0.2% isopropylamine)/heptane (+0.2% isopropylamine), flow rate=30 mL/min. Wavelength 215 nm

Column 30 mm×25 cm Chiralpak AD-H (5 μm)

Total number of injections 1

The fractions corresponding to the first eluting peak were combined and evaporated to give the title compound (13 mg).

LCMS (2 min Formic): Rt=0.43 min, [MH]⁺=386.3

### Example 17: (+/−) 5-(1-(1H-indol-4-yl)ethyl)-N⁴-((1r,4r)-4-hydroxycyclohexyl)-N²-methylfuran-2,4-dicarboxamide

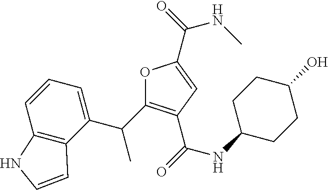 [SMILES: CNC(=O)c1cc(C(=O)N[C@H]2CC[C@H](O)CC2)c(C(C)c2cccc3[nH]ccc23)o1]

2-(1-(1H-Indol-4-yl)ethyl)-5-(methylcarbamoyl)furan-3-carboxylic acid (For a preparation see Intermediate 35, 587 mg, 1.879 mmol) and HATU (1072 mg, 2.82 mmol) were dissolved in DCM (15 mL). DIPEA (0.657 mL, 3.76 mmol) was added and the mixture was stirred for 15 mins. Then, (1r,4r)-4-aminocyclohexan-1-ol (325 mg, 2.82 mmol) was added and the reaction was stirred for 2 hours. The reaction was stopped, partitioned between DCM and saturated aqueous sodium bicarbonate solution. The aqueous layer was washed with DCM. The organic layers were combined, dried over Na₂SO₄ and concentrated in vacuo. The crude product was applied to a 25 g silica cartridge in the minimum of DCM+Methanol and eluted with 0% (3:1 EtOAc:EtOH) in cyclohexane for 1 column volume then 0-100% (3:1 EtOAc:EtOH) over 12 column volumes then held at 100% for 2 column volumes. Desired fractions were concentrated in vacuo to give crude title compound as a red oil. The crude product was applied to a 25 g silica S cartridge in the minimum of DCM and eluted with 0% (3:1 EtOAc:EtOH) in cyclohexane for 2 column volumes then 0-12% (3:1 EtOAc:EtOH) over 10 column volumes then held at 100% for 3 column volumes. Desired fractions were combined and concentrated in vacuo to give the title compound (670 mg, 1.59 mmol, 84% yield) as a pale orange solid.

LCMS (2 min Formic): Rt=0.75 min, [MH]⁺=410.5

### Examples 18-111

Examples 18-111 were prepared in an analogous manner to the previous examples

| Ex | | | | Rt |
|---|---|---|---|---|
| No. | Name | Structure | [MH]⁺ | (min) |
| 18 | 5-((S*)-1-(3-Chloro-4- methylphenyl)ethyl)-N⁴-((1r,4S)- 4-hydroxycyclohexyl)-N²- methylfuran-2,4-dicarboxamide | 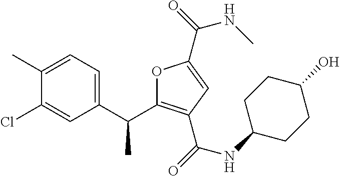 [SMILES: CNC(=O)c1cc(C(=O)N[C@H]2CC[C@H](O)CC2)c([C@@H](C)c2ccc(C)c(Cl)c2)o1] | 419.3 | 0.96 (formic) |
| 19 | N⁴-(1R,5S,6r)-3- Oxabicyclo[3.1.0]hexan-6-yl)-5- ((S*)-1-(3-chlorophenyl)ethyl)- N²-methylfuran-2,4- dicarboxamide | 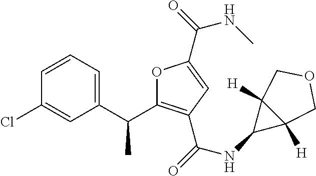 [SMILES: CNC(=O)c1cc(C(=O)N[C@H]2[C@@H]3COC[C@@H]32)c([C@@H](C)c2cccc(Cl)c2)o1] | 389.3 | 0.93 (formic) |
| 20 | N⁴-((1r,4S)-4- Methoxycyclohexyl)-N²-methyl- 5-((S*)-1-phenylethyl)furan-2,4- dicarboxamide | 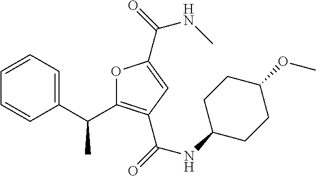 [SMILES: CNC(=O)c1cc(C(=O)N[C@H]2CC[C@H](OC)CC2)c([C@@H](C)c2ccccc2)o1] | 385.4 | 0.94 (formic) |
| 21 | N⁴-((1r,4S)-4- Hydroxycyclohexyl)-5-((S*)-1- (4-methoxy-3- methylphenyl)ethyl)-N²- methylfuran-2,4-dicarboxamide | 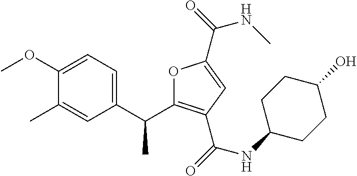 [SMILES: CNC(=O)c1cc(C(=O)N[C@H]2CC[C@H](O)CC2)c([C@@H](C)c2ccc(OC)c(C)c2)o1] | 415.4 | 0.87 (formic) |
| 22 | 5-(1-(3,5-Dichlorophenyl)ethyl)- N⁴-((1r,4r)-4- hydroxycyclohexyl)-N²- methylfuran-2,4-dicarboxamide | 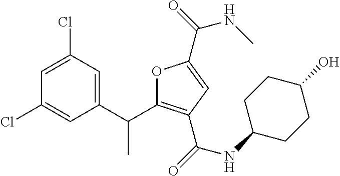 [SMILES: CNC(=O)c1cc(C(=O)N[C@H]2CC[C@H](O)CC2)c(C(C)c2cc(Cl)cc(Cl)c2)o1] | 439.3 | 1.00 (formic) |
| 23 | 5-((S*)-1-(3,5- Dichlorophenyl)ethyl)-N⁴- ((1r,4S)-4-hydroxycyclohexyl)- N²-methylfuran-2,4- dicarboxamide | 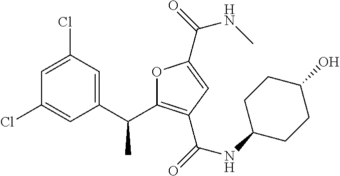 [SMILES: CNC(=O)c1cc(C(=O)N[C@H]2CC[C@H](O)CC2)c([C@@H](C)c2cc(Cl)cc(Cl)c2)o1] | 439.4 | 1.00 (formic) |
| 24 | N⁴-(1R,5S,6r)-3- Oxabicyclo[3.1.0]hexan-6-yl)-5- ((S*)-1-(3-chlorophenyl)-2- cyanoethyl)-N²-methylfuran-2,4- dicarboxamide | 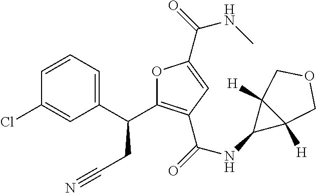 [SMILES: CNC(=O)c1cc(C(=O)N[C@H]2[C@@H]3COC[C@@H]32)c([C@@H](CC#N)c2cccc(Cl)c2)o1] | 414.3 | 0.88 (formic) |
| 25 | N⁴-((1r,4S)-4- Hydroxycyclohexyl)-N²-methyl- 5-((S*)-1-(m-tolyl)ethyl)furan- 2,4-dicarboxamide | 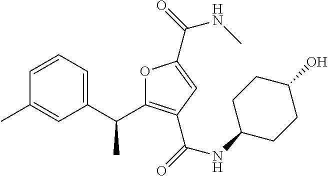 [SMILES: CNC(=O)c1cc(C(=O)N[C@H]2CC[C@H](O)CC2)c([C@@H](C)c2cccc(C)c2)o1] | 385.4 | 0.86 (formic) |
| 26 | N⁴-(1R,5S,6r)-3- Oxabicyclo[3.1.0]hexan-6-yl)-5- (1-(3-chlorophenyl)ethyl)-N²- methylfuran-2,4-dicarboxamide | 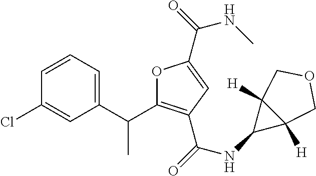 [SMILES: CNC(=O)c1cc(C(=O)N[C@H]2[C@@H]3COC[C@@H]32)c(C(C)c2cccc(Cl)c2)o1] | 389.3 | 0.93 (formic) |
| 27 | 5-(1-(3-Chloro-5- methoxyphenyl)ethyl)-N⁴- ((1r,4r)-4-hydroxycyclohexyl)- N²-methylfuran-2,4- dicarboxamide | 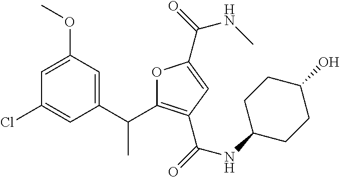 [SMILES: CNC(=O)c1cc(C(=O)N[C@H]2CC[C@H](O)CC2)c(C(C)c2cc(Cl)cc(OC)c2)o1] | 435.4 | 0.91 (formic) |
| 28 | N⁴-((1r,4S)-4- Hydroxycyclohexyl)-5-((S*)-1- (4-methoxyphenyl)ethyl)-N²- methylfuran-2,4-dicarboxamide | 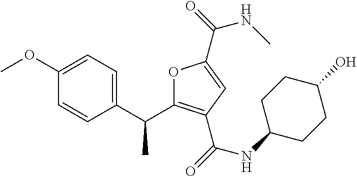 [SMILES: CNC(=O)c1cc(C(=O)N[C@H]2CC[C@H](O)CC2)c([C@@H](C)c2ccc(OC)cc2)o1] | 401.4 | 0.80 (formic) |
| 29 | 5-((S*)-1-(3-Chloro-5- methoxyphenyl)ethyl)-N⁴- ((1r,4S)-4-hydroxycyclohexyl)- N²-methylfuran-2,4- dicarboxamide | 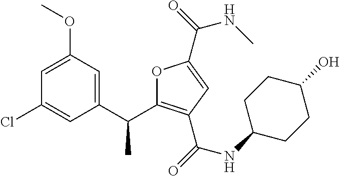 [SMILES: CNC(=O)c1cc(C(=O)N[C@H]2CC[C@H](O)CC2)c([C@@H](C)c2cc(Cl)cc(OC)c2)o1] | 435.4 | 0.91 (formic) |
| 30 | N⁴-((1R,3S,5S,6r)-3- Hydroxybicyclo[3.1.0]hexan-6- yl)-N²-methyl-5-((S)-1- phenylethyl)furan-2,4- dicarboxamide | 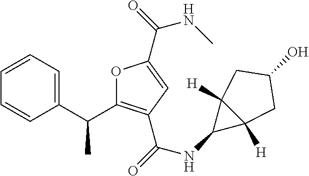 [SMILES: CNC(=O)c1cc(C(=O)N[C@H]2[C@@H]3C[C@H](O)C[C@@H]32)c([C@@H](C)c2ccccc2)o1] | 369.2 | 0.81 (formic) |
| 31 | 5-((S*)-1-(2- Chlorophenyl)ethyl)-N⁴-((1r,4S)- 4-hydroxycyclohexyl)-N²- methylfuran-2,4-dicarboxamide | 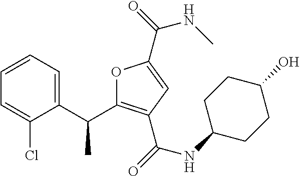 [SMILES: CNC(=O)c1cc(C(=O)N[C@H]2CC[C@H](O)CC2)c([C@@H](C)c2ccccc2Cl)o1] | 405.4 | 0.84 (formic) |
| 32 | 5-(1-(3-Chloro-4- methylphenyl)ethyl)-N⁴-((1r,4r)- 4-hydroxycyclohexyl)-N²- methylfuran-2,4-dicarboxamide | 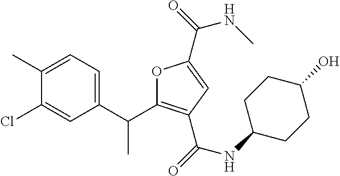 [SMILES: CNC(=O)c1cc(C(=O)N[C@H]2CC[C@H](O)CC2)c(C(C)c2ccc(C)c(Cl)c2)o1] | 419.4 | 0.95 (formic) |
| 33 | 5-((S*)-1-(4- Chlorophenyl)ethyl)-N⁴-((1r,4S)- 4-hydroxycyclohexyl)-N²- methylfuran-2,4-dicarboxamide | 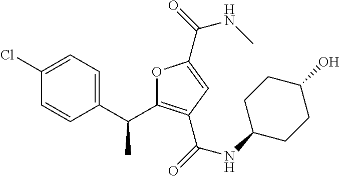 [SMILES: CNC(=O)c1cc(C(=O)N[C@H]2CC[C@H](O)CC2)c([C@@H](C)c2ccc(Cl)cc2)o1] | 405.3 | 0.94 (High pH) |
| 34 | N⁴-((1R,3r,5S,6r)-3- Hydroxybicyclo[3.1.0]hexan-6- yl)-N²-methyl-5-(1- phenylethyl)furan-2,4- dicarboxamide | 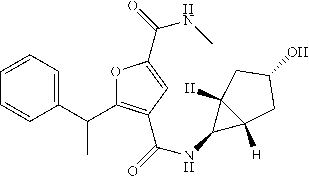 [SMILES: CNC(=O)c1cc(C(=O)N[C@H]2[C@@H]3C[C@H](O)C[C@@H]32)c(C(C)c2ccccc2)o1] | 369.3 | 0.81 (formic) |
| 35 | 5-(1-(2-Chlorophenyl)ethyl)-N⁴- ((1r,4r)-4-hydroxycyclohexyl)- N²-methylfuran-2,4- dicarboxamide | 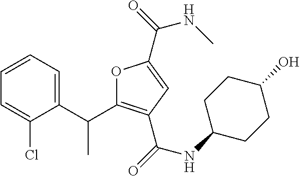 [SMILES: CNC(=O)c1cc(C(=O)N[C@H]2CC[C@H](O)CC2)c(C(C)c2ccccc2Cl)o1] | 405.3 | 0.84 (formic) |
| 36 | 5-((S*)-2-Cyano-1-phenylethyl)- N⁴-((1r,4S)-4- hydroxycyclohexyl)-N²- methylfuran-2,4-dicarboxamide | 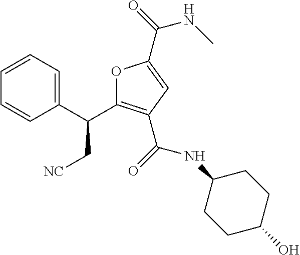 [SMILES: CNC(=O)c1cc(C(=O)N[C@H]2CC[C@H](O)CC2)c([C@@H](CC#N)c2ccccc2)o1] | 396.3 | 0.73 (formic) |
| 37 | (S*)-N⁴-Cyclopentyl-N²-methyl- 5-(1-phenylethyl)furan-2,4- dicarboxamide | 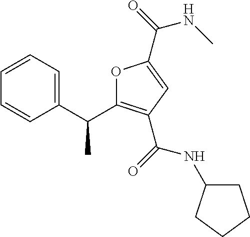 [SMILES: CNC(=O)c1cc(C(=O)NC2CCCC2)c([C@@H](C)c2ccccc2)o1] | 341.3 | 1.04 (formic) |
| 38 | N²-Methyl-N⁴-((1S,2S)-2- methylcyclopropyl)-5-((S*)-1- phenylethyl)furan-2,4- dicarboxamide | 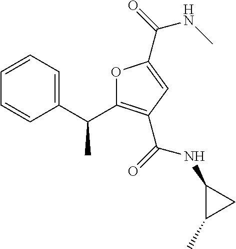 [SMILES: CNC(=O)c1cc(C(=O)N[C@H]2C[C@@H]2C)c([C@@H](C)c2ccccc2)o1] | 327.2 | 0.97 (formic) |
| 39 | (S)-N⁴-Cyclobutyl-N²-methyl-5- (1-phenylethyl)furan-2,4- dicarboxamide | 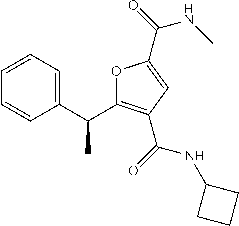 [SMILES: CNC(=O)c1cc(C(=O)NC2CCC2)c([C@@H](C)c2ccccc2)o1] | 327.1 | 0.99 (formic) |
| 40 | (+/-)-N⁴-((1R,5S,6r)-3- Oxabicyclo[3.1.0]hexan-6-yl)- N²-methyl-5-(1- phenylethyl)furan-2,4- dicarboxamide | 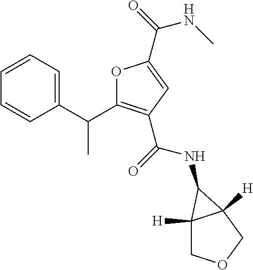 [SMILES: CNC(=O)c1cc(C(=O)N[C@H]2[C@@H]3COC[C@@H]32)c(C(C)c2ccccc2)o1] | 355.3 | 0.83 (formic) |
| 41 | (+/-)-N⁴-((1r,4r)-4- Methoxycyclohexyl)-N²-methyl- 5-(1-phenylethyl)furan-2,4- dicarboxamide | 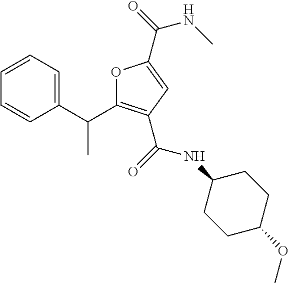 [SMILES: CNC(=O)c1cc(C(=O)N[C@H]2CC[C@H](OC)CC2)c(C(C)c2ccccc2)o1] | 385.4 | 0.94 (formic) |
| 42 | (S)-N⁴-(2-Cyclopropylethyl)-N²- methyl-5-(1-phenylethyl)furan- 2,4-dicarboxamide | 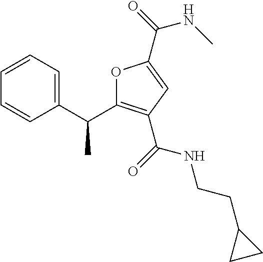 [SMILES: CNC(=O)c1cc(C(=O)NCCC2CC2)c([C@@H](C)c2ccccc2)o1] | 341.2 | 1.03 (formic) |
| 43 | N⁴-((1r,4S)-4- Hydroxycyclohexyl)-5-((S)- methoxy(phenyl)methyl)-N²- methylfuran-2,4-dicarboxamide | 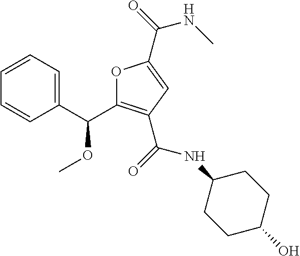 [SMILES: CNC(=O)c1cc(C(=O)N[C@H]2CC[C@H](O)CC2)c([C@@H](OC)c2ccccc2)o1] | 355.3 | 0.75 (formic) |
| 44 | (+/-)-N⁴-((1r,4r)-4- Hydroxycyclohexyl)-5-(1-(4- methoxy-3-methylphenyl)ethyl)- N²-methylfuran-2,4- dicarboxamide | 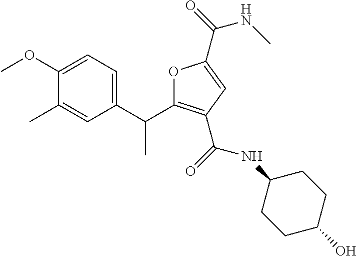 [SMILES: CNC(=O)c1cc(C(=O)N[C@H]2CC[C@H](O)CC2)c(C(C)c2ccc(OC)c(C)c2)o1] | 415.4 | 0.87 (formic) |
| 45 | (+/-)-5-(2-Cyano-1- phenylethyl)-N⁴-((1r,4r)-4- hydroxycyclohexyl)-N²- methylfuran-2,4-dicarboxamide | 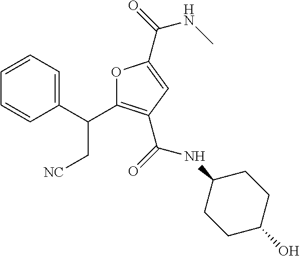 [SMILES: CNC(=O)c1cc(C(=O)N[C@H]2CC[C@H](O)CC2)c(C(CC#N)c2ccccc2)o1] | 396.4 | 0.73 (formic) |
| 46 | N⁴-((1r,4S)-4- Hydroxycyclohexyl)-5-((S*)-1- (3-methoxyphenyl)ethyl)-N²- methylfuran-2,4-dicarboxamide | 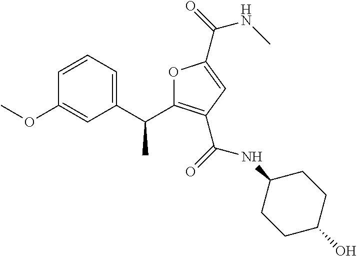 [SMILES: CNC(=O)c1cc(C(=O)N[C@H]2CC[C@H](O)CC2)c([C@@H](C)c2cccc(OC)c2)o1] | 401.4 | 0.80 (formic) |
| 47 | (S*)-N⁴-Cyclopropyl-N²-methyl- 5-(1-phenylethyl)furan-2,4- dicarboxamide | 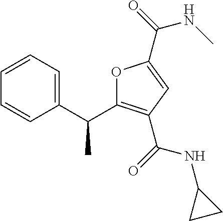 [SMILES: CNC(=O)c1cc(C(=O)NC2CC2)c([C@@H](C)c2ccccc2)o1] | 313.3 | 0.87 (formic) |
| 48 | (S)-N⁴-Isobutyl-N²-methyl-5-(1- phenylethyl)furan-2,4- dicarboxamide | 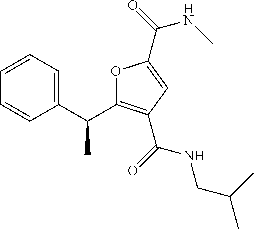 [SMILES: CNC(=O)c1cc(C(=O)NCC(C)C)c([C@@H](C)c2ccccc2)o1] | 329.3 | 1.03 (formic) |
| 49 | (+/-)-5-(1-(4- Chlorophenyl)ethyl)-N⁴-((1r,4r)- 4-hydroxycyclohexyl)-N²- methylfuran-2,4-dicarboxamide | 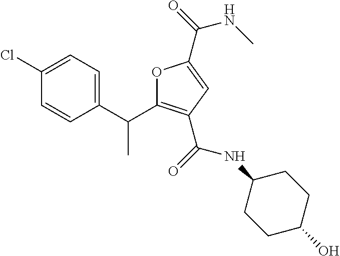 [SMILES: CNC(=O)c1cc(C(=O)N[C@H]2CC[C@H](O)CC2)c(C(C)c2ccc(Cl)cc2)o1] | 405.5 | 0.91 (formic) |
| 50 | (+/-)-N⁴-(1R,5S,6r)-3- Oxabicyclo[3.1.0]hexan-6-yl)-5- (1-(3-chlorophenyl)-2- cyanoethyl)-N²-methylfuran-2,4- dicarboxamide | 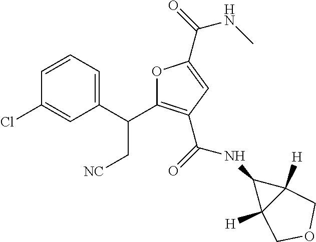 [SMILES: CNC(=O)c1cc(C(=O)N[C@H]2[C@@H]3COC[C@@H]32)c(C(CC#N)c2cccc(Cl)c2)o1] | 414.4 | 0.86 (formic) |
| 51 | (S*)-N⁴-Ethyl-N²-methyl-5-(1- phenylethyl)furan-2,4- dicarboxamide | 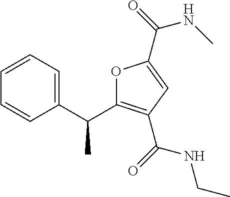 [SMILES: CCNC(=O)c1cc(C(=O)NC)oc1[C@@H](C)c1ccccc1] | 301.3 | 0.86 (formic) |
| 52 | (+/-)-N⁴-(1r,4r)-4- Hydroxycyclohexyl)-N²-methyl- 5-(1-(m-tolyl)ethyl)furan-2,4- dicarboxamide | 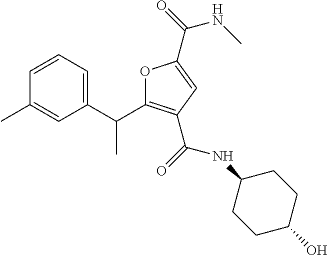 [SMILES: CNC(=O)c1cc(C(=O)N[C@H]2CC[C@H](O)CC2)c(C(C)c2cccc(C)c2)o1] | 385.4 | 0.86 (formic) |
| 53 | 5-((R)-2-Hydroxy-1- phenylethyl)-N⁴-((1r,4R)-4- hydroxycyclohexyl)-N²- methylfuran-2,4-dicarboxamide | 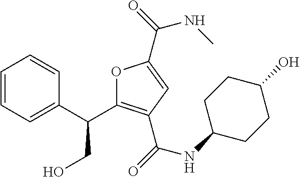 [SMILES: CNC(=O)c1cc(C(=O)N[C@H]2CC[C@H](O)CC2)c([C@@H](CO)c2ccccc2)o1] | 387.4 | 0.64 (formic) |
| 54 | (+/-)-N²-methyl-N⁴-((1S,2S)-2- Methylcyclopropyl)-5-((S)-1- phenylethyl)furan-2,4- dicarboxamide | 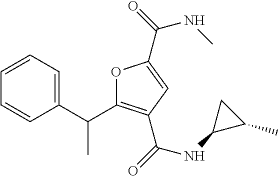 [SMILES: CNC(=O)c1cc(C(=O)N[C@H]2C[C@@H]2C)c(C(C)c2ccccc2)o1] | 327.3 | 0.97 (formic) |
| 55 | (+/-)-N⁴-((1r,4r)-4- Hydroxycyclohexyl)-N²-methyl- 5-(1-phenylethyl)furan-2,4- dicarboxamide | 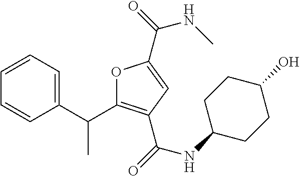 [SMILES: CNC(=O)c1cc(C(=O)N[C@H]2CC[C@H](O)CC2)c(C(C)c2ccccc2)o1] | 371.3 | 0.79 (formic) |
| 56 | (+/-)-N⁴-((1S,3S,4S)-4-hydroxy- 3-methylcyclohexyl)-N²-methyl- 5-(1-phenylethyl)furan-2,4- dicarboxamide, mixture of diasteromers | 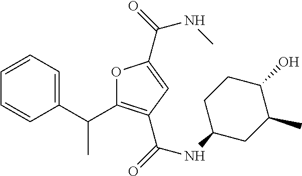 [SMILES: CNC(=O)c1cc(C(=O)N[C@H]2CC[C@H](O)[C@@H](C)C2)c(C(C)c2ccccc2)o1] | 385.4 | 0.85 (formic) |
| 57 | (+/-)-5-(2-Hydroxy-1- phenylethyl)-N⁴-((1r,4r)-4- hydroxycyclohexyl)-N²- methylfuran-2,4-di- carboxamide | 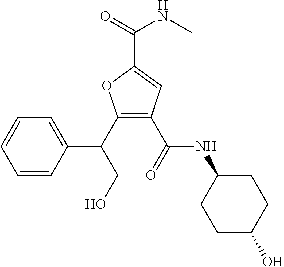 [SMILES: CNC(=O)c1cc(C(=O)N[C@H]2CC[C@H](O)CC2)c(C(CO)c2ccccc2)o1] | 387.4 | 0.66 (formic) |
| 58 | (+/-)-5-((3- Chlorophenyl)(methoxy)methyl)- N⁴-(1r,4r)-4- hydroxycyclohexyl)-N²- methylfuran- 2,4-dicarboxamide | 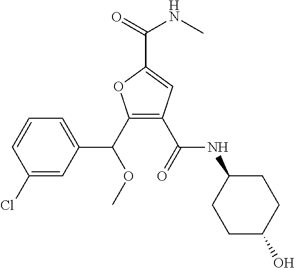 [SMILES: CNC(=O)c1cc(C(=O)N[C@H]2CC[C@H](O)CC2)c(C(OC)c2cccc(Cl)c2)o1] | 387.4 | 0.85 (formic) |
| 59 | (+/-)-N⁴-((1r,4r)-4- Hydroxycyclohexyl)-5-(1-(3- methoxyphenyl)ethyl)-N²- methylfuran-2,4-dicarboxamide | 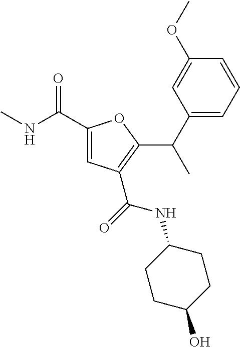 [SMILES: CNC(=O)c1cc(C(=O)N[C@H]2CC[C@H](O)CC2)c(C(C)c2cccc(OC)c2)o1] | 401.4 | 0.79 (formic) |
| 60 | (+/-)-5-(1-(3-Chlorophenyl)-2- hydroxyethyl)-N⁴-((1r,4r)-4- hydroxycyclohexyl)-N²- methylfuran-2,4-dicarboxamide | 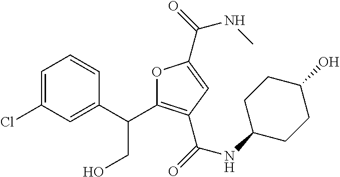 [SMILES: CNC(=O)c1cc(C(=O)N[C@H]2CC[C@H](O)CC2)c(C(CO)c2cccc(Cl)c2)o1] | 420.1 | 0.75 (formic) |
| 61 | (+/-)-N⁴-((1r,4r)-4- hydroxycyclohexyl)-5-(1-(4- methoxyphenyl)ethyl)-N²- methylfuran-2,4-dicarboxamide | 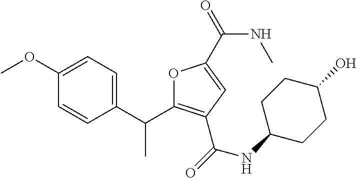 [SMILES: CNC(=O)c1cc(C(=O)N[C@H]2CC[C@H](O)CC2)c(C(C)c2ccc(OC)cc2)o1] | 401.4 | 0.78 (formic) |
| 62 | 5-((S)-1-(3-Fluorophenyl)ethyl)- N⁴-((1r,4S)-4- hydroxycyclohexyl)-N²- methylfuran-2,4-dicarboxamide | 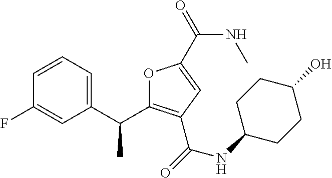 [SMILES: CNC(=O)c1cc(C(=O)N[C@H]2CC[C@H](O)CC2)c([C@@H](C)c2cccc(F)c2)o1] | 389.4 | 0.81 (formic) |
| 63 | (+/-) 5-((3- Chlorophenyl)(hydroxy)methyl)- N⁴-((1r,4r)-4- hydroxycyclohexyl)-N²- methylfuran-2,4-dicarboxamide | 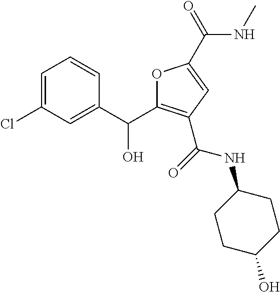 [SMILES: CNC(=O)c1cc(C(=O)N[C@H]2CC[C@H](O)CC2)c(C(O)c2cccc(Cl)c2)o1] | 407.3 | 0.75 (formic) |
| 64 | 5-((S)-1-(3,4- Dimethoxyphenyl)ethyl)-N⁴- ((1r,4S)-4-hydroxycyclohexyl)- N²-methylfuran-2,4- dicarboxamide | 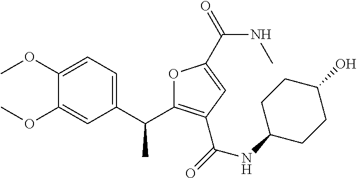 [SMILES: CNC(=O)c1cc(C(=O)N[C@H]2CC[C@H](O)CC2)c([C@@H](C)c2ccc(OC)c(OC)c2)o1] | 431.1 | 0.72 (formic) |
| 65 | (+/-)-N⁴-Cyclopropyl-N²-methyl- 5-(1-phenylethyl)furan-2,4- dicarboxamide | 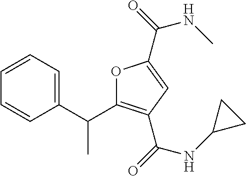 [SMILES: CNC(=O)c1cc(C(=O)NC2CC2)c(C(C)c2ccccc2)o1] | 313.1 | 0.87 (formic) |
| 66 | (+/-)-N⁴-((1R,5S,6r)-3- Oxabicyclo[3.1.0]hexan-6-yl)-5- (methoxy(phenyl)methyl)-N²- methylfuran-2,4-dicarboxamide | 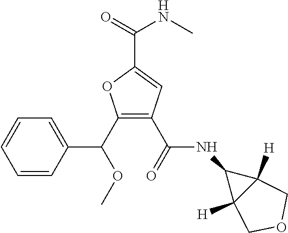 [SMILES: CNC(=O)c1cc(C(=O)N[C@H]2[C@@H]3COC[C@@H]32)c(C(OC)c2ccccc2)o1] | 339.2 | 0.77 (formic) |
| 67 | (+/-)-N⁴-(2-Hydroxypropyl)-N²- methyl-5-((S)-1- phenylethyl)furan-2,4- dicarboxamide | 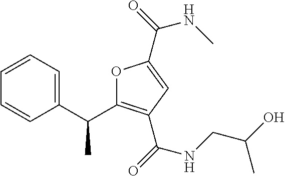 [SMILES: CNC(=O)c1cc(C(=O)NCC(C)O)c([C@@H](C)c2ccccc2)o1] | 331.1 | 0.77 (formic) |
| 68 | (+/-)-5-(1-(3- Fluorophenyl)ethyl)-N⁴-((1r,4r)- 4-hydroxycyclohexyl)-N²- methylfuran-2,4-dicarboxamide | 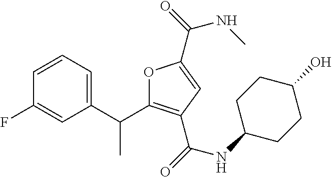 [SMILES: CNC(=O)c1cc(C(=O)N[C@H]2CC[C@H](O)CC2)c(C(C)c2cccc(F)c2)o1] | 389.4 | 0.83 (formic) |
| 69 | (+/-)-N⁴-((1R,5S,6r)-3- Oxabicyclo[3.1.0]hexan-6-yl)-5- (1-(3-chlorophenyl)-2- hydroxyethyl)-N²-methylfuran- 2,4-dicarboxamide | 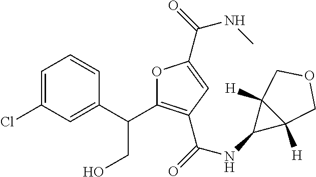 [SMILES: CNC(=O)c1cc(C(=O)N[C@H]2[C@@H]3COC[C@@H]32)c(C(CO)c2cccc(Cl)c2)o1] | 405.3 | 0.78 (formic) |
| 70 | (+/-)-N⁴-Ethyl-N²-methyl-5-(1- phenylethyl)furan-2,4- dicarboxamide | 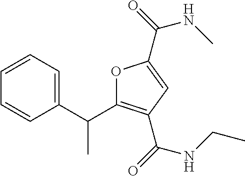 [SMILES: CCNC(=O)c1cc(C(=O)NC)oc1C(C)c1ccccc1] | 301.3 | 0.86 (formic) |
| 71 | (+/-)-N⁴-(sec-Butyl)-N²-methyl- 5-((S)-1-phenylethyl)furan-2,4- dicarboxamide | 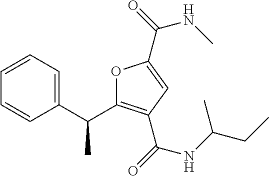 [SMILES: CCC(C)NC(=O)c1cc(C(=O)NC)oc1[C@@H](C)c1ccccc1] | 329.2 | 1.00 (formic) |
| 72 | 5-Benzyl-N⁴-((1R,5S,6r)-3- oxabicyclo[3.1.0]hexan-6-yl)-N²- methylfuran-2,4-dicarboxamide | 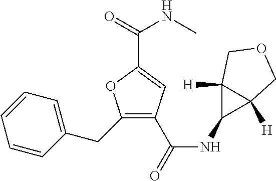 [SMILES: CNC(=O)c1cc(C(=O)N[C@H]2[C@@H]3COC[C@@H]32)c(Cc2ccccc2)o1] | 341.2 | 0.80 (formic) |
| 73 | (S)-N⁴-(2-Hydroxyethyl)-N²- methyl-5-(1-phenylethyl)furan- 2,4-dicarboxamide | 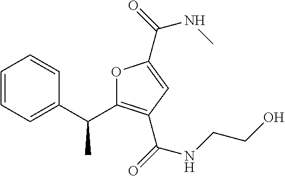 [SMILES: CNC(=O)c1cc(C(=O)NCCO)c([C@@H](C)c2ccccc2)o1] | 317.2 | 0.75 (formic) |
| 74 | N⁴-(1-Hydroxypropan-2-yl)-N²- methyl-5-((S)-1- phenylethyl)furan-2,4- dicarboxamide | 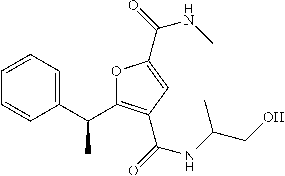 [SMILES: CNC(=O)c1cc(C(=O)NC(C)CO)c([C@@H](C)c2ccccc2)o1] | 331.2 | 0.77 (formic) |
| 75 | (+/-)-5-(1-(3,4- Dimethoxyphenyl)ethyl)-N⁴- ((1r,4r)-4-hydroxycyclohexyl)- N²-methylfuran-2,4- dicarboxamide | 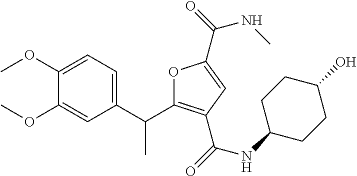 [SMILES: CNC(=O)c1cc(C(=O)N[C@H]2CC[C@H](O)CC2)c(C(C)c2ccc(OC)c(OC)c2)o1] | 431.4 | 0.73 (formic) |
| 76 | (S)-N⁴-(3-Hydroxypropyl)-N²- methyl-5-(1-phenylethyl)furan- 2,4-dicarboxamide | 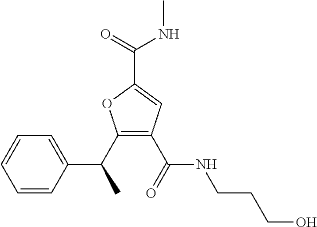 [SMILES: CNC(=O)c1cc(C(=O)NCCCO)c([C@@H](C)c2ccccc2)o1] | 331.2 | 0.77 (formic) |
| 77 | (+/-)-N⁴-((1r,4r)-4- Hydroxycyclohexyl)-5- (methoxy(phenyl)methyl)-N²- methylfuran-2,4-dicarboxamide | 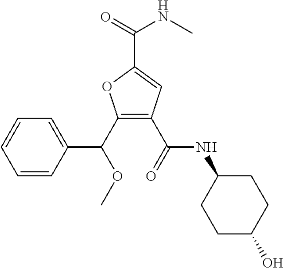 [SMILES: CNC(=O)c1cc(C(=O)N[C@H]2CC[C@H](O)CC2)c(C(OC)c2ccccc2)o1] | 409.4 [MNa]⁺ | 0.74 (formic) |
| 78 | Mixture of (+/-)-N⁴-((1S,3R,4R)- 4-Hydroxy-3-methylcyclohexyl)- N²-methyl-5-((S)-1- phenylethyl)furan-2,4- dicarboxamide and (+/-)-N⁴- ((1S,3R,4R)-4-hydroxy-3- methylcyclohexyl)-N²-methyl-5- ((R)-1-phenylethyl)furan-2,4- dicarboxamide | 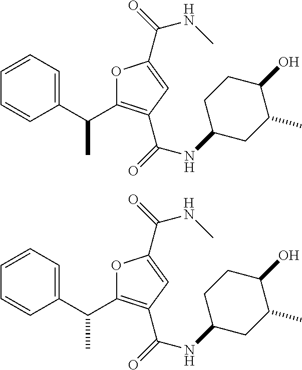 | 385.4 | 0.89 (formic) |
| 79 | (+/-)-N⁴-((1R,5S,6r)-3- Oxabicyclo[3.1.0]hexan-6-yl)-5- (hydroxy(phenyl)methyl)-N²- methylfuran-2,4-dicarboxamide | 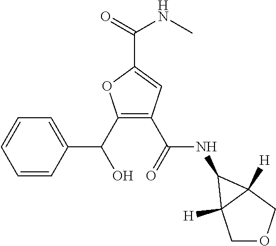 [SMILES: CNC(=O)c1cc(C(=O)N[C@H]2[C@@H]3COC[C@@H]32)c(C(O)c2ccccc2)o1] | 379.3 [MNa]⁺ | 0.70 (formic) |
| 80 | 5-((R*)-2-Cyano-1-phenylethyl)- N⁴-((1r,4R)-4- hydroxycyclohexyl)-N²- methylfuran-2,4-dicarboxamide | 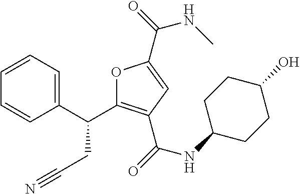 [SMILES: CNC(=O)c1cc(C(=O)N[C@H]2CC[C@H](O)CC2)c([C@H](CC#N)c2ccccc2)o1] | 396.3 | 0.74 (formic) |
| 81 | (S)-N⁴-(2-Methoxyethyl)-N²- methyl-5-(1-phenylethyl)furan- 2,4-dicarboxamide | 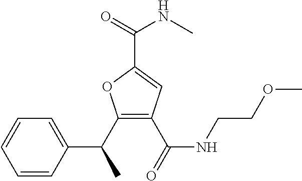 [SMILES: CNC(=O)c1cc(C(=O)NCCOC)c([C@@H](C)c2ccccc2)o1] | 331.2 | 0.85 (formic) |
| 82 | 5-((R*)-1-(3,5- Dichlorophenyl)ethyl)-N4- ((1r,4R)-4-hydroxycyclohexyl)- N²-methylfuran-2,4- dicarboxamide | 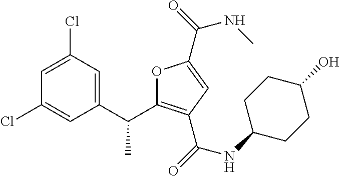 [SMILES: CNC(=O)c1cc(C(=O)N[C@H]2CC[C@H](O)CC2)c([C@H](C)c2cc(Cl)cc(Cl)c2)o1] | 439.3 | 1.00 (formic) |
| 83 | N⁴-((1r,4R)-4- Hydroxycyclohexyl)-5-((R*)-1- (4-methoxy-3- methylphenyl)ethyl)-N²- methylfuran-2,4-dicarboxamide | 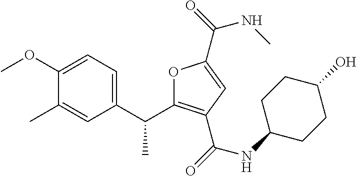 [SMILES: CNC(=O)c1cc(C(=O)N[C@H]2CC[C@H](O)CC2)c([C@H](C)c2ccc(OC)c(C)c2)o1] | 415.4 | 0.86 (formic) |
| 83 | N⁴-((1r,4R)-4- Hydroxycyclohexyl)-N²-methyl- 5-((R*)-1-phenylethyl)furan-2,4- dicarboxamide | 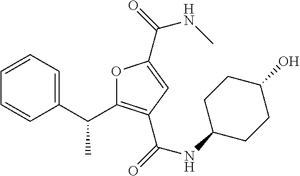 [SMILES: CNC(=O)c1cc(C(=O)N[C@H]2CC[C@H](O)CC2)c([C@H](C)c2ccccc2)o1] | 371.4 | 0.79 (formic) |
| 85 | 5-((R*)-1-(3- Chlorophenyl)ethyl)-N⁴-((1r,4R)- 4-hydroxycyclohexyl)-N²- methylfuran-2,4-dicarboxamide | 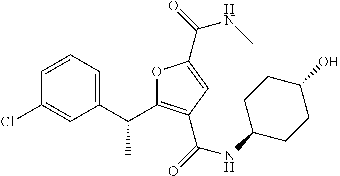 [SMILES: CNC(=O)c1cc(C(=O)N[C@H]2CC[C@H](O)CC2)c([C@H](C)c2cccc(Cl)c2)o1] | 405.4 | 0.89 (formic) |
| 86 | 5-((R*)-1-(3- Fluorophenyl)ethyl)-N⁴-((1r,4R)- 4-hydroxycyclohexyl)-N²- methylfuran-2,4-dicarboxamide | 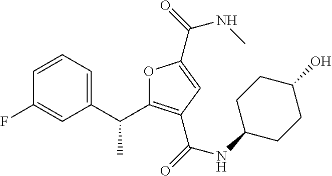 [SMILES: CNC(=O)c1cc(C(=O)N[C@H]2CC[C@H](O)CC2)c([C@H](C)c2cccc(F)c2)o1] | 389.4 | 0.83 (formic) |
| 87 | N⁴-((1R,5S,6r)-3- Oxabicyclo[3.1.0]hexan-6-yl)- N²-methyl-5-((R)-1- phenylethyl)furan-2,4- dicarboxamide | 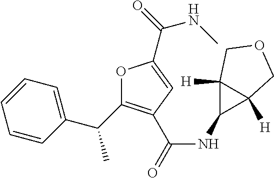 [SMILES: CNC(=O)c1cc(C(=O)N[C@H]2[C@@H]3COC[C@@H]32)c([C@H](C)c2ccccc2)o1] | 355.5 | 0.82 (formic) |
| 88 | N⁴-((1R,5S,6r)-3- Oxabicyclo[3.1.0]hexan-6-yl)-5- ((R*)-1-(3-chlorophenyl)-2- cyanoethyl)-N²-methylfuran-2,4- dicarboxamide | 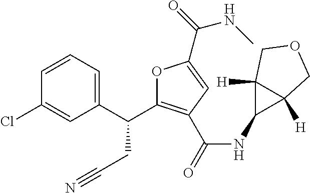 [SMILES: CNC(=O)c1cc(C(=O)N[C@H]2[C@@H]3COC[C@@H]32)c([C@H](CC#N)c2cccc(Cl)c2)o1] | 414.3 | 0.86 (formic) |
| 89 | N⁴-((1R,5S,6r)-3- Oxabicyclo[3.1.0]hexan-6-yl)-5- ((R*)-1-(3-chlorophenyl)ethyl)- N²-methylfuran-2,4- dicarboxamide | 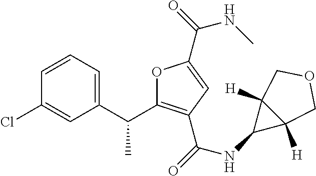 [SMILES: CNC(=O)c1cc(C(=O)N[C@H]2[C@@H]3COC[C@@H]32)c([C@H](C)c2cccc(Cl)c2)o1] | 389.3 | 0.93 (formic) |
| 90 | 5-((S*)-1-(4- Chlorophenyl)ethyl)-N⁴-((1r,4S)- 4-hydroxycyclohexyl)-N²- methylfuran-2,4-dicarboxamide | 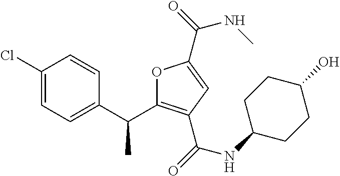 [SMILES: CNC(=O)c1cc(C(=O)N[C@H]2CC[C@H](O)CC2)c([C@@H](C)c2ccc(Cl)cc2)o1] | 405.1 | 0.92 (high pH) |
| 91 | (R*)-N⁴-Ethyl-N²-methyl-5-(1- phenylethyl)furan-2,4- dicarboxamide | 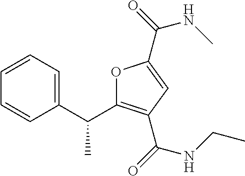 [SMILES: CCNC(=O)c1cc(C(=O)NC)oc1[C@H](C)c1ccccc1] | 301.3 | 0.86 (formic) |
| 92 | N⁴-((1R,3S,5S,6r)-3- Hydroxybicyclo[3.1.0]hexan-6- yl)-N²-methyl-5-((S*)-1- phenylethyl)furan-2,4- dicarboxamide | 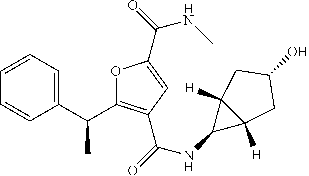 [SMILES: CNC(=O)c1cc(C(=O)N[C@H]2[C@@H]3C[C@H](O)C[C@@H]32)c([C@@H](C)c2ccccc2)o1] | 369.5 | 0.82 (formic) |
| 93 | 5-((S*)-(3- Chlorophenyl)(methoxy)methyl)- N⁴-((1r,4S)-4- hydroxycyclohexyl)-N²- methylfuran-2,4-dicarboxamide | 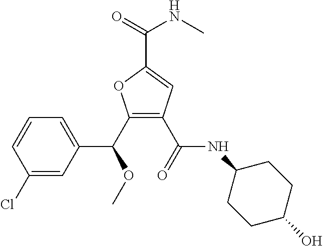 [SMILES: CNC(=O)c1cc(C(=O)N[C@H]2CC[C@H](O)CC2)c([C@@H](OC)c2cccc(Cl)c2)o1] | 389.3, 391.3 | 0.85 (formic) |
| 94 | 5-((S*)-1-(3-Chloro-5- methoxyphenyl)ethyl)-N⁴- ((1r,4S)-4-hydroxycyclohexyl)- N²-methylfuran-2,4- dicarboxamide | 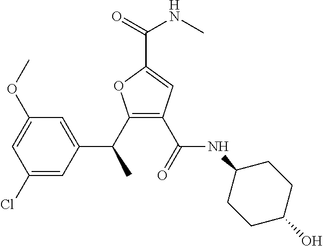 [SMILES: CNC(=O)c1cc(C(=O)N[C@H]2CC[C@H](O)CC2)c([C@@H](C)c2cc(Cl)cc(OC)c2)o1] | 435.3, 437.3 | 0.91 (formic) |
| 95 | 5-((S*)-1-(3-Chloro-4- methoxyphenyl)ethyl)-N⁴- ((1r,4S)-4-hydroxycyclohexyl)- N²-methylfuran-2,4- dicarboxamide | 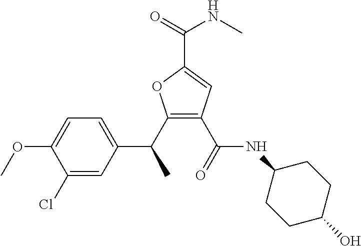 [SMILES: CNC(=O)c1cc(C(=O)N[C@H]2CC[C@H](O)CC2)c([C@@H](C)c2ccc(OC)c(Cl)c2)o1] | 435.3, 437.3 | 0.86 (formic) |
| 96 | N²-methyl-N⁴-((1S,2S)-2- Methylcyclopropyl)-5-((S*)-1- phenylethyl)furan-2,4- dicarboxamide | 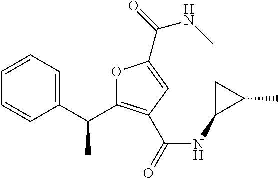 [SMILES: CNC(=O)c1cc(C(=O)N[C@H]2C[C@@H]2C)c([C@@H](C)c2ccccc2)o1] | 369.5 | 0.82 (formic) |
| 97 | N⁴-((1R,3R,5S,6r)-3- Hydroxybicyclo[3.1.0]hexan-6- yl)-N²-methyl-5-((S*)-1- phenylethyl)furan-2,4- dicarboxamide | 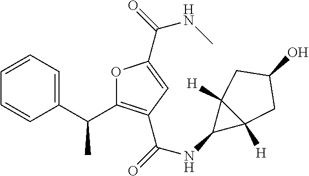 [SMILES: CNC(=O)c1cc(C(=O)N[C@H]2[C@@H]3C[C@@H](O)C[C@@H]32)c([C@@H](C)c2ccccc2)o1] | 369.5 | 0.78 (formic) |
| 98 | 5-((S*)-1-(3-Chloro-4- methylphenyl)ethyl)-N⁴-((1r,4S)- 4-hydroxycyclohexyl)-N²- methylfuran-2,4-dicarboxamide | 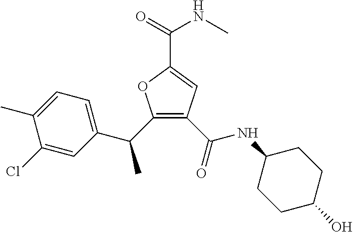 [SMILES: CNC(=O)c1cc(C(=O)N[C@H]2CC[C@H](O)CC2)c([C@@H](C)c2ccc(C)c(Cl)c2)o1] | 419.4, 421.3 | 0.95 (formic) |
| 99 | N⁴-((1r,4S)-4- Hydroxycyclohexyl)-N²-methyl- 5-((S*)-1-(p-tolyl)ethyl)furan- 2,4-dicarboxamide | 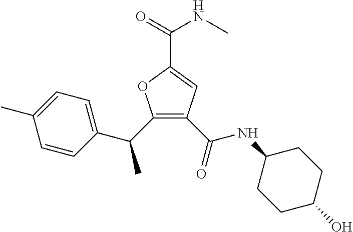 [SMILES: CNC(=O)c1cc(C(=O)N[C@H]2CC[C@H](O)CC2)c([C@@H](C)c2ccc(C)cc2)o1] | 369.5 | 0.78 (formic) |
| 100 | N⁴-((1r,4S)-4- Methoxycyclohexyl)-N²-methyl- 5-((S*)-1-phenylethyl)furan-2,4- dicarboxamide | 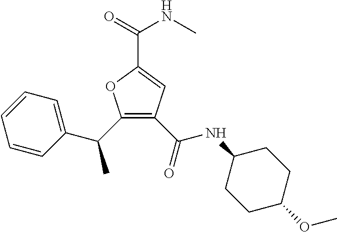 [SMILES: CNC(=O)c1cc(C(=O)N[C@H]2CC[C@H](OC)CC2)c([C@@H](C)c2ccccc2)o1] | 385.6 | 0.94 (formic) |
| 101 | 5-((R*)-2-Hydroxy-1- phenylethyl)-N⁴-((1r,4R)-4- hydroxycyclohexyl)-N²- methylfuran-2,4-dicarboxamide | 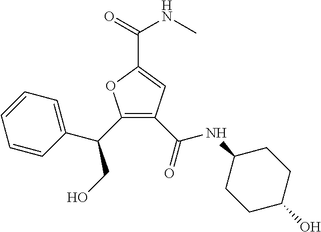 [SMILES: CNC(=O)c1cc(C(=O)N[C@H]2CC[C@H](O)CC2)c([C@@H](CO)c2ccccc2)o1] | 387.4 | 0.64 (formic) |
| 102 | N⁴-((1r,4S)-4- Hydroxycyclohexyl)-5-((S*)-1- (4-methoxyphenyl)ethyl)-N²- methylfuran-2,4-dicarboxamide | 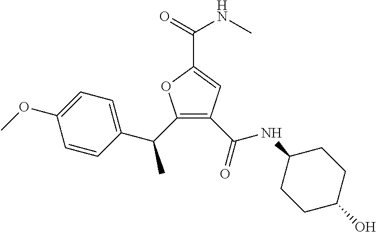 [SMILES: CNC(=O)c1cc(C(=O)N[C@H]2CC[C@H](O)CC2)c([C@@H](C)c2ccc(OC)cc2)o1] | 401.4 | 0.79 (formic) |
| 103 | 5-((S*)-1-(2- Chlorophenyl)ethyl)-N⁴-((1r,4S)- 4-hydroxycyclohexyl)-N²- methylfuran-2,4-dicarboxamide | 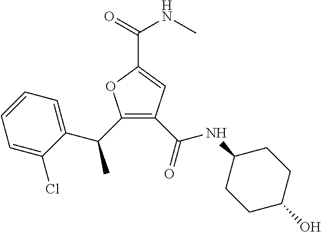 [SMILES: CNC(=O)c1cc(C(=O)N[C@H]2CC[C@H](O)CC2)c([C@@H](C)c2ccccc2Cl)o1] | 405.4, 407.3 | 0.84 (formic) |
| 104 | (S)-N⁴-(Cyclopropylmethyl)-N²- methyl-5-(1-phenylethyl)furan- 2,4-dicarboxamide | 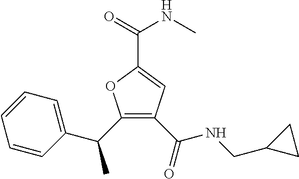 [SMILES: CNC(=O)c1cc(C(=O)NCC2CC2)c([C@@H](C)c2ccccc2)o1] | 327.2 | 0.89 (formic) |
| 105 | N⁴-((1r,4S)-4- Hydroxycyclohexyl)-N²-methyl- 5-((S*)-1-(m-tolyl)ethyl)furan- 2,4-dicarboxamide | 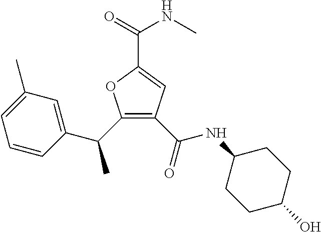 [SMILES: CNC(=O)c1cc(C(=O)N[C@H]2CC[C@H](O)CC2)c([C@@H](C)c2cccc(C)c2)o1] | 385.4 | 0.86 (formic) |
| 106 | 5-((S*)-1-(3,4- Dimethoxyphenyl)ethyl)-N⁴- ((1r,4S)-4-hydroxycyclohexyl)- N²-methylfuran-2,4- dicarboxamide | 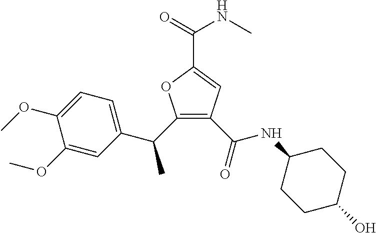 [SMILES: CNC(=O)c1cc(C(=O)N[C@H]2CC[C@H](O)CC2)c([C@@H](C)c2ccc(OC)c(OC)c2)o1] | 431.3 | 0.72 (formic) |
| 107 | N⁴-(1r,4S)-4- Hydroxycyclohexyl)-5-((S*)-1- (3-methoxyphenyl)ethyl)-N²- methylfuran-2,4-dicarboxamide | 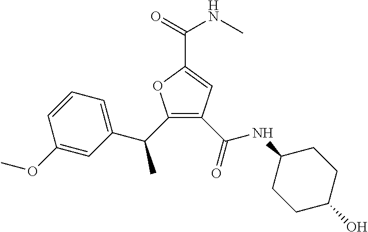 [SMILES: CNC(=O)c1cc(C(=O)N[C@H]2CC[C@H](O)CC2)c([C@@H](C)c2cccc(OC)c2)o1] | 401.4 | 0.80 (formic) |
| 108 | N⁴-((1r,4S)-4- Hydroxycyclohexyl)-5-((S*)- methoxy(phenyl)methyl)-N²- methylfuran-2,4-dicarboxamide | 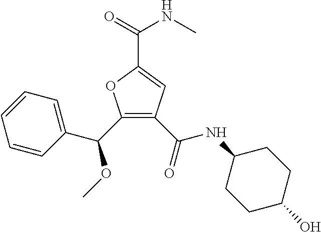 [SMILES: CNC(=O)c1cc(C(=O)N[C@H]2CC[C@H](O)CC2)c([C@@H](OC)c2ccccc2)o1] | 355.3 for [M − OMe]⁺ | 0.75 (formic) |
| 109 | (S*)-N⁴-Cyclopropyl-N²-methyl- 5-(1-phenylethyl)furan-2,4- dicarboxamide | 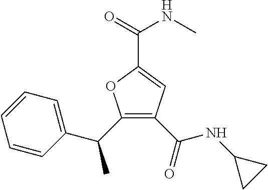 [SMILES: CNC(=O)c1cc(C(=O)NC2CC2)c([C@@H](C)c2ccccc2)o1] | 313.2 | 0.87 (formic) |
| 110 | N⁴-(1R,3r,5S,6r)-3- Hydroxybicyclo[3.1.0]hexan-6- yl)-N²-methyl-5-(1- phenylethyl)furan-2,4- dicarboxamide | 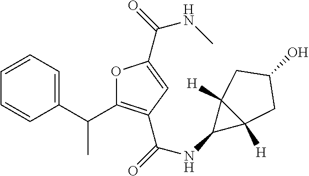 [SMILES: CNC(=O)c1cc(C(=O)N[C@H]2[C@@H]3C[C@H](O)C[C@@H]32)c(C(C)c2ccccc2)o1] | 369.3 | 0.77 (formic) |

### Example 111:5-(1-(1H-pyrrolo[2,3-b]pyridin-4-yl)ethyl)-N⁴-cyclopropyl-N²-methylfuran-2,4-dicarboxamide

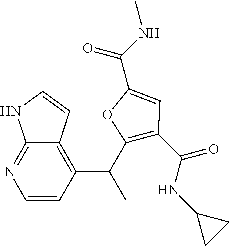 [SMILES: CNC(=O)c1cc(C(=O)NC2CC2)c(C(C)c2ccnc3[nH]ccc23)o1]

2-(1-(1H-pyrrolo[2,3-b]pyridin-4-yl)ethyl)-5-(methylcarbamoyl)furan-3-carboxylic acid (for a preparation see Intermediate 38, 1.08 g, 1.90 mmol), HATU (1.08 g, 2.84 mmol) were dissolved in DCM (15 mL), DIPEA (0.662 mL, 3.79 mmol) was added and the mixture was stirred for 15 minutes. Then, cyclopropanamine (0.197 mL, 2.84 mmol) was added and this was stirred for 2 hours. The reaction was partitioned between DCM and saturated sodium bicarbonate solution. The aqueous layer was washed with DCM twice. The organic layers were combined, dried over Na₂SO₄, filtrated and concentrated in vacuo. The crude product was applied to a 50 g ULTRA silica SNAP cartridge in the minimum of DCM+Methanol and eluted with 0% (3:1 EtOAc:EtOH) in cyclohexane for 2 column volumes then 0-100% (3:1 EtOAc:EtOH) over 14 column volumes then held at 100% for 2 column volumes. The appropriate fractions were combined and evaporated under reduced pressure to give the desired compound as a pale yellow solid (478 mg, 1.33 mmol, 70.1% yield).

LCMS (2 min Formic): Rt=0.52 min, [MH]⁺=353.3

### Biological Data

The compounds of formula (I) may be tested in one or more of the following assays:

### Time Resolved Fluorescence Resonance Energy Transfer (TR-FRET) Assay

Bromodomain binding was assessed utilising a time resolved fluorescent resonance energy transfer (TR-FRET) competition assay. To enable this approach a known, high affinity, pan-BET interacting small molecule was labelled with Alexa Fluor® 647, which is a far-red-fluorescent dye (Reference Compound X). Reference Compound X acts as a reporter of bromodomain binding and is the acceptor fluorophore component of the TR-FRET pair. Europium chelate, conjugated to an anti-6*His antibody, was utilised as the donor fluorophore in the TR-FRET pair. The anti-6*His antibody binds selectively to a six Histidine purification epitope added to the amino-terminus of each of the BET tandem bromodomain protein constructs used in this study. A TR-FRET signal is generated when the donor and acceptor fluorophores are in close proximity, between 20-80 Å, which is enabled in this assay by binding of Reference Compound X to the bromodomain protein.

### Reference Compound X: 4-((Z)-3-(6-((5-(2-((45)-6-(4-chlorophenyl)-8-methoxy-1-methyl-4H-benzo[f][1,2,4]triazolo[4,3-a][1,4]diazepin-4-yl)acetamido)pentyl)amino)-6-oxohexyl)-2-((2E,4E)-5-(3,3-dimethyl-5-sulfo-1-(4-sulfobutyl)-3H-indol-1-ium-2-yl)penta-2,4-dien-1-ylidene)-3-methyl-5-sulfoindolin-1-yl)butane-1-sulphonate)

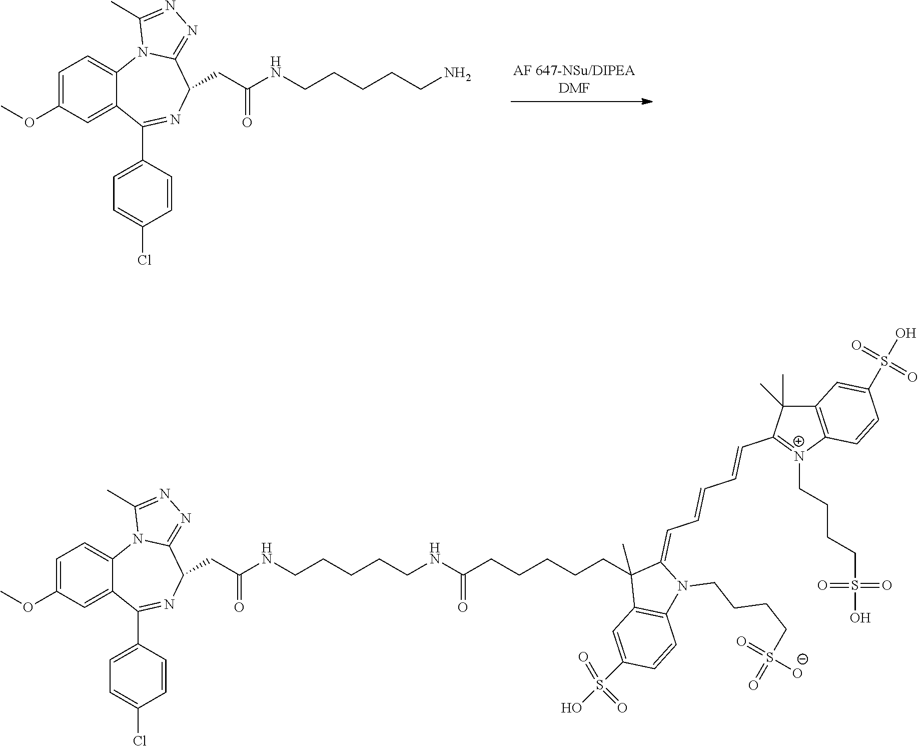 [SMILES: COc1ccc2c(c1)C(c1ccc(Cl)cc1)=N[C@@H](CC(=O)NCCCCCN)c1nnc(C)n1-2 ; COc1ccc2c(c1)C(c1ccc(Cl)cc1)=N[C@@H](CC(=O)NCCCCCNC(=O)CCCCCC1(C)/C(=C/C=C/C=C/C3=[N+](CCCCS(=O)(=O)O)c4ccc(S(=O)(=O)O)cc4C3(C)C)N(CCCCS(=O)(=O)[O-])c3ccc(S(=O)(=O)O)cc31)c1nnc(C)n1-2]

To a solution of N-(5-aminopentyl)-2-((45)-6-(4-chlorophenyl)-8-methoxy-1-methyl-4H-benzo[f][1,2,4]triazolo[4,3-a][1,4]diazepin-4-yl)acetamide (for a preparation see Reference Compound J, WO2011/054848A1, 1.7 mg, 3.53 μmol) in DMF (40 μL) was added a solution of AlexaFluor647-ONSu (2.16 mg, 1.966 μmol) also in DMF (100 μL). The mixture was basified with DIPEA (1 μl, 5.73 μmol) and agitated overnight on a vortex mixer.

The reaction mixture was evaporated to dryness. The solid was dissolved in MeCN/water/AcOH (5/4/1, <1 mL) filtered and was applied to a Phenomenex Jupiter C18 preparative column and eluted with the following gradient (A=0.1% trifluoroacetic acid in water, B=0.1% TFA/90% MeCN/10% water): Flow rate=10 mL/min., AU=20/10 (214 nm):

5-35%, t=0 min: B=5%; t=10 min: B=5%; t=100 min: B=35%; t=115 min: B=100% (Sep. grad: 0.33%/min)

The major component was eluted over the range 26-28% B but appeared to be composed of two peaks. The middle fraction (F1.26) which should contain “both” components was analysed by analytical HPLC (Spherisorb ODS2, 1 to 35% over 60 min): single component eluting at 28% B.

Fractions F1.25/26&27 were combined and evaporated to dryness. Transferred with DMF, evaporated to dryness, triturated with dry ether and the blue solid dried overnight at <0.2 mbar: 1.54 mg.

Analytical HPLC (Sphersisorb ODS2, 1 to 35% B over 60 min): MSM10520-1: [M+H]⁺ (obs): 661.8/− corresponding with M-29. This equates to [(M+2H)/2]⁺ for a calculated mass of 1320.984 which is M-29. This is a standard occurrence with the Alexa Fluor 647 dye and represents a theoretical loss of two methylene groups under the conditions of the mass spectrometer.

Assay Principle: In order to generate a TR-FRET signal, donor fluorophore is excited by a laser at λ337 nm, which subsequently leads to emission at λ618 nm. If the acceptor fluorophore is in close proximity then energy transfer can occur, which leads to emission of Alexa Fluor® 647 at λ665 nm. In the presence of competitor compound, Reference Compound X can be displaced from binding to the bromodomain. If displacement occurs, the acceptor fluorophore is no longer in proximity to the donor fluorophore, which prevents fluorescent energy transfer and, subsequently, a loss of Alexa Fluor®647 emission at λ665 nm.

The competition of the compounds of formula (I) with Reference Compound X for binding to the BET family (BRD2, BRD3, BRD4 and BRDT) was assessed using protein truncates spanning both bromodomain 1 (BD1) and bromodomain 2 (BD2). In order to monitor differential binding to either BD1 or BD2, single residue mutations of key tyrosines to alanine were made in the acetyl lysine binding pockets. To validate this approach, a double residue mutant tandem domain protein was produced for each of the BET family members. Utilising a Fluorescence Polarisation approach, binding affinities for each of the single and double mutants for Reference Compound X were determined. The affinities of the double mutant tandem proteins for Reference Compound X were greatly greatly reduced in comparison to the non mutated, wild type tandem BET proteins (>1000 fold reduction in Kd). The affinities of the single mutated bromdomain tandem proteins for Reference Compound X were equi-potent with the corresponding non-mutated BET protein. These data demonstrated that single mutations of Tyrosine to Alanine reduce the Kd of the interaction between the mutated bromodomain and Reference Compound X by >1000 fold. In the TR-FRET competition assay, Reference Compound X is used at a concentration that is equivalent to the Kd for the non-mutated bromodomain, which ensures that no binding at the mutated bromodomain is detected.

Protein production: Recombinant Human Bromodomains [(BRD2 (1-473) (Y113A) and (Y386A), BRD3 (1-435) (Y73A) and (Y348A) BRD4 (1-477) (Y97A) and (Y390A) and BRDT (1-397) (Y66A) and (Y309A)] were expressed in E. coli cells (in pET15b vector for BRD2/3/4 and in pET28a vector for BRDT) with a 6-His tag at the N-terminal. The His-tagged Bromodomain pellet was resuspended in 50 mM HEPES (pH7.5), 300 mM NaCl, 10 mM imidazole & 1 μL/mL protease inhibitor cocktail and extracted from the E. coli cells using sonication and purified using a nickel sepharose high performance column, the proteins were washed and then eluted with a linear gradient of 0-500 mM imidazole with buffer 50 mM HEPES (pH7.5), 150 mM NaCl, 500 mM imidazole, over 20 column volumes. Final purification was completed by Superdex 200 prep grade size exclusion column. Purified protein was stored at −80° C. in 20 mM HEPES pH 7.5 and 100 mM NaCl. Protein identity was confirmed by peptide mass fingerprinting and predicted molecular weight confirmed by mass spectrometry.

Protocol for Bromodomain BRD2, 3, 4 and T, BD1+BD2 mutant TR-FRET competition assays: All assay components were dissolved in an assay buffer composing of 50 mM HEPES pH7.4, 50 mM NaCl, 5% Glycerol, 1 mM DTT and 1 mM CHAPS. Reference Compound X was diluted, in assay buffer containing 20 nM single mutant, tandem bromodomain protein, to a concentration equivalent to 2*Kd for this bromodomain. The solution containing bromodomain and Reference Compound X was added to dose response dilutions of test compound or DMSO vehicle (a maximum of 0.5% DMSO is used in this assay) in Greiner 384 well black low volume microtitre plates and subsequently incubated for 30 minutes at rt. An equal volume of 3 nM of anti-6*His Europium chelate was added to all wells, followed by a further 30 minute incubation at rt. TR-FRET was detected using a Perkin Elmer Multimode plate reader, by exciting the donor fluorophore at λ337 nm and subsequently, after a delay of 50 μsecs, measuring emission of the donor and acceptor fluorophores at λ615 nm and λ665 nm, respectively. In order to control these assays, 16 replicates each of uninhibited (DMSO vehicle) and inhibited (10*IC₅₀ concentrations of Example 11 of WO 2011/054846A1) TR-FRET assays were included on every microtitre plate.

cA four parameter curve fit of the following form was then applied:

y=a+((b−a)/(1+(10\hat{ }x/10\hat{ }c)\hat{ }d)
Where ‘a’ is the minimum, ‘b’ is the Hill slope, ‘c’ is the pIC₅₀ and ‘d’ is the maximum.

With the exception of Examples 15 and 17, all compounds (Examples) were each tested in the BRD4 BD1 and the BRD4 BD2 TR-FRET assays essentially as described above. Those of skill in the art will recognise that in vitro binding assays and cell-based assays for functional activity are subject to experimental variability. Accordingly, it is to be understood that the pIC₅₀ values given below are exemplary only. pIC₅₀ values are expressed as log₁₀ units.

All tested Examples, with the exception of Example 109, were found to have a pIC₅₀ 5.0 in at least one assay described above.

Examples 87-108 were found to have a pIC₅₀ 5.0 and <6.0 in the BRD4 BD2 assay.

All other compounds were found to have a pIC₅₀ 6.0 and <8.2 in the BRD4 BD2 assay. In particular, Example 1 was found to have a pIC₅₀ of 7.9 in the BRD4 BD2 assay; Example 3 was found to have a pIC₅₀ of 7.9 in the BRD4 BD2 assay; Example 8 was found to have a pIC₅₀ of 7.7 in the BRD4 BD2 assay; and Example 13 was found to have a pIC₅₀ of 7.4 in the BRD4 BD2 assay.

Calculation of Selectivity for BRD4 BD2 Over BRD4 BD1

Selectivity for BRD4 BD2 over BRD4 BD1 was calculated as follows:

Selectivity=BRD4 BD2 pIC₅₀−BRD4 BD1 pIC₅₀

All tested Examples, with the exemption of Examples 91 and 104-109 were found to have selectivity for BRD4 BD2 over BRD4 BD1 of ≥1 log unit in at least one of the TR-FRET assays described above, and hence are at least 10 fold selective for BRD4 BD2 over BRD4 BD1.

Examples 1-14, 18-80, 82, 110 and 111 were found to have selectivity for BRD4 BD2 over BRD4 BD1 of ≥2 log unit in at least one of the TR-FRET assays described above, and hence are at least 100 fold selective for BRD4 BD2 over BRD4 BD1.

Examples 1, 3, 5, 7, 8, 9, 10, 12, 18-31, 34, 35, 36, 40, 41 and 110 were found to have selectivity for BRD4 BD2 over BRD4 BD1 of ≥3 log unit in at least one of the TR-FRET assays described above, and hence are at least 1000 fold selective for BRD4 BD2 over BRD4 BD1.

Example 1 was found to have a selectivity for BRD4 BD2 over BRD4 BD1 of 3.2 log units in at least one of the TR-FRET assays described above.

Example 3 was found to have a selectivity for BRD4 BD2 over BRD4 BD1 of 3.2 log units in at least one of the TR-FRET assays described above.

Example 8 was found to have a selectivity for BRD4 BD2 over BRD4 BD1 of 3.1 log units in at least one of the TR-FRET assays described above.

Example 13 was found to have a selectivity for BRD4 BD2 over BRD4 BD1 of 2.9 log units in at least one of the TR-FRET assays described above.

1. A compound of formula (I) 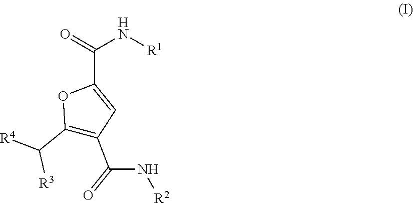
or a pharmaceutically acceptable salt thereof
wherein:
R¹ is —C₁₋₃alkyl or cyclopropyl;
R² is —C₀₋₃alkyl-cycloalkyl, wherein the cycloalkyl group is optionally substituted with one, two or three R⁵ groups which may be the same or different; or
R² is —C₀₋₄alkyl-heterocyclyl or —(CH₂)ₚO-heterocyclyl wherein each heterocyclyl is optionally substituted by one or two R⁹ groups which may be the same or different; or
R² is H, —CH₃, —C₂₋₆alkyl optionally substituted by one, two, three, four or five fluoro, —C₂₋₆alkylOR⁶, —C₂₋₆alkylNR^{10a}R^{11a}, —(CH₂)ₘSO₂C₁₋₃alkyl, —(CH₂)ₘSO₂NR¹⁰R¹¹, —(CH₂)ₘC(O)NR¹⁰R¹¹, —(CH₂)ₘCN, —(CH₂)ₘCO₂R⁶, —(CH₂)ₘNHCO₂C₁₋₄alkyl, —(CH₂)ₘNHC(O)C₁₋₄alkyl or —(CH₂)ₙheteroaryl wherein the heteroaryl is optionally substituted by one or two R⁷ groups which may be the same or different;
R³ is H, —C₁₋₄alkyl, cyclopropyl, fluoro, chloro, —CH₂F, —C₀₋₃alkylOR⁵ or —C₀₋₃alkylCN;
R⁴ is phenyl or a heteroaryl group wherein each are optionally substituted by one, two or three R⁷ groups which may be the same or different;
each R⁵ is independently halo, —C₀₋₆alkyl-R⁸, —O—C₂₋₆alkyl-R⁸, —OCH₂phenyl, —CN, or —SO₂C₁₋₃alkyl;
R⁶ is H or —C₁₋₄alkyl;
each R⁷ is independently oxo, halo, —C₁₋₄alkyl optionally substituted by one, two or three fluoro, —C₀₋₃alkylOR⁶, —OC₂₋₃alkylOR⁶, —C₀₋₃alkylNR¹⁰R¹¹, —C₀₋₃alkyl-CONR¹⁰R¹¹, —CN, —SO₂—C₁₋₃alkyl, —SO₂NR¹⁰R¹¹ or —SO₂phenyl optionally substituted by —C₁₋₄alkyl;
R⁸ is H, —OR⁶, —NR¹⁰R¹¹ or heteroaryl;
each R⁹ is independently halo, —C₁₋₄alkyl, cyclopropyl, cyclobutyl, —CH₂CF₃, —CH₂CHF₂, —CH₂CH₂F, —OCH₂CH₂OR⁶, —C₀₋₃alkylOR⁶, —C₀₋₃alkylNR¹⁰R¹¹, —NHCH₂CH₂OR⁶, —NHCO₂C₁₋₄alkyl, oxo, —C(O)R⁶, —C(O)OR⁶ or —C(O)NR¹⁰R¹¹;
R¹⁰ and R¹¹ are each independently selected from H and —C₁₋₃alkyl; or R¹⁰ and R¹¹ may join together with the nitrogen to which they are attached, to form a 4 to 7-membered heterocyclyl optionally substituted by one or two substituents independently selected from —C₁₋₃alkyl optionally substituted with one, two or three fluorine atoms, —C₂₋₄alkylOH, —OH and F;
R^{10a} and R^{11a} are each independently selected from H and —C₁₋₃alkyl;
m is an integer selected from 2, 3 or 4;
n is an integer selected from 0, 1, 2, 3 or 4; and
p is an integer selected from 2, 3 or 4.

2. The compound or pharmaceutically acceptable salt thereof according to claim 1 wherein R¹ is methyl.

3. The compound or pharmaceutically acceptable salt thereof according to claim 1 wherein R² is a —C₀₋₃alkyl-C₃₋₇cycloalkyl group, wherein the C₃₋₇ cycloalkyl group is selected from cyclopropyl, cyclobutyl, cyclohexyl or bicyclo[3.1.0]hexanyl said groups being optionally substituted with one, two or three R⁵ groups which may be the same or different.

4. The compound or pharmaceutically acceptable salt thereof according to claim 3 wherein R² is a cyclohexyl group optionally substituted by one methyl group or is a cyclohexyl group optionally substituted with a OH group.

5. The compound or pharmaceutically acceptable salt thereof according to claim 1 wherein R² is —C₀₋₄alkyl-heterocyclyl wherein the heterocyclyl is selected from oxetanyl, tetrahydrofuranyl, tetrahydro-2H-pyranyl, morpholinyl, piperidinyl, piperazinyl, (1R,5S)-3-oxabicyclo[3.1.0]hexanyl and (1R,5S)-3-azabicyclo[3.1.0]hexanyl said groups being optionally substituted by one or two R⁹ groups which may be the same or different.

6. The compound or pharmaceutically acceptable salt thereof according to claim 1 wherein R² is methyl, ethyl, propyl, iso-propyl, butyl, —CH₂CH₂CH(CH₃)₂, —CH₂CH(CH₃)₂, —CH₂CH₂OR⁶, —CH₂CH₂CH₂OR⁶, —CH₂CH(CH₃)OR⁶, —CH₂CH₂CH(CH₃)OR⁶, —CH₂CH₂CH(CH₃)NR^{10a}R^{11a} , —CH₂CH₂CH₂NR^{10a}R^{11a}, —(CH₂)ₘSO₂CH₃, —(CH₂)ₘC(O)NHCH₃, —(CH₂)ₘCN, —(CH₂)ₘCO₂R⁶, —(CH₂)ₘCF₃ and —(CH₂)ₘNHCO₂C(CH₃)₃.

7. The compound or pharmaceutically acceptable salt thereof according to claim 1 wherein R² is —(CH₂)ₙC₅₋₆heteroaryl wherein the C₅₋₆heteroaryl group is selected from furanyl, thienyl, pyrrolyl, triazolyl, thiazolyl, oxazolyl, isoxazolyl, oxadiazolyl, imidazolyl, pyrazolyl, triazolyl, tetrazolyl, isoxazolyl, pyridinyl, pyridazinyl, pyrazinyl and pyrimidinyl said groups being optionally substituted by one or two substituents independently selected from halo, C₁₋₄alkyl, C₃₋₄cycloalkyl and —C₀₋₃alkylOR⁶.

8. The compound or pharmaceutically acceptable salt thereof according to claim 1 wherein R³ is H, methyl, —CH₂OH, —OMe or —CH₂CN.

9. The compound or pharmaceutically acceptable salt thereof according to claim 1 wherein R⁴ is unsubstituted phenyl or is phenyl substituted by one or two R⁷ groups which may be the same or different selected from halo, —C₁₋₄alkyl, —C₀₋₃alkylOR⁶ and —CN.

10. The compound or pharmaceutically acceptable salt thereof according to claim 1 wherein R⁴ is a heteroaryl group selected from the group consisting of pyridyl, indolyl and pyrrolopyridinyl said groups being optionally substituted by one, two or three R⁷ groups which may be the same or different.

11. (canceled)

12. A compound or a pharmaceutically acceptable salt thereof selected from the group consisting of:
N⁴-((1R,3R,5S,6R)-3-hydroxybicyclo[3.1.0]hexan-6-yl)-N²-methyl-5-((S)-1-phenylethyl)furan-2,4-dicarboxamide;
5-((S*)-1-(3-chlorophenyl)ethyl)-N⁴-((1R,4S)-4-hydroxycyclohexyl)-N²-methylfuran-2,4-dicarboxamide;
N⁴-((1R,4S)-4-Hydroxycyclohexyl)-N²-methyl-5-((S)-1-phenylethyl)furan-2,4-dicarboxamide; and
N⁴-((1R,5S,6R)-3-oxabicyclo[3.1.0]hexan-6-yl)-N²-methyl-5-((S)-1-phenylethyl)furan-2,4-dicarboxamide
or a salt thereof.

13. (canceled)

14. A pharmaceutical composition comprising the compound or pharmaceutically acceptable salt thereof as defined in claim 1 and one or more pharmaceutically acceptable excipients.

15. A combination product comprising the compound or pharmaceutically acceptable salt thereof as defined in claim 1 together with one or more other therapeutically active agents.

16.-23. (canceled)

24. A method of treatment of a disease or condition for which a bromodomain inhibitor is indicated in a subject in need thereof which comprises administering a therapeutically effective amount of the compound or pharmaceutically acceptable salt thereof as defined in claim 1.

25. The method of treatment according to claim 24, wherein the disease or condition is an acute or chronic autoimmune and/or inflammatory condition.

26. The method of treatment according to claim 24, wherein the disease or condition involves an inflammatory response to an infection with bacteria, a virus, fungi, a parasite or their toxins.

27. The method of treatment according to claim 24, wherein the disease or condition is a viral infection.

28. The method of treatment according to claim 24, wherein the disease or condition is cancer.

29. The method of treatment according to claim 24, wherein the disease or condition is rheumatoid arthritis.

30. The method of treatment according to claim 24, wherein the subject is a human.
